(19) United States
(12) Patent Application Publication
(10) Pub. No.: US 2009/0023729 A1
(43) Pub. Date: Jan. 22, 2009

(54) Trisubstituted amine compound

(75) Inventors: Yoshinori Nakamura, Osaka-shi (JP); Norimitsu Hayashi, Osaka-shi (JP); Takanori Higashijima, Osaka-shi (JP); Hitoshi Kubota, Osaka-shi (JP); Kozo Oka, Osaka-shi (JP)

Correspondence Address: BIRCH STEWART KOLASCH & BIRCH, PO BOX 747, FALLS CHURCH, VA, 22040-0747, US

(73) Assignee: Mitsubishi Tanabe Pharma Corporation, Osaka-shi (JP)

(21) Appl. No.: 12/219,776

(22) Filed: Jul. 28, 2008

Related U.S. Application Data
(63) Continuation-in-part of application No. PCT/JP2007/051868, filed on Jan. 30, 2007.
(60) Provisional application No. 60/952,711, filed on Jul. 30, 2007.

(30) Foreign Application Priority Data
Jan. 31, 2006 (JP) 2006-023571
Nov. 30, 2006 (JP) 2006-322854

(51) Int. Cl. A61K 31/506; C07D 401/12; C07D 401/14; C07D 403/12; C07D 413/14; A61K 31/4155; A61P 9/10; A61K 31/4439; C07D 239/47; C07D 239/48; A61K 31/5377

(52) U.S. Cl. 514/235.2; 544/298; 544/331; 546/159; 548/251; 544/122; 544/296; 544/317; 544/323; 514/235.8; 514/272; 514/275; 514/313; 514/381; 514/274

(57) ABSTRACT

The present invention relates to a compound of the general formula (1):

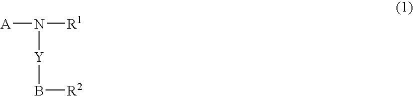

wherein, Y is a methylene group, and the like; A is an optionally substituted heterocyclic group, and the like; B is an optionally substituted heterocyclic group, and the like; R¹ is an optionally substituted alkyl group, wherein the alkyl group further may optionally be substituted by an optionally substituted homocyclic group, and the like; and R² is an optionally substituted amino group, and the like; or a pharmaceutically acceptable derivative thereof, which has an inhibitory activity against cholesteryl ester transfer protein (CETP), thereby being useful for prophylaxis and/or treatment of arteriosclerotic diseases, hyperlipemia or dyslipidemia, and the like.

### TECHNICAL FIELD

The present invention relates to a compound having an inhibitory activity against cholesteryl ester transfer protein (CETP) and showing an activity of increasing HDL cholesterol level and an activity of decreasing LDL cholesterol level, thereby being useful for prophylaxis and/or treatment of arteriosclerotic diseases, hyperlipemia or dyslipidemia.

### BACKGROUND ART

Hypercholesterolemia, especially high serum level of low-density lipoprotein (LDL) cholesterol, has been revealed to be a risk factor of arteriosclerotic diseases by a number of epidemiological surveys. Actually, drugs capable of decreasing LDL cholesterol level such as 3-hydroxy-3-methylglutaryl-coenzyme A (HMG-CoA) reductase inhibitors have been used with the aim of preventing coronary artery diseases, and demonstrated to have some benefits in many large scale clinical tests. However, their preventive effect on coronary diseases is limited to some extent, and is not satisfactory enough yet.

Recently, low serum level of high density lipoprotein (HDL) cholesterol has been revealed to be a potent risk factor of arteriosclerotic diseases by a number of epidemiological surveys and large scale clinical tests. HDL is known to have various antiarteriosclerotic effects and attention is focused on the potentiality of drugs increasing HDL cholesterol level as a means for prevention or treatment of arteriosclerotic diseases. However, there are no drugs that can be used in a satisfactory manner for this purpose. Fibrates and HMG-CoA reductase inhibitors have only low activity of increasing HDL cholesterol level; nicotinic acid derivatives can significantly increase HDL cholesterol level, but have serious toleration issues. Accordingly, there has been a demand for a well-tolerated agent which can significantly elevate HDL cholesterol level, thereby preventing or reversing the progression of atherosclerosis.

It is known that many proteins are involved in the regulation mechanism for catabolism of various lipoproteins. Among them, the role of cholesteryl ester transfer protein (CETP) became to draw attention. CETP is a protein responsible for transfer of cholesteryl ester (CE) and triglyceride between lipoproteins, and mediate the transfer of CE from HDL to LDL or to very low density lipoprotein (VLDL). Accordingly, CETP activity affects greatly the lipid composition in lipoprotein particles. For example, it is known that administration of an active neutralizing monoclonal antibody against CETP to rabbit or hamster elevates HDL cholesterol level and lower LDL cholesterol level. Furthermore, human being having decreased or eliminated CETP activity due to gene mutation shows raised blood HDL cholesterol level and lowered blood LDL cholesterol level. On the other hand, it is known that transgenic mice and rats made to express CETP show lowered HDL cholesterol level and raised LDL cholesterol level. Thus, it is considered that CETP greatly contribute to the regulation of serum lipids, and thereby affecting the change of serum lipid profile such as decrease of HDL cholesterol level and increase of LDL cholesterol level. Accordingly, it is assumed that a high value of CETP activity would induce arteriosclerosis.

In fact, CETP activity varies depending on animal species. It is known that, arteriosclerotic lesions are readily formed by cholesterol loading in animals with high CETP activity such as rabbits, whereas such lesions hardly occur in animals with low CETP activity such as rats. Furthermore, it is confirmed that continuous suppression of CETP activity by administration of antisense oligodeoxynucleotide resulted in effects such as increase of blood HDL cholesterol level and reduction in arteriosclerotic lesions in cholesterol-fed rabbits.

The above findings indicate that CETP activity is in negative correlation with HDL cholesterol, and that inhibition of CETP activity would decrease the degree of risk for arteriosclerotic diseases. It is therefore expected that compounds capable of inhibiting CETP activity can block the transfer of cholesterol from HDL to LDL or VLDL, and thereby increasing HDL cholesterol that tends to prevent arteriosclerosis while lowering LDL cholesterol that tends to promote arteriosclerosis. In this way, such compounds can serve as a useful preventive or therapeutic agent for arteriosclerotic diseases, hyperlipemia or dyslipidemia and provide effective medical treatment for the first time.

Examples of compounds having CETP inhibitory activity include tetrahydroquinoline derivatives. See, PCT International Publication WO00/17164 pamphlet, PCT International Publication WO00/17165 pamphlet and PCT International Publication WO00/17166 pamphlet.

However, these compounds have defects. That is, they are poorly soluble in water and cannot be absorbed enough in vivo, a sufficient blood level for taking medicinal effect can hardly be achieved even when administered as an ordinary formulation for oral administration. See, WO03/63868.

Accordingly, it has been demanded to find a novel compound which eliminates the above-mentioned defects and intensive studies have been done on dibenzylamine type compounds, and the like. See, PCT International Publication WO05/100298 pamphlet, PCT International Publication WO04/020393 pamphlet, PCT International Publication WO06/073973 pamphlet and JP 2003-221376 A.

### DISCLOSURE OF INVENTION

The present invention provides compounds having an excellent inhibitory activity against CETP, thereby useful for prophylaxis and/or treatment of arteriosclerotic diseases, hyperlipemia or dyslipidemia.

The present inventors have intensively studied in order to achieve the above-mentioned objects, and have found a compound having an inhibitory activity against CETP and showing an activity of increasing HDL cholesterol level and an activity of decreasing LDL cholesterol level, and have accomplished the present invention.

### BEST MODES FOR CARRYING OUT THE INVENTION

That is, the present invention provides the following embodiments:

1. A compound of the general formula (1):

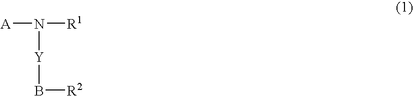

wherein, Y is a methylene group optionally substituted by a substituent(s) selected from an alkyl group and an oxo group, or a single bond;

A is (i) a group selected from an optionally substituted alkynyl group, a halogen atom, an oxo group, a hydroxy group, a cyano group, a nitro group, a carboxyl group, a sulfo group, an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, and a carbonyl group substituted by optionally substituted heterocyclic group;

(ii) a homocyclic group optionally substituted by 1 to 5 substituents selected independently from the groups as defined above in (i); or
(iii) a heterocyclic group optionally substituted by 1 to 5 substituents selected independently from the groups as defined above in (i);

B is a heterocyclic group optionally substituted by 1 to 5 substituents selected independently from the following groups: an optionally substituted alkynyl group, a halogen atom, an oxo group, a hydroxy group, a cyano group, a nitro group, a carboxyl group, a sulfo group, an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, a carbonyl group substituted by optionally substituted heterocyclic group, and an alkylene group; wherein said alkylene group may have 1 to 3 heteroatoms selected independently from oxygen, sulfur and nitrogen atoms and further optionally may have a substituent(s);

R¹ is a hydrogen atom or an optionally substituted alkyl group; wherein the alkyl group further may optionally be substituted by a substituent(s) selected from an optionally substituted homocyclic group and an optionally substituted heterocyclic group;

R² is a group selected from an optionally substituted alkynyl group, a halogen atom, an oxo group, a hydroxy group, a cyano group, a nitro group, a carboxyl group, a sulfo group, an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, and a carbonyl group substituted by optionally substituted heterocyclic group,

or a pharmaceutically acceptable derivative thereof.
2. The compound of the above embodiment 1 wherein the homocyclic group is a cycloalkyl group, a phenyl group or a naphthyl group;

the heterocyclic group is a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl or tetrahydroquinolyl group;

a substituent(s) for an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, a carbonyl group substituted by optionally substituted heterocyclic group, an optionally substituted phenyl group, an optionally substituted alkylsulfonyloxy group, an optionally substituted alkynyl group or an optionally substituted alkylene group is/are 1 to 5 groups selected independently from the following groups:

a halogen atom; a cyano group; a hydroxy group; a nitro group; a carboxyl group; an oxo group; a thioxo group; a sulfo group; a cycloalkyl group optionally substituted by hydroxy group, halogen atom, carboxyl group, alkoxycarbonyl group, mono- or di-alkylamino group, phenyl group or morpholinyl group; an alkoxycarbonyl group optionally substituted by hydroxy group, halogen atom, carboxyl group, alkoxycarbonyl group, mono- or di-alkylamino group, phenyl group or morpholinyl group; a carbamoyl group; a mono- or di-alkylcarbamoyl group optionally substituted by hydroxy group, halogen atom, carboxyl group, alkoxycarbonyl group, mono- or di-alkylamino group, phenyl group or morpholinyl group; an alkyl group optionally substituted by hydroxy group, halogen atom, carboxyl group, alkoxycarbonyl group, mono- or di-alkylamino group, phenyl group or morpholinyl group; an alkanoyl group optionally substituted by hydroxy group, halogen atom, carboxyl group, alkoxycarbonyl group, mono- or di-alkylamino group, phenyl group or morpholinyl group; an alkoxy group optionally substituted by hydroxy group, halogen atom, carboxyl group, alkoxycarbonyl group, mono- or di-alkylamino group, phenyl group or morpholinyl group; an alkanoyloxy group optionally substituted by hydroxy group, halogen atom, carboxyl group, alkoxycarbonyl group, mono- or di-alkylamino group, phenyl group or morpholinyl group; an alkylsulfanyl group optionally substituted by hydroxy group, halogen atom, carboxyl group, alkoxycarbonyl group, mono- or di-alkylamino group, phenyl group or morpholinyl group; an alkylsulfonyl group optionally substituted by hydroxy group, halogen atom, carboxyl group, alkoxycarbonyl group, mono- or di-alkylamino group, phenyl group or morpholinyl group; an alkylsulfinyl group optionally substituted by hydroxy group, halogen atom, carboxyl group, alkoxycarbonyl group, mono- or di-alkylamino group, phenyl group or morpholinyl group; a mono- or di-alkylsulfamoyl group optionally substituted by hydroxy group, halogen atom, carboxyl group, alkoxycarbonyl group, mono- or di-alkylamino group, phenyl group or morpholinyl group; an amino group; a mono- or di-alkylamino group optionally substituted by hydroxy group, halogen atom, carboxyl group, alkoxycarbonyl group, mono- or di-alkylamino group, phenyl group or morpholinyl group; a mono- or di-alkylsulfamoylamino group optionally substituted by hydroxy group, halogen atom, carboxyl group, alkoxycarbonyl group, mono- or di-alkylamino group, phenyl group or morpholinyl group; a mono- or di-alkylureido group optionally substituted by hydroxy group, halogen atom, carboxyl group, alkoxycarbonyl group, mono- or di-alkylamino group, phenyl group or morpholinyl group; a homocyclic group selected from a cycloalkyl group, a phenyl group and a naphthyl group; an oxy group substituted by the homocyclic group as defined above; a carbonyl group substituted by the homocyclic group as defined above; a heterocyclic group selected from a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl and tetrahydroquinolyl groups; an oxy group substituted by the heterocyclic group as defined above; a carbonyl group substituted by the heterocyclic group as defined above; and a group of the formulae:

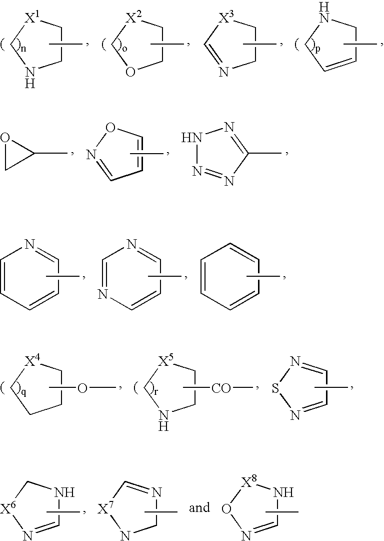

wherein X¹ and X³ are each independently CH₂, NH, O, S, SO or SO₂; X² and X⁵ are each independently CH₂, O, S, SO or SO₂; X⁴ is NH, O, S, SO or SO₂; X⁶ and X⁷ are each independently O or S; X⁸ is S or SO; and n, o, p, q and r are each independently an integer of 1 to 4, and further each of the above groups may optionally be substituted by 1 to 3 substituents selected from the following groups: halogen atom, carboxyl group, hydroxy group, cyano group, oxo group, thioxo group, alkyl group, hydroxyalkyl group, alkoxycarbonylalkyl group, carboxyalkyl group, morpholinylalkyl group, phenylalkyl group, alkanoyl group, hydroxyalkanoyl group, alkoxyalkanoyl group, alkoxy group, phenylalkoxy group, alkoxycarbonyl group, benzyloxycarbonyl group, mono- or di-alkylamino group, mono- or di-alkylcarbamoyl group, mono- or di-alkylsulfamoyl group, alkylsulfonyl group and tetrazolyl group-,
or a pharmaceutically acceptable derivative thereof.
3. The compound of the above embodiment 2 wherein A is a group of a formula:

-A¹-A²;

wherein A¹ is a phenyl, naphthyl, pyrimidinyl, pyridazinyl, pyridyl, triazolyl, tetrazolyl, oxadiazolyl, dihydropyrimidinyl, pyrazinyl, thiazolyl, oxazolyl, dihydrooxazinyl, imidazolyl, pyrazolyl or dihydropyrazinyl group;

A² is a carboxyl group; a cyano group; a nitro group; an alkyl group optionally substituted by a group selected from a hydroxy group, a cyano group, a carboxyl group, an alkoxycarbonyl group, an alkoxy group, a phenylalkoxy group, a hydroxyalkoxy group, a carboxyalkoxy group, an alkylsulfanyl group, an alkylsulfonyl group, an alkylsulfinyl group, an amino group, a mono- or di-alkylamino group, a mono- or di-alkylsulfamoylamino group, a mono- or di-alkylureido group optionally substituted by morpholinyl group, an oxiranyl group, a dialkyldioxolanyl group, a pyrrolidinyl group optionally substituted by carboxyl group, a piperidyl group optionally substituted by carboxyl group, a piperazinyl group optionally substituted by alkyl group, and a morpholinyl group; an alkenyl group optionally substituted by carboxyl group; an alkoxy group optionally substituted by a group selected from a hydroxy group, a cyano group, a carboxyl group, an alkoxycarbonyl group, an alkoxy group, a phenylalkoxy group, a hydroxyalkoxy group, a carboxyalkoxy group, an alkylsulfanyl group, an alkylsulfonyl group, an alkylsulfinyl group, an amino group, a mono- or di-alkylamino group, a mono- or di-alkylsulfamoylamino group, a mono- or di-alkylureido group optionally substituted by morpholinyl group, an oxiranyl group, a dialkyldioxolanyl group, a pyrrolidinyl group optionally substituted by carboxyl group, a piperidyl group optionally substituted by carboxyl group, a piperazinyl group optionally substituted by alkyl group, and a morpholinyl group; an alkoxycarbonyl group; a hydroxycarbamimidoyl group; an alkylsulfanyl group; an alkylsulfonyl group optionally substituted by carboxyl group; a mono- or di-alkylamino group optionally substituted by hydroxy group, carboxyl group, alkoxy group or mono- or di-alkylamino group; a morpholinyl group optionally substituted by carboxyl group, alkyl group, carboxyalkyl group or alkoxycarbonyl group; an optionally oxidized thiomorpholinyl group; a piperazinyl group optionally substituted by a group selected from an alkyl group, alkanoyl group and hydroxyalkanoyl group; a pyrrolidinyl group optionally substituted by carboxyl group, alkyl group, carboxyalkyl group or alkoxycarbonyl group; a piperidyl group optionally substituted by carboxyl group, alkyl group, carboxyalkyl group or alkoxycarbonyl group; a tetrazolyl group optionally substituted by alkyl group, hydroxyalkyl group, carboxyalkyl group or morpholinylalkyl group; an oxodihydrooxadiazolyl group; a pyrimidinyl group; or a tetrahydropyranyl group;

R¹ is a group of a formula:

—R¹¹R¹²,

wherein R¹¹ is an alkylene group;

R¹² is a substituent(s) selected from a phenyl, pyridyl, pyrimidinyl, pyridazinyl, furyl, thienyl, triazolyl, tetrazolyl, oxadiazolyl, dihydropyrimidinyl, pyrazinyl, thiazolyl, oxazolyl, imidazolyl, dihydrooxazinyl, dihydropyrazinyl and pyrazolyl group;

wherein said substituent(s) may optionally be substituted by 1 to 4 substituents selected independently from halogen atom, carboxyl group, alkoxycarbonyl group, carbamoyl group, mono- or di-alkylcarbamoyl group, alkyl group, alkoxy group, hydroxy group, nitro group, cyano group, amino group, mono- or di-alkylamino group, alkanoyl group, alkylsulfanyl group, tetrazolyl group and dihydrooxazolyl group; and further each of said alkyl group, alkoxy group, mono- or di-alkylamino group, mono- or di-alkylcarbamoyl group, alkanoyl group and alkylsulfanyl group independently may optionally be substituted by 1 to 5 substituents selected independently from halogen atom, hydroxy group, alkoxy group, amino group, morpholinyl group, piperidyl group, pyrrolidinyl group, piperazinyl group, alkylpiperazinyl group and alkanoylpiperazinyl group;

R² is a halogen atom;

a hydroxy group;
a cyano group;
a nitro group;
a carboxyl group;
a sulfo group;
a cycloalkyl group optionally substituted by carboxyl group or alkoxycarbonyl group;
an alkyl group optionally substituted by a group selected from a halogen atom, a cyano group, a hydroxy group, a carboxyl group, an alkoxycarbonyl group, a tetrazolyl group, a mono- or di-alkylcarbamoyl group, an alkoxy group (said alkoxy group may optionally be substituted by phenyl group, carboxyl group or hydroxy group), an alkanoyl group, an alkanoyloxy group, an alkylsulfanyl group, an alkylsulfonyl group, an alkylsulfinyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, an oxiranyl group, a dioxolanyl group optionally substituted by alkyl group, pyrrolidinyl group optionally substituted by alkoxycarbonyl group or carboxyl group, a pyrrolidinyl group optionally substituted by alkoxycarbonylalkyl group or carboxyalkyl group, a piperidyl group optionally substituted by alkoxycarbonyl group or carboxyl group, a piperidyl group optionally substituted by alkoxycarbonylalkyl or carboxyalkyl group, a piperazinyl group optionally substituted by alkyl group, a hexahydroazepinyl group, a morpholinyl group, and a piperidyloxy group optionally substituted by alkyl group;
an alkenyl group optionally substituted by a group selected from a cyano group, a hydroxy group, a carboxyl group, a benzyloxycarbonyl group and a tetrazolyl group;
an alkenyloxy group optionally substituted by carboxyl group;
an alkoxy group optionally substituted by a group selected from a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group optionally substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;
an alkoxycarbonyl group optionally substituted by phenyl group;
a carbamoyl group;
a mono- or di-alkylcarbamoyl group optionally substituted by a group selected from a carboxyl group, a morpholinyl group and an alkoxy group;
a hydroxycarbamimidoyl group;
an alkylsulfanyl group optionally substituted by a group selected from hydroxy group, carboxyl group and mono- or di-alkylcarbamoyl group;
an alkylsulfinyl group;
an alkylsulfonyl group optionally substituted by a group selected from hydroxy group, carboxyl group, alkoxycarbonyl group and mono- or di-alkylcarbamoyl group;
an amino group;
a mono- or di-alkylamino group optionally substituted by a group selected from a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl or carboxyalkyl group, a pyrrolidinyl group substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl optionally substituted by oxo group, a pyrrolidinylcarbonyl group optionally substituted by carboxyl group, a piperidyloxy group optionally substituted by alkyl group and a morpholinylcarbonyl group;
an alkanoylamino group optionally substituted by a group selected from hydroxy group, alkoxy group, carboxyl group and amino group;
a mono- or di-alkylcarbamoylamino group optionally substituted by alkoxy group;
a morpholinylcarbonylamino group;
a sulfamoyl group;
a mono- or di-alkylsulfamoyl group;
an alkanoyl group optionally substituted by a group selected from hydroxy group, carboxyl group, alkoxycarbonyl group, alkoxy group, mono- or di-alkylamino group and morpholinyl group; or
a cyclic group selected from a cycloalkyl, phenyl, naphthyl, thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl and tetrahydroquinolyl groups;
wherein said cyclic group may optionally be substituted by the following groups: a halogen atom, an alkoxyalkyl group, an alkyl group optionally substituted by 1 to 5 halogen atoms, a mono- or di-alkylaminoalkyl group, a mono- or di-alkylaminoalkoxy group, a carboxyl group, a hydroxy group, a cyano group, an oxo group, an alkyl group, a hydroxyalkyl group, an alkoxycarbonylalkyl group, a carboxyalkyl group, a morpholinylalkyl group, a phenylalkyl group, an alkanoyl group, a hydroxyalkanoyl group, an alkoxyalkanoyl group, an alkoxy group, a phenylalkoxy group, an alkoxycarbonyl group, a benzyloxycarbonyl group, a mono- or di-alkylamino group, a mono- or di-alkylcarbamoyl group, a mono- or di-alkylsulfamoyl group, an alkylsulfonyl group and a tetrazolyl group;
wherein the substituents defined as above may further be substituted by a substituent(s) selected from the following groups:

a halogen atom, an alkoxyalkyl group, an alkyl group optionally substituted by 1 to 5 halogen atoms, a mono- or di-alkylaminoalkyl group, a mono- or di-alkylaminoalkoxy group, a carboxyl group, a hydroxy group, a cyano group, an oxo group, an alkyl group, a hydroxyalkyl group, an alkoxycarbonylalkyl group, a carboxyalkyl group, a morpholinylalkyl group, a phenylalkyl group, an alkanoyl group, a hydroxyalkanoyl group, an alkoxyalkanoyl group, an alkoxy group, a phenylalkoxy group, an alkoxycarbonyl group, a benzyloxycarbonyl group, a mono- or di-alkylamino group, a mono- or di-alkylcarbamoyl group, a mono- or di-alkylsulfamoyl group, an alkylsulfonyl group and a tetrazolyl group,

or a pharmaceutically acceptable derivative thereof.
4. The compound of the above embodiment 1 wherein Y is a methylene group optionally substituted by a group(s) selected from an alkyl group and an oxo group, or a single bond;

A is a group of a formula:

-A¹-A²;

wherein A¹ is a homocyclic group, a heterocyclic group or a single bond;

A² is an optionally substituted homocyclic group, an optionally substituted alkylsulfanyl group, an optionally substituted alkylsulfinyl group, an optionally substituted alkylsulfonyl group, a nitro group, a hydroxy group, an optionally substituted alkenyl group, an optionally substituted heterocyclic group, an optionally substituted alkoxy group, a halogen atom, an optionally substituted alkyl group, a cyano group, an amino group optionally substituted by 1 to 2 substituents, or a hydrogen atom;

B is a heterocyclic group optionally substituted by 1 to 5 groups selected independently from an oxo group, a cyano group, a carboxyl group, an optionally substituted alkoxycarbonyl group, an optionally substituted carbamoyl group, a hydroxyl group, an optionally substituted alkylsulfonyl group, an optionally substituted alkyl group, a halogen atom, an optionally substituted alkoxy group, an optionally substituted alkylsulfanyl group, an optionally substituted cycloalkyl group, an optionally substituted cycloalkoxy group, an optionally substituted heterocyclic group, an optionally substituted alkylsulfinyl group and an amino group optionally substituted by 1 to 2 substituents;

R¹ is a hydrogen atom or an optionally substituted lower alkyl group; said lower alkyl group further may optionally be substituted by an optionally substituted homocyclic group or an optionally substituted heterocyclic group;

R² is an optionally substituted alkylsulfanyl group, an optionally substituted alkylsulfinyl group, an optionally substituted alkylsulfonyl group, a cyano group, an optionally substituted alkenyl group, an amino group optionally substituted by 1 to 2 substituents, a halogen atom, an optionally substituted alkoxy group, an optionally substituted carbamoyl group, an oxy group substituted by optionally substituted heterocyclic group, a hydroxy group, an optionally substituted heterocyclic group, an optionally substituted homocyclic group, an oxy group substituted by optionally substituted homocyclic group, an optionally substituted alkyl group, a hydrogen atom, an optionally substituted alkylcarbonyl group, an optionally substituted alkyl group, an optionally substituted alkoxycarbonyl group or a nitro group;

provided that a partial structure (1-1):

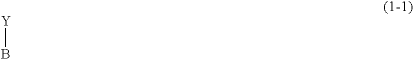

wherein the symbols have the same meanings as defined above, in the compound (1) is not a formula:

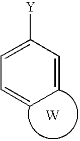

wherein a cyclic W is a heterocyclic group,
or a pharmaceutically acceptable derivative thereof.
5. The compound of the above embodiment 4 wherein Y is a methylene group optionally substituted by a group(s) selected independently from an alkyl group and an oxo group, or a single bond;

A¹ is a heterocyclic group or a single bond;

A² is an optionally substituted heterocyclic group, an optionally substituted alkoxy group, a halogen atom, an optionally substituted alkyl group, a cyano group, an amino group optionally substituted by 1 to 2 substituents, or a hydrogen atom;

B is a heterocyclic group optionally substituted by 1 to 5 groups selected independently from an oxo group, a cyano group, a carboxyl group, an optionally substituted alkoxycarbonyl group, an optionally substituted carbamoyl group, a hydroxyl group, an optionally substituted alkylsulfonyl group, an optionally substituted alkyl group, a halogen atom, an optionally substituted alkoxy group, an optionally substituted alkylsulfanyl group, an optionally substituted alkylsulfinyl group, an optionally substituted piperidyl group, and an amino group optionally substituted by 1 to 2 substituents;

R¹ is a hydrogen atom, an optionally substituted benzyl group or an optionally substituted phenylethyl group;

R² is an amino group optionally substituted by 1 to 2 substituents, a halogen atom, an optionally substituted alkoxy group, an optionally substituted carbamoyl group, an oxy group substituted by optionally substituted heterocyclic group, a hydroxy group, an optionally substituted heterocyclic group, an optionally substituted homocyclic group, an oxy group substituted by optionally substituted homocyclic group, a hydroxyalkyl group or a nitro group,

or a pharmaceutically acceptable derivative thereof.
6. The compound of the above embodiment 5 wherein Y is a methylene group optionally substituted by a group(s) selected independently from an alkyl group and an oxo group, or a single bond;

A¹ is a heterocyclic group or a single bond;

A² is an optionally substituted heterocyclic group, an optionally substituted alkoxy group, a halogen atom, an optionally substituted alkyl group, a cyano group, an amino group optionally substituted by 1 to 2 substituents, or a hydrogen atom;

B is a heterocyclic group optionally substituted by 1 to 5 group selected independently from an optionally substituted alkyl group, a halogen atom, a hydroxy group, an optionally substituted cycloalkyl group, an optionally substituted cycloalkoxy group, an optionally substituted alkoxy group, an alkylsulfanyl group, an alkylsulfinyl group, an amino group optionally substituted by 1 to 2 substituents and an alkylsulfonyl group;

R¹ is

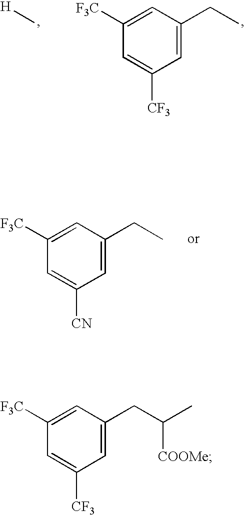

R² is an amino group optionally substituted by 1 to 2 substituents, a halogen atom, an optionally substituted alkoxy group, an optionally substituted carbamoyl group, an oxy group substituted by optionally substituted heterocyclic group, a hydroxy group, an optionally substituted heterocyclic group, an optionally substituted homocyclic group, an oxy group substituted by optionally substituted homocyclic group, an optionally substituted alkyl group, a hydrogen atom, an optionally substituted alkylcarbonyl group, an optionally substituted alkoxycarbonyl group, or a nitro group,

or a pharmaceutically acceptable derivative thereof.
7. The compound of the above embodiment 6 wherein the homocyclic group is a cycloalkyl group, a phenyl group or a naphthyl group;

the heterocyclic group is a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl or tetrahydroquinolyl group;

a substituent(s) for an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, a carbonyl group substituted by optionally substituted heterocyclic group, an optionally substituted phenyl group, an optionally substituted alkylsulfonyloxy group, an optionally substituted alkynyl group or an optionally substituted alkylene group is/are 1 to 5 groups selected independently from the following groups:

a halogen atom; a cyano group; a hydroxy group; a nitro group; a carboxyl group; an oxo group; a thioxo group; a sulfo group; a cycloalkyl group optionally substituted by hydroxy group, halogen atom, carboxyl group, alkoxycarbonyl group, mono- or di-alkylamino group, phenyl group or morpholinyl group; an alkoxycarbonyl group optionally substituted by hydroxy group, halogen atom, carboxyl group, alkoxycarbonyl group, mono- or di-alkylamino group, phenyl group or morpholinyl group; a carbamoyl group; a mono- or di-alkylcarbamoyl group optionally substituted by hydroxy group, halogen atom, carboxyl group, alkoxycarbonyl group, mono- or di-alkylamino group, phenyl group or morpholinyl group; an alkyl group optionally substituted by hydroxy group, halogen atom, carboxyl group, alkoxycarbonyl group, mono- or di-alkylamino group, phenyl group or morpholinyl group; an alkanoyl group optionally substituted by hydroxy group, halogen atom, carboxyl group, alkoxycarbonyl group, mono- or di-alkylamino group, phenyl group or morpholinyl group; an alkoxy group optionally substituted by hydroxy group, halogen atom, carboxyl group, alkoxycarbonyl group, mono- or di-alkylamino group, phenyl group or morpholinyl group; an alkanoyloxy group optionally substituted by hydroxy group, halogen atom, carboxyl group, alkoxycarbonyl group, mono- or di-alkylamino group, phenyl group or morpholinyl group; an alkylsulfanyl group optionally substituted by hydroxy group, halogen atom, carboxyl group, alkoxycarbonyl group, mono- or di-alkylamino group, phenyl group or morpholinyl group; an alkylsulfonyl group optionally substituted by hydroxy group, halogen atom, carboxyl group, alkoxycarbonyl group, mono- or di-alkylamino group, phenyl group or morpholinyl group; an alkylsulfinyl group optionally substituted by hydroxy group, halogen atom, carboxyl group, alkoxycarbonyl group, mono- or di-alkylamino group, phenyl group or morpholinyl group; a mono- or di-alkylsulfamoyl group optionally substituted by hydroxy group, halogen atom, carboxyl group, alkoxycarbonyl group, mono- or di-alkylamino group, phenyl group or morpholinyl group; an amino group; a mono- or di-alkylamino group optionally substituted by hydroxy group, halogen atom, carboxyl group, alkoxycarbonyl group, mono- or di-alkylamino group, phenyl group or morpholinyl group; a mono- or di-alkylsulfamoylamino group optionally substituted by hydroxy group, halogen atom, carboxyl group, alkoxycarbonyl group, mono- or di-alkylamino group, phenyl group or morpholinyl group; a mono- or di-alkylureido group optionally substituted by hydroxy group, halogen atom, carboxyl group, alkoxycarbonyl group, mono- or di-alkylamino group, phenyl group or morpholinyl group; a homocyclic group selected from a cycloalkyl group, a phenyl group and a naphthyl group; an oxy group substituted by the homocyclic group as defined above; a carbonyl group substituted by the homocyclic group as defined above; a heterocyclic group selected from a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl and tetrahydroquinolyl groups; an oxy group substituted by the heterocyclic group as defined above; a carbonyl group substituted by the heterocyclic group as defined above; and a group of the formulae:

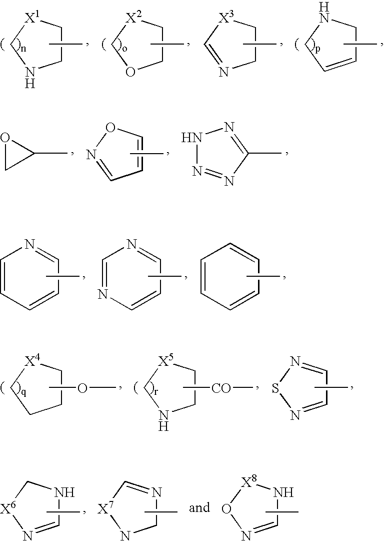

wherein X¹ and X³ are each independently CH₂, NH, O, S, SO or SO₂; X² and X⁵ are each independently CH₂, O, S, SO or SO₂; X⁴ is NH, O, S, SO or SO₂; X⁶ and X⁷ are each independently O or S; X⁸ is S or SO; and n, o, p, q and r are each independently an integer of 1 to 4, and further each of the above groups may optionally be substituted by 1 to 3 substituents selected from the following groups: halogen atom, carboxyl group, hydroxy group, cyano group, oxo group, thioxo group, alkyl group, hydroxyalkyl group, alkoxycarbonylalkyl group, carboxyalkyl group, morpholinylalkyl group, phenylalkyl group, alkanoyl group, hydroxyalkanoyl group, alkoxyalkanoyl group, alkoxy group, phenylalkoxy group, alkoxycarbonyl group, benzyloxycarbonyl group, mono- or di-alkylamino group, mono- or di-alkylcarbamoyl group, mono- or di-alkylsulfamoyl group, alkylsulfonyl group and tetrazolyl group,
or a pharmaceutically acceptable derivative thereof.
8. The compound of the above embodiment 7 wherein Y is a methylene group or a single bond;

A¹ is a pyrimidinyl group, a tetrazolyl group or a single bond;

A² is a mono- or di-alkylamino group optionally substituted by carboxyl group, a piperidyl group optionally substituted by carboxyl group, a morpholinyl group optionally substituted by carboxyl group, an alkoxy group optionally substituted by carboxyl group, a halogen atom, an alkyl group optionally substituted by carboxyl group, a hydrogen atom or a cyano group;

B is an isoquinolyl, tetrahydroquinolyl, quinolyl, pyrazolyl, pyridyl, pyrimidinyl, dihydroindolyl, imidazolyl or imidazopyridyl group; wherein said heterocyclic group may optionally be substituted by 1 to 5 substituents selected independently from the following groups: an oxo group; an alkoxy group optionally substituted by 1 to 3 halogen atoms; an alkyl group optionally substituted by 1 to 3 halogen atoms; a halogen atom; a hydroxy group; an alkylsulfanyl group; an alkylsulfinyl group; and an amino group optionally substituted by 1 to 2 substituents selected independently from an alkyl group or an alkyl group substituted by morpholinyl;

R¹ is a hydrogen atom, or a benzyl group substituted by 1 to 3 groups selected independently from an alkoxy group optionally substituted by cyano group and 1 to 3 halogen atoms and an alkyl group optionally substituted by 1 to 3 halogen atoms;

R² is (a) an amino group optionally substituted by 1 to 2 groups independently selected from an alkyl group, an alkoxyalkyl group, a cycloalkylalkyl group, an alkoxycarbonyl group, an alkylcarbonyl group, an alkylcarbamoyl group, a carboxyalkyl group, a cycloalkylalkyl group substituted by carboxyalkyl group, a hydroxyalkyl group, a carboxyalkoxycarbonyl group, a carboxydihydrooxazolyl group, a carboxyalkylcarbonyl group, a phenylalkyl group, an alkoxyalkoxycarbonyl group, an alkoxyalkylcarbonyl group, an alkyl group substituted by piperidyl group, a piperidylalkyl group substituted by carboxyalkyl group, and an alkyl group substituted by phenyl group that is optionally substituted by 1 to 2 alkyl groups (said alkyl group may optionally be substituted by 1 to 3 halogen atoms);

wherein said alkyl group or alkoxy group may further optionally be substituted by 1 to 5 groups selected independently from the following groups:

a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group optionally substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;

(b) a halogen atom;

(c) an alkoxy optionally substituted by a group selected from carboxyl group, cycloalkyl group and alkoxy group;

wherein said cycloalkyl group or alkoxy group may optionally be substituted by 1 to 5 groups selected independently from the following groups:

a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group optionally substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;

(d) a carbamoyl group optionally substituted by 1 to 2 substituents selected independently from alkyl group and carboxyalkyl group, wherein said alkyl group may optionally be substituted by 1 to 5 groups selected independently from the following groups:

a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may be substituted by carboxyl, alkoxycarbonyl or hydroxy group), an alkoxy group (said alkoxy group may be substituted by carboxyl, formyl or hydroxy group), an alkanoyloxy group, an alkylsulfanyl group, an alkylsulfonyl group, an alkylsulfinyl group, an aminosulfonyl group, an amino group, a mono- or di-alkylamino group-optionally substituted by carboxyl or alkoxy group, a mono- or di-alklsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy or carboxyl group, a morpholinyl group, a pyrrolidinyl optionally substituted by alkoxycarbonyl or carboxyl group, a pyrrolidinyl optionally substituted by alkoxycarbonylalkyl or carboxyalkyl group, a pyrrolidinyl substituted by oxo group, a piperidyl optionally substituted by alkoxycarbonyl or carboxyl group, a piperidyl optionally substituted by alkoxycarbonylalkyl or carboxyalkyl group, a piperazinyl optionally substituted by alkyl group, a hexahydroazepinyl group, a pyrimidinyl group, a pyridyl group, a dioxolany group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;

(e) a hydroxy group;

(f) an oxy group substituted by a heterocyclic group selected from pyrimidinyl group and tetrahydropyranyl group;

(g) a heterocyclic group selected from a morpholinyl, piperidyl, piperazinyl, pyrazinyl, tetrazolyl, thienyl, furyl, dihydroisoquinolyl, pyridyl and pyrrolyl group, which are each optionally substituted by 1 to 2 substituents selected independently from pyrimidinyl group, halogen atom, alkyl group, cyano group, mono- or di-alkylamino group, alkoxy group, phenyl group, carboxyl group, carbamoyl group and carboxyalkyl group; wherein said alkoxy group or alkyl group may optionally be substituted by 1 to 5 groups selected independently from the following groups:

a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;

(h) a phenyl group optionally substituted by 1 to 3 substituents selected independently from halogen atom, alkyl group and alkoxy group; wherein said alkoxy group or alkyl group may further optionally be substituted by 1 to 5 groups selected independently from the following groups:

a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;

(i) an oxy group substituted by cycloalkyl group;

(j) a hydroxyalkyl group; or

(k) a nitro group,

or a pharmaceutically acceptable derivative thereof.
9. The compound of the above embodiment 8 wherein Y is a methylene group or a single bond;

A¹ is a pyrimidinyl group, a tetrazolyl group or a single bond;

A² is a mono- or di-alkylamino group optionally substituted by carboxyl group, a piperidyl group optionally substituted by carboxyl group, a morpholinyl group optionally substituted by carboxyl group, alkoxy group optionally substituted by carboxyl group, a halogen atom, an alkyl group optionally substituted by carboxyl group, a hydrogen atom or a cyano group;

B is an isoquinolyl, tetrahydroquinolyl, quinolyl, pyrazolyl, pyridyl, pyrimidinyl, dihydroindolyl, imidazolyl or imidazopyridyl group; wherein said heterocyclic group may optionally be substituted by 1 to 5 substituents selected independently from the following groups: an oxo group; an alkoxy group optionally substituted by 1 to 3 halogen atoms; an alkyl group optionally substituted by 1 to 3 halogen atoms; a halogen atom; an alkylsulfanyl group; an alkylsulfinyl group; and an amino group optionally substituted by 1 to 2 substituents selected independently from an alkyl group or an alkyl group substituted by morpholinyl group;

R¹ is

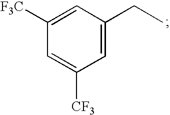

R² is (a) an amino group optionally substituted by 1 to 2 groups selected independently from an alkyl group, an alkoxyalkyl group, a cycloalkylalkyl group, an alkoxycarbonyl group, an alkylcarbonyl group, an alkylcarbamoyl group, a carboxyalkyl group, a cycloalkylalkyl group substituted by carboxyalkyl group, a hydroxyalkyl group, a carboxyalkoxycarbonyl group, a carboxydihydrooxazolyl group, a carboxyalkylcarbonyl group, a phenylalkyl group, an alkoxyalkoxycarbonyl group, an alkoxyalkylcarbonyl group, an alkyl group substituted by piperidyl group, a piperidylalkyl group substituted by carboxyalkyl group and an alkyl group substituted by phenyl group that is optionally substituted by 1 to 2 alkyl groups (said alkyl group may optionally be substituted by 1 to 3 halogen atoms);

(b) a halogen atom;

(c) an alkoxy group optionally substituted by a group selected from a carboxyl group, a cycloalkyl group and an alkoxy group;

(d) a carbamoyl group optionally substituted by 1 to 2 substituents selected independently from alkyl group and carboxyalkyl group;

(e) a hydroxy group;

(f) an oxy group substituted by a heterocyclic group selected from a pyrimidinyl group and a tetrahydropyranyl group;

(g) a heterocyclic group selected from a morpholinyl, piperidyl, pyrazinyl, dihydroisoquinolyl, pyridyl, pyrimidinyl, piperazinyl, tetrazolyl or pyrrolyl group, which are each optionally substituted by 1 to 3 substituents selected independently from alkyl group, halogen atom, phenyl group, alkoxy group, carboxyl group and carboxyalkyl group;

(h) a phenyl group optionally substituted by 1 to 3 substituents selected independently from halogen atom, alkyl group and alkoxy group;

(i) an oxy group substituted by cycloalkyl group;

(j) a hydroxyalkyl group;

(k) a nitro group,

or a pharmaceutically acceptable derivative thereof.
10. The compound of the above embodiment 9 wherein Y is a methylene group;

A¹ is a pyrimidinyl group;

A² is an alkoxy group optionally substituted by carboxyl group, a piperidyl group optionally substituted by carboxyl group, an amino group optionally substituted by 1 to 2 groups selected independently from carboxyalkyl group and alkyl group, or an alkyl group substituted by carboxyl group;

B is a pyrimidinyl, pyridyl, quinolyl, dihydroindolyl, pyrazolyl, isoquinolyl, imidazolyl or imidazopyridyl group; wherein said heterocyclic group may optionally be substituted by 1 to 5 substituents selected independently from the following groups: an oxo group; an alkoxy group optionally substituted by 1 to 3 halogen atoms; an alkyl group optionally substituted by 1 to 3 halogen atoms; a halogen atom; an alkylsulfanyl group; an alkylsulfinyl group; and an amino group optionally substituted by 1 to 2 substituents selected independently from an alkyl group or an alkyl group substituted by morpholinyl;

R¹ is

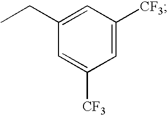

R² is (a) an amino group optionally substituted by 1 to 2 groups selected independently from a carboxyalkyl group, an alkoxyalkyl group, a hydroxyalkyl group, mono- or di-alkylaminoalkyl group, an alkyl group, a cycloalkylalkyl group and an alkoxycarbonyl group;

(b) an alkoxy group;

(c) a phenyl group optionally substituted by 1 to 2 substituents selected independently from an alkyl group and an alkoxy group;

(d) a pyrimidinyl group optionally substituted by 1 to 2 substituents selected from an alkoxy group and an alkyl group;

(e) a pyridyl group optionally substituted by 1 to 2 substituents selected from an alkoxy group and an alkyl group,

or a pharmaceutically acceptable derivative thereof.
11. A compound described in any one of examples No. 1, 4, 5, 14, 21 and 23, or a pharmaceutically acceptable derivative thereof.
12. A compound of the formula (I-A):

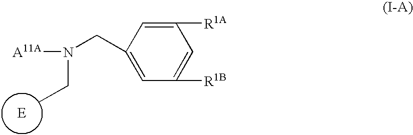

wherein

A^{11A} is an optionally substituted pyrimidin-2-yl group;

R^{1A} and R^{1B} are independently a cyano group or an alkyl group optionally substituted by 1 to 3 halogen atoms;

Ring E is an optionally substituted heterocyclic group which contain 1 to 2 nitrogen atoms,

or a pharmaceutically acceptable derivative thereof.
13. A compound of the formula (I-B):

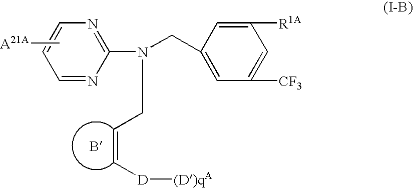

wherein

A^{21A} is an optionally substituted carbamoyl group, an optionally substituted homocyclic group, an optionally substituted alkylsulfanyl group, an optionally substituted alkylsulfinyl group, an optionally substituted alkylsulfonyl group, an optionally substituted alkyl group, a nitro group, a hydroxy group, a cyano group, an optionally substituted alkenyl group, an optionally substituted heterocyclic group, an optionally substituted alkoxy group, a halogen atom, an amino group optionally substituted by 1 to 2 substituents, a carboxyl group or a hydrogen atom; R^{1A} is a cyano group or an alkyl group optionally substituted by 1 to 3 halogen atoms;

B′ is optionally substituted by 1 to 3 groups selected independently from an oxo group, a cyano group, a halogen atom, a hydroxy group, an optionally substituted cycloalkyl group, an optionally substituted cycloalkoxy group, an optionally substituted heterocyclic group, an optionally substituted alkylsulfanyl group, an optionally substituted alkylsulfinyl group, an optionally substituted alkylsulfonyl group, an amino group optionally substituted by 1 to 2 substituents, an optionally substituted alkyl group and an optionally substituted alkoxy group;

D is a phenyl group, a tetrazol-5-yl group, a pyrimidin-2-yl group, a pyrimidin-4-yl group, a pyrimidin-5-yl group, a pyrimidin-6-yl group, a pyridin-2-yl group, a pyridin-3-yl group, a pyridin-4-yl group, a pyridin-5-yl group, a pyridin-6-yl group, or a hydrogen atom;

D′ is a group selected independently from a halogen atom, an alkoxyalkyl group, an alkyl group substituted by 1 to 5 halogen atoms, an alkoxy group substituted by 1 to 5 halogen atoms, an alkenyl group, a carbamoyl group, a cycloalkyl group, a mono- or di-alkylaminoalkyl group, a mono- or di-alkylaminoalkoxy group, a carboxyl group, a hydroxy group, a cyano group, an oxo group, an alkyl group, a hydroxyalkyl group, an alkoxycarbonylalkyl group, a carboxyalkyl group, a morpholinylalkyl group, a phenylalkyl group, an alkanoyl group, a hydroxyalkanoyl group, an alkoxyalkanoyl group, an alkoxy group, a phenylalkoxy group, an alkoxycarbonyl group, a benzyloxycarbonyl group, a mono- or di-alkylamino group, a mono- or di-alkylcarbamoyl group, a mono- or di-alkylsulfamoyl group, an alkylsulfonyl group, a tetrazolyl group, a benzyloxyalkyl group, a cycloalkylalkyl group, a benzyloxy group, an alkoxyalkoxy group, a carboxyalkoxy group, a carboxyalkenyl group, an alkylcarbonylamino group, a carboxyalkoxyalkyl group, a morpholinyl group or a pyridylalkoxy group;

q^{A} is an integer of 0 to 3;

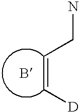

is a group selected from the following groups:

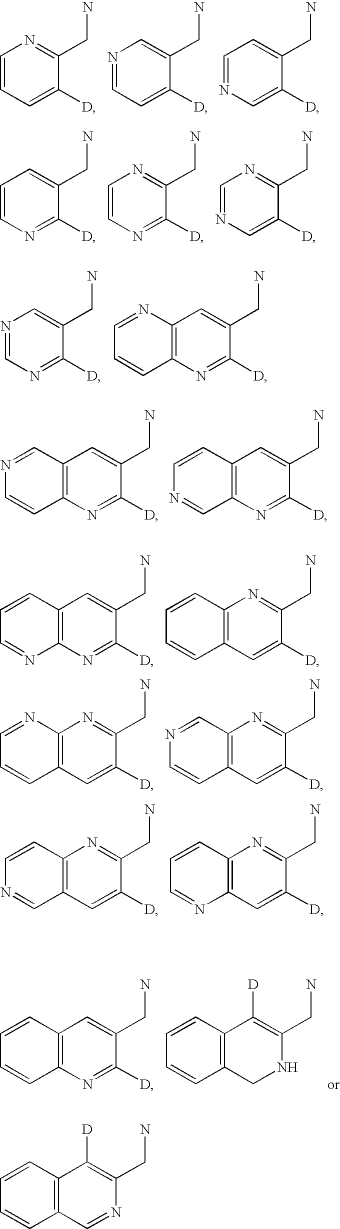

, or a pharmaceutically acceptable derivative thereof.
14. The compound of the above embodiment 13 wherein

A^{21A} is selected from the following group

(a) a heterocyclic group selected from a piperidyl group and a morpholinyl group, respectively optionally substituted by a substituent(s) selected from a carboxyl group, an alkoxycarbonyl group, a carboxyalkyl group or an alkyl group;

(b) an alkoxy group optionally substituted by a group selected from a carboxyl group, an alkoxycarbonyl group, a halogen atom, an alkylsulfinyl group, a mono- or di-alkylamino group, a cyano group, a tetrazolyl group, an alkylsulfonyl group, an alkylsulfanyl group, a hydroxy group or an alkoxy group;

(c) a halogen atom;

(d) an amino group optionally substituted by 1 to 2 substituents independently selected from a carboxyalkyl group, an alkoxycarbonylalkyl group, an alkylsulfonylalkyl group, an alkylsulfinylalkyl group, a hydroxyalkyl group, an alkyl group, an alkoxyalkyl group or an aminoalkyl group optionally substituted by 1 to 2 alkyl groups;

(e) a hydrogen atom;

(f) an alkyl group optionally substituted by a group selected from a carboxyl group, an alkoxycarbonyl group, a halogen atom, an alkylsulfinyl group, a mono- or di-alkylamino group, a cyano group, a tetrazolyl group, an alkylsulfonyl group, an alkylsulfanyl group, a hydroxy group or an alkoxy group;

(g) a carboxyl group;

(h) a carbamoyl group optionally substituted by a carboxyalkyl group;

(i) an alkenyl group substituted by a group selected from a carboxyl group, an alkoxycarbonyl group, an alkylsulfinyl group, a cyano group, a tetrazolyl group, an alkylsulfonyl group, an alkylsulfanyl group, a hydroxy group or an alkoxy group;

(j) a morpholinyl group;

(k) a piperidinyl group optionally substituted by a carboxyl group or a carboxyalkyl group;

B′ is optionally substituted by 1 to 3 groups selected independently from an oxo group, a halogen atom, an alkyl group optionally substituted by 1 to 3 halogen atoms, an alkoxy group optionally substituted by 1 to 3 halogen atoms, a cyano group, an alkylsulfanyl group optionally substituted by 1 to 3 halogen atoms, an alkylsulfinyl group optionally substituted by 1 to 3 halogen atoms, an amino group, an alkoxyalkyl group, a benzyloxy group, an alkoxyalkoxy group, a mono or di-alkylaminoalkoxy group, a mono or di-alkylaminoalkyl group, a cycloalkyl group, a cycloalkoxy group and an alkylsulfonyl group optionally substituted by 1 to 3 halogen atoms,

or a pharmaceutically acceptable derivative thereof.
15. The compound of the above embodiment 14 wherein

A^{21A} is a morpholinyl group, a carboxyalkoxy group, an alkylsulfonylalkoxy group, a carboxyalkenyl group, a carboxyalkyl group, an alkoxycarbonylalkoxy group, an alkoxycarbonylalkyl group or a carboxypiperidinyl group;

B′ is optionally substituted by 1 to 3 groups selected independently from an alkyl group optionally substituted by 1 to 3 halogen atoms, a halogen atom, an alkoxy group optionally substituted by 1 to 3 halogen atoms and an oxo group;

D′ is a group selected independently from a benzyloxyalkyl group, a halogen atom, a cycloalkylalkyl group, an alkyl group optionally substituted by 1 to 3 halogen atoms or an alkoxy group optionally substituted by 1 to 3 halogen atoms;

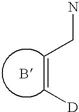

is a group selected from the following groups:

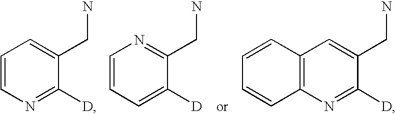

, or a pharmaceutically acceptable derivative thereof.
16. The compound of the above embodiment 15, wherein

A^{21A} is a carboxyalkoxy group or a carboxypiperidinyl group;

D is a phenyl group;

D′ is a group selected independently from a halogen atom, an alkoxy group or an alkyl group,

or a pharmaceutically acceptable derivative thereof.
17. The compound of the above embodiment 15, wherein

A^{21A} is a carboxyalkoxy group;

B′ is optionally substituted by 1 to 3 groups selected independently from an alkyl group optionally substituted by 1 to 3 halogen atoms and an alkoxy group optionally substituted by 1 to 3 halogen atoms;

D is a phenyl group;

D′ is a group selected independently from a halogen atom, an alkoxy group or an alkyl group;

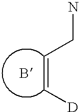

is

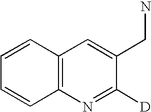

, or a pharmaceutically acceptable derivative thereof.
18. A compound described in any one of examples No. 52, 54, 55, 57, 58, 60, 63, 65, or a pharmaceutically acceptable derivative thereof.
19. A compound described in any one of examples No. 77 to 84, or a pharmaceutically acceptable derivative thereof.
20. A pharmaceutical composition, which comprises as an active ingredient a compound according to any one of the above embodiments 1 to 19, or a pharmaceutically acceptable derivative thereof.
21. A method for prophylaxis or treatment of arteriosclerosis such as atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular diseases, angina, ischemia, cardiac ischemia, stroke, myocardial infarction, reperfusion injury, restenosis after angioplasty, hypertension, cerebral infarction, cerebral stroke, diabetes, vascular complication of diabetes, thrombotic diseases, obesity, endotoxemia, metabolic syndrome, cerebrovascular disease, coronary artery disease, ventricular dysfunction, cardiac arrhythmia, pulmonary vascular disease, reno-vascular disease, renal disease, splanchnic vascular disease, vascular hemostatic disease, fatty liver disease, steatohepatitis, inflammatory disease, autoimmune disorders and other systemic disease indications, immune function modulation, pulmonary disease, anti-oxidant disease, sexual dysfunction, cognitive dysfunction, schistosomiasis, cancer, regression of xanthoma or Alzheimer's disease, which comprises administering an effective amount of a compound according to any one of the above embodiments 1 to 19, or a pharmaceutically acceptable derivative thereof, to a patient in need thereof.
22. Use of a compound according to any one of the above embodiments 1 to 19, or a pharmaceutically acceptable derivative thereof, for the manufacture of a medicament in treatment of patients suffering from arteriosclerosis such as atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular diseases, angina, ischemia, cardiac ischemia, stroke, myocardial infarction, reperfusion injury, restenosis after angioplasty, hypertension, cerebral infarction, cerebral stroke, diabetes, vascular complication of diabetes, thrombotic diseases, obesity, endotoxemia, metabolic syndrome, cerebrovascular disease, coronary artery disease, ventricular dysfunction, cardiac arrhythmia, pulmonary vascular disease, reno-vascular disease, renal disease, splanchnic vascular disease, vascular hemostatic disease, fatty liver disease, steatohepatitis, inflammatory disease, autoimmune disorders and other systemic disease indications, immune function modulation, pulmonary disease, anti-oxidant disease, sexual dysfunction, cognitive dysfunction, schistosomiasis, cancer, regression of xanthoma or Alzheimer's disease.
23. A compound of a general formula (1-2):

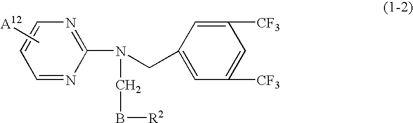

wherein the symbols have the same meanings as defined above, or a pharmaceutically acceptable derivative thereof.

When the compound (1) has one or more asymmetric carbon atoms, the compound (1) of the present invention encompasses racemate, racemic mixture, individual enantiomers or diastereomers. The present compounds also include all such isomers and a mixture thereof.

Also, when the present compound has an alkenyl or alkynyl group, cis (Z)- and trans (E)-forms may occur. In addition, the present compounds include individual stereoisomer of the compound and optionally, individual tautomeric forms thereof and a mixture thereof.

Separation of diastereomer or cis- and trans-isomers can be achieved by the conventional method such as fractional crystallization, chromatography and HPLC method, and the like. Also the drug containing the individual stereoisomer as needed can be prepared from a corresponding optically active intermediate or alternatively by resolving the corresponding racemate by using the suitable chiral support (e.g. HPLC) or by performing a fractional crystallization of diastereomeric salt formed by reacting a corresponding racemate and a suitable optically active acid or base. Alternatively, the resolution of the mixture of enantiomers can be done by forming on a novel covalently bounded species formed by means of reacting it with a suitable chiral compound. For example, at first, the coupling reaction between a racemic carboxylic acid and a chiral amine or a chiral alcohol gives a mixture of diastereoisomers (amide or ester, respectively) and then it is isolated by the conventional technique such as chromatography, HPLC or fractional crystallization, and the like. Thereafter, the resulting one diastereoisomer can be converted into one enantiomer of the desired compound by cleaving the new covalent binding with a suitable chemical reaction such as hydrolysis, and the like.

As used herein, the term “pharmaceutically acceptable derivative” represents a pharmaceutical acceptable salt, solvate or prodrug (e.g. ester) of the present compound, and which can provide (directly or indirectly) the compound of the present invention or an active metabolite or a residue thereof. Such derivatives can be obtained by a person skilled in the art without undue experimentation. See, for example, Burger's Medicinal Chemistry and Drug Discovery 5th ed., vol. 1st, “Principles and Practice”. Preferred pharmaceutically acceptable derivative is a salt, a solvate, an ester, a carbaminic ester and a phosphoric ester. Especially preferred pharmaceutically acceptable derivative is a salt, a solvate and an ester. Most preferred pharmaceutically acceptable derivative is a salt and an ester.

A person skilled in the art of organic chemistry knows that many organic compounds can be formed a complex with a solvent of reaction system and which can be precipitated out or crystallized out from the solvent. These complexes are widely known as a “solvate”. For example, the complex with water is known as a “hydrate”. The solvate of the compound of the present invention falls within the scope of the invention.

As used herein, the “prodrug” represent a compound which convert to the active form having a pharmacological activity by a hydrolysis in vivo (such as in a blood). The example of the pharmaceutically acceptable prodrug is described in the literature: T. Higuchi and V. Stera, Prodrugs as Novel Delivery Systems, “Bioreversible Carriers in Drug Design”, Edward B. Roche ed., American Pharmaceutical Association and Pergamon Press, A.C.S. Symposium Series, vol. 14th, (1987); and D. Freisher, S. Roman and H. Barbara, Improved oral drug delivery: solubility limitations overcome by the use of prodrugs, Advanced Drug Delivery Reviews (1996) 19(2): 115-130).

A prodrug is a carrier that releases the compound of the formula (1) which is bound covalently in vivo when administered to a patient. In general, the prodrug is prepared by the conventional method or by modifying a functional group such that the modified moiety is cleaved in vivo to give a parent compound. Examples of the prodrug include the compound wherein a hydroxy, amine or sulfhydryl group binds to an optional group such that provide a hydroxy, amine or sulfhydryl group by cleaving it when administered to a patient. Thus the representative examples of prodrug are the following ones, but not limited thereto; i.e. the derivatives with acetic ester, formic ester and benzoic ester at the functional groups such as alcohol, sulfhydryl or amine of the compound of the formula (1). In addition, when the functional group is a carboxylic acid, esters such as methyl ester, ethyl ester and double ester, and the like can be used. The esters exhibit inherently the activity in a human body and/or are hydrolyzed under in vivo condition to the active compound. The suitable pharmaceutically acceptable esters capable of hydrolyzing in vivo include those which are decomposed easily in the human body to release the parent acid compound or a salt thereof.

The compound of the present invention may be a pharmaceutically acceptable salt form thereof. The suitable salt is outlined in literature (Berge et. al., J. Pharm. Sci., 66: 1-19 (1977)).

In general, the pharmaceutically acceptable salts can be easily prepared with a desirable acid or base as needed. The resulting salts can be recovered by filtering after precipitating it out from the solution or distilling the solvent off.

The suitable additive salts may be formed with an acid forming a nontoxic salt. Examples of the salt include hydrochloride, hydrobromide, hydroiodide, sulfate, bisulfate, nitrate, phosphate, hydrogen phosphate, acetate, maleate, malate, fumarate, lactate, tartarate, citrate, formate, gluconate, succinate, pyruvate, oxalate, oxaloacetate, trifluoroacetate, saccharate, benzoate, methanesulfonate, ethanesulfonate, benzenesulfonate and p-toluenesulfonate.

Examples of the pharmaceutically acceptable salt with a base include alkaline metal salts including ammonium salt, sodium salt and potassium salt; alkaline earth metal salts including calcium salt and magnesium salt as well as salts with organic bases including a primary, secondary and tertiary amine (e.g. isopropylamine, diethylamine, ethanolamine, trimethylamine, dicyclohexylamine and N-methyl-D-glucamine).

The term “halogen” refers to fluorine, chlorine, bromine or iodine.

The term “alkyl group” or “alkyl” means a straight or branched saturated hydrocarbon chain having 1 to 10 carbon atoms and a cyclic saturated hydrocarbon chain having 3 to 10 carbon atoms. As a straight or branched hydrocarbon chain, those having 2 to 10 carbon atoms are preferred and those having 2 to 6 carbons are more preferred. More preferred examples are straight chain alkyl groups having 1 to 6 carbon atoms, especially those having 1 to 4 carbon atoms. Examples of alkyl group include methyl, ethyl, propyl, isopropyl, butyl, sec-butyl, tert-butyl, pentyl, isopentyl, neopentyl, tert-pentyl, 1-methylbutyl, 2-methylbutyl, 3-methylbutyl, hexyl and isohexyl groups, and the like.

The term “alkoxy group” or “alkoxy” means a straight or branched alkyloxy group having 1 to 10 carbon atoms and a cyclic alkyloxy group having 3 to 10 carbon atoms. As a straight or branched hydrocarbon chain, those having 2 to 10 carbon atoms are preferred and those having 2 to 6 carbons are more preferred. More preferred examples are straight chain alkoxy groups having 1 to 6 carbon atoms, especially those having 1 to 4 carbon atoms. Examples of alkoxy group include methoxy, ethoxy, propoxy, isopropoxy, butoxy, isobutoxy, tert-butoxy, pentyloxy, isopentyloxy, neopentyloxy, tert-pentyloxy, hexyloxy and isohexyloxy groups, and the like.

The term “alkylene group” or “alkylene” means a saturated hydrocarbon chain wherein a hydrogen atom is removed from each of the terminal carbons of a straight hydrocarbon chain. Preferred examples include an alkylene group having 1 to 6 carbon atoms, specifically, methylene, ethylene, trimethylene and tetramethylene groups, and the like. When an alkylene group herein used contains 1 to 3 heteroatoms selected independently from nitrogen, sulfur and oxygen atoms, the term “alkylene” includes a group of the formula: —O—(CH₂)ₘ—O—, —S—(CH₂)ₘ—S—, —NH—(CH₂)ₘ₋₁ NH—, or —O—(CH₂)ₘ—NH— (wherein m is an integer of 1 to 4), or the like.

The term “alkanoyl group” or “alkanoyl” means a straight or branched alkylcarbonyl group having 1 to 10 carbon atoms, preferably an alkylcarbonyl group having 1 to 6 carbon atoms, more preferably an alkylcarbonyl group having 1 to 4 carbon atoms. Examples of alkanoyl group include acetyl, propionyl, butyryl, valeryl and pivaloyl groups, and the like.

The term “alkenyl group” or “alkenyl” means a straight or branched hydrocarbon chain having 2 to 10 carbon atoms and containing at least one double bond, preferably an alkenyl group having 2 to 6 carbon atoms, more preferably an alkenyl group having 2 to 4 carbon atoms Examples of alkenyl group include vinyl, 1-propenyl, allyl, isopropenyl, butenyl, butadienyl and pentenyl groups, and the like.

As herein used throughout the claims and specification, when the term “mono- or di-alkyl” refers to di-alkyl, the alkyl moieties may be the same or independent from each other.

The cycloalkyl or cycloalkyl group as used herein is C3-10 cyclic hydrocarbon group and includes for example, cyclopropyl, cyclobutyl, cyclopentyl, cyclohexyl, cycloheptyl, cyclooctyl, cyclononyl groups, and the like and preferably C3-6 cyclic hydrocarbon group.

The cycloalkoxy and cycloalkoxy group as used herein is oxy group substituted by C3-10 cyclic hydrocarbon and includes, for example cyclopropyloxy, cyclobutyloxy, cyclopentyloxy, cyclohexyloxy, and the like and preferably oxy group substituted by C3-6 cyclic hydrocarbon group.

The heterocycle or heterocyclic group as used herein includes 5 to 8 membered heterocyclic group including 1 to 4 heteroatoms selected from oxygen, sulfur and nitrogen atoms and bicyclic or tricyclic fused heterocyclic group fused thereto. Specific examples of the heterocyclic group include, for example, a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl or tetrahydroquinolyl group, and the like.

The homocycle or homocyclic group as used herein include, for example, 3 to 7 membered carbocyclic group optionally fused, such as C6-10 aryl group (e.g. phenyl and naphthyl group, and the like), C3-10 cycloalkyl group (e.g. cyclopropyl, cyclobutyl, cyclopentyl, cyclohexyl, cycloheptyl, and the like), C3-10 cycloalkenyl group (e.g., cyclopropenyl, cyclobutenyl, cyclopentenyl, cyclohexenyl, cycloheptenyl, and the like).

### EFFECT OF THE INVENTION

The compound (1) of the present invention has an inhibitory activity against CETP and shows the effects on increasing HDL cholesterol level and decreasing LDL cholesterol. Thus the compound is useful in a prophylaxis and a treatment of diseases such as arteriosclerosis and hyperlipidemia, and the like.

The present compound (1) can be administered either orally or parenterally, and can be formulated into a suitable pharmaceutical preparations with a conventional pharmaceutically acceptable carriers used therefor.

The pharmaceutically acceptable salts of the compound (1) include, for example, alkali metal salts such as lithium, sodium or potassium salt; alkali earth metal salts such as calcium or magnesium salt; salts with zinc or aluminum; salts with organic bases such as ammonium, choline, diethanolamine, lysine, ethylenediamine, tert-butylamine, tert-octylamine, tris(hydroxymethyl)aminomethane, N-methylglucosamine, triethanolamine or dehydroabiethylamine; salts with inorganic acids such as hydrochloric acid, hydrobromic acid, hydroiodic acid, sulfuric acid, nitric acid or phosphoric acid; salts with organic acids such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, benzenesulfonic acid or toluenesulfonic acid; or salts derived from acidic amino acids such as aspartic acid or glutamic acid.

Additionally, the pharmaceutically acceptable salts of the compound of the formula (1) include, for example, quaternary salts formed between a compound of the formula (1) and an alkyl halide or a phenylalkyl halide.

Preferred pharmaceutical preparations for oral administration of the present compound include solid formulations such as tablets, granules, capsules or powders; and liquid formulations such as solutions, suspensions or emulsions. Preferred pharmaceutical preparations for parenteral administration include injections or infusions formulated with injectable distilled-water, physiological saline or aqueous glucose solution; suppository; or inhalation preparation, and the like.

These pharmaceutical preparations comprise a compound (1) of the present invention or a pharmaceutically acceptable salt thereof and a pharmaceutically acceptable carrier which is usually used for oral or parenteral administration. The pharmaceutically acceptable carriers for oral administration include, for example, a binder (syrup, gum acacia, gelatin, sorbit, tragacanth, polyvinylpyrrolidone, and the like), an excipient (lactose, sugar, cornstarch, potassium phosphate, sorbit, glycine, and the like), a lubricant (magnesium stearate, talc, polyethylene glycol, silica, and the like), a disintegrant (potato starch, and the like), and a wetting agent (anhydrous sodium lauryl sulfate, and the like).

Also the pharmaceutically acceptable carriers for parenteral administration include, for example, injectable distilled-water, physiological saline and aqueous glucose solution.

The dose of a compound (1) of the present invention or a pharmaceutically acceptable salt thereof varies depending on the administration route, age, body weight, disease, and condition/severity, of the patient. It however can usually be in the range of about 0.001-1,000 mg/kg/day, preferably in the range of about 0.01-100 mg/kg/day, more preferably in the range of about 0.1-10 mg/kg/day.

The compounds (1) of the present invention have an inhibitory activity against CETP and show an activity of increasing HDL cholesterol level and an activity of lowering LDL cholesterol level. Accordingly, they are useful in the prophylaxis or treatment of a subject (particularly, mammal including human) suffering from arteriosclerosis such as atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular diseases, angina, ischemia, cardiac ischemia, stroke, myocardial infarction, reperfusion injury, restenosis after angioplasty, hypertension, cerebral infarction, cerebral stroke, diabetes, vascular complication of diabetes, thrombotic diseases, obesity, endotoxemia, metabolic syndrome, cerebrovascular disease, coronary artery disease, ventricular dysfunction, cardiac arrhythmia, pulmonary vascular disease, reno-vascular disease, renal disease, splanchnic vascular disease, vascular hemostatic disease, fatty liver disease, steatohepatitis, inflammatory disease, autoimmune disorders and other systemic disease indications, immune function modulation, pulmonary disease, anti-oxidant disease, sexual dysfunction, cognitive dysfunction, schistosomiasis, cancer, regression of xanthoma, Alzheimer's disease, or the like.

In addition, the compounds of the present invention may be used in combination with other drugs useful for treatment of these diseases. For example, a compound of the present invention may be used in combination with an inhibitor of cholesterol synthesis such as HMG-CoA reductase inhibitor; an inhibitor of cholesterol absorption such as anion exchange resin; a triglyceride lowering agent such as fibrates, niacin and fish oil; an antihypertensive such as ACE inhibitor, angiotensin receptor blocker, calcium antagonist and beta blocker; an antiobesity agent such as central anorectic, lipase inhibitor and CB1 antagonist; an antidiabetic agent such as insulin sensitizer, D2 agonist, sulfonylurea, biguanide, α-glucosidase inhibitor, SGLT inhibitor and BPPIV inhibitor; or other cholesterol reducer such as ACAT inhibitor.

The compound (1) of the present invention may be prepared by the following methods, which should not be construed to be limited thereto.

In each process for preparing a compound of the formula (1) described above, when protection of a functional group contained in any compound is needed, the protection can be carried out in a conventional manner ad libitum. General statement related to protecting groups and their use is provided by Greene, Protective Groups in Organic Synthesis, John Wiley and Sons, New York, 1991.

Further in each process, the reaction can be carried out by the conventional method and a procedure for isolation and the purification may be selected from the conventional method such as crystallization, recrystallization and chromatography and preparative HPLC, and the like as appropriate, or may be combined with one another.

### [Method I]

The compound (1) can be prepared by the following method I.

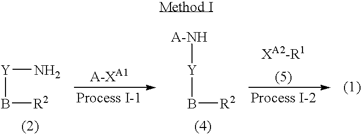

wherein the X^{A1} and X^{A2} are a leaving group and the other symbols have the same meanings as defined above

### (Process I-1)

The compound (4) can be prepared by reacting the compound (2) with the compound (3) in a solvent in the presence of a base.

Any solvent which dose not disturb the reaction can be preferably used as a solvent used, and include, for example, ethers including diethylether, tetrahydrofuran (THF), dioxane, 1,2-methoxyethane, diglyme; hydrocarbons including benzene, toluene, hexane, xylene; alcohols including methanol, ethanol, isopropyl alcohol, tert-butanol; esters including ethyl acetate, methyl acetate, butyl acetate; polar solvents including acetone, N,N-dimethylformamide, dimethyl sulfoxide, which can be used alone or in a combination thereof. Preferred solvents in this reaction include ethanol, dioxane, toluene and N,N-dimethylformamide.

A conventional base may be used as a base, and includes for example, alkaline metal hydride including sodium hydride and potassium hydride; alkaline metal alkoxide including sodium ethoxide, sodium methoxide, sodium tert-butoxide and potassium tert-butoxide; alkyl lithium including n-butyl lithium and sec-butyl lithium; alkaline metal amide including lithium diisopropylamide, sodium amide and lithium bis(trimethylsilyl)amide; alkaline metal carbonate including sodium carbonate, potassium carbonate, sodium bicarbonate and potassium bicarbonate; alkaline metal hydroxide including lithium hydroxide, sodium hydroxide and potassium hydroxide; alkaline metal phosphate including sodium phosphate and potassium phosphate; organic base including triethylamine, diisopropylethylamine, pyridine and N-methylmorpholine; preferably triethylamine, sodium bicarbonate, sodium tert-butoxide, diisopropylethylamine, sodium hydride and potassium tert-butoxide.

The leaving group includes a halogen atom including chlorine atom, bromine atom, iodine atom, and a substituted sulfonyloxy group including methanesulfonyloxy group, p-toluenesulfonyloxy group and trifluoro-methanesulfonyloxy group.

### (Process I-2)

The compound (1) can be prepared by reacting the compound (4) with the compound (5) in a same manner as in process I-1 or I′-1 described below.

### (Process I′-1)

The process (I-1) may be replaced by the following process I′-1.

In the above process I-1, the compound (4) can also be prepared by reacting the compound (2) with the compound (3) in the presence or absence of a base or in the presence of a metal catalyst such as palladium catalyst in a suitable solvent.

A conventional palladium catalyst can be used as a palladium catalyst, and include palladium acetate, tetrakis(triphenylphosphine)-palladium, tris(dibenzylideneacetone)dipalladium, dichlorobis(triphenyl-phosphine)palladium, dichlorobis(tri-o-tolylphosphine)palladium, bis-(triphenylphosphine)palladium acetate, or the like.

As the base, alkaline metal hydroxide including sodium hydroxide, potassium hydroxide; alkaline earth metal hydroxide including barium hydroxide; alkaline metal alkoxide including sodium methoxide, sodium ethoxide, potassium ethoxide, sodium tert-butoxide, potassium tert-butoxide; alkaline metal carbonate including sodium carbonate, potassium carbonate, cesium carbonate; alkaline metal bicarbonate including sodium bicarbonate, potassium bicarbonate; alkaline metal phosphate including potassium phosphate; amines including triethylamine, diisopropyl-ethylamine, methylpiperidine, dicyclohexylmethylamine; and pyridines including pyridine, 4-dimethylaminopyridine can be preferably used.

Additionally, phosphines may be added in the present reaction. As the phosphines, triphenylphosphine, tributylphosphine, tri-tert-butylphosphonium tetrafluoroborate, 1,3-bis(diphenylphosphino)propane, 2,2′-bis(diphenylphosphino)-1,1′-binaphthyl, 1,1′-bis(diphenylphosphino)-ferrocene, 2-(di-tert-butylphosphino)biphenyl, 2-dicyclohexylphosphino-2′-(N,N-dimethylamino)biphenyl, 2-(dicyclohexylphosphino)biphenyl, and the like can be preferably used.

Any solvent which dose not disturb the reaction can be preferably used as a solvent used in the reaction, and include, for example, ethers including diethylether, tetrahydrofuran (THF), dioxane, 1,2-methoxyethane, diglyme; hydrocarbons including benzene, toluene, hexane, xylene; alcohols including methanol, ethanol, isopropyl alcohol, tert-butanol; esters including ethyl acetate, methyl acetate, butyl acetate; polar solvents including acetone, N,N-dimethylformamide, dimethyl sulfoxide, and which can be used alone or in a combination thereof.

### (Process I′-2)

The compound (1) can be prepared by reacting the compound (4) with the compound (5) in a same manner as in process I′-1 or I-1.

### [Method II]

The compound can be prepared by a method II.

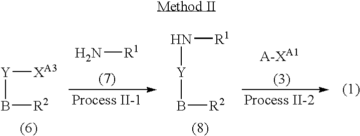

wherein the X^{A3} is a leaving group and the other symbols have the same meanings as defined above

### (Process II-1)

The compound (8) can be prepared by reacting the compound (6) with the compound (7) in a same manner as process I-1.

### (Process II-2)

The compound (1) can be prepared by reacting the compound (8) with the compound (3) in a same manner as in process I-1 or I′-1.

### [Method III]

The compound (1) can be prepared by a method III.

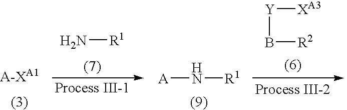

wherein the symbols have the same meanings as defined above

### (Process III-1)

The compound (9) can be prepared by reacting the compound (3) with the compound (7) in a same manner as in process I-1 or process I′-1.

### (Process III-2)

The compound (1) can be prepared by reacting the compound (9) with the compound (6) in a same manner as in process I-1.

### [Method IV]

The compound of the general formula (1-a):

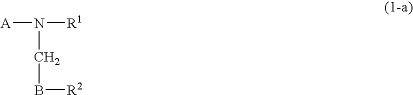

wherein the symbols have the same meanings as defined above, that is, those wherein Y is a methylene group in the compound of the formula (1), can be prepared by a following method IV.

The compound (1-a) can be prepared by reducing the compound of a general formula (6′):

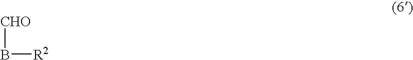

wherein the symbols have the same meanings as defined above, or its corresponding carboxylic acid or its corresponding carboxylic acid ester to provide the compound of a general formula (6″):

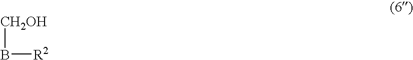

wherein the symbols have the same meanings as defined above, followed by halogenating the resulting compound and then reacting the resulting compound with the above compound (9) in a solvent in the presence of a base.

The reduction can be carried out by treating a starting compound with a reducing agent in a suitable solvent. Boron hydrides (sodium borohydride, and the like) and aluminum hydrides (lithium aluminum hydride, diisobutylaluminum hydride, and the like) can be preferably used as a reducing agent.

The halogenation can be carried out by treating a starting compound with a halogenating agent in a suitable solvent. As the halogenating agent, a conventional halogenating agent including thionyl chloride, phosphorus oxychloride, as well as carbon tetrahalide (e.g., carbon tetrachloride, carbon tetrabromide, and the like) and phosphines (e.g., triphenylphosphine, tritolylphosphine, triethylphosphine, and the like) can be preferably used.

A conventional base may be used as a base and include for example, alkaline metal hydride including sodium hydride and potassium hydride; alkaline metal alkoxide including sodium ethoxide, sodium methoxide, sodium tert-butoxide and potassium tert-butoxide; alkyl lithium including n-butyl lithium and sec-butyl lithium; alkaline metal amide including lithium diisopropylamide, sodium amide and lithium bis(trimethylsilyl)amide; alkaline metal carbonate including sodium carbonate, potassium carbonate, sodium bicarbonate and potassium bicarbonate; alkaline metal hydroxide including lithium hydroxide, sodium hydroxide and potassium hydroxide; alkaline metal phosphate including sodium phosphate and potassium phosphate; organic base including triethylamine, diisopropylethylamine, pyridine and N-methylmorpholine; preferably triethylamine, sodium bicarbonate, sodium tert-butoxide, diisopropylethylamine, sodium hydride and potassium tert-butoxide.

Any solvent which dose not disturb the reaction can be preferably used as a solvent used, and include, for example, ethers including diethylether, tetrahydrofuran (THF), dioxane, 1,2-methoxyethane, diglyme; hydrocarbons including benzene, toluene, hexane, xylene; alcohols including methanol, ethanol, isopropyl alcohol, tert-butanol; esters including ethyl acetate, methyl acetate, butyl acetate; polar solvents including acetone, N,N-dimethylformamide, dimethyl sulfoxide, which can be used alone or in a combination thereof. Preferred solvents in this reaction include ethanol, dioxane, toluene and N,N-dimethylformamide.

### [Method IV′]

The compound (1-a) can also be prepared by the following method IV′.

The compound (1-a) can also be prepared by halogenating the above compound (6″) according to the above method IV, followed by reacting the resulting compound with the above compound (7) in a solvent in the presence of a base to provide the compound of a general formula (8′)

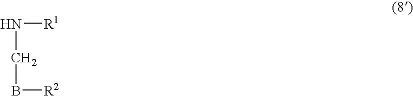

wherein the symbols have the same meanings as defined above, then reacting the resulting compound with the compound (3) according to the above process II-2.

### [Method IV″]

In the above method IV′, the compound (8′) can be prepared by reacting the compound (6′) with the compound (7) in a solvent in the presence of a reducing agent.

Any solvent which dose not disturb the reaction can be preferably used as a solvent used in the reaction, and include for example, halogenated solvents including 1,2-dichloroethane, dichloromethane, chloroform, ethers including diethylether, tetrahydrofuran (THF), dioxane, 1,2-methoxyethane, diglyme; hydrocarbons including benzene, toluene, hexane, xylene; alcohols including methanol, ethanol, isopropyl alcohol, tert-butanol; esters including ethyl acetate, methyl acetate, butyl acetate; polar solvents including acetone, N,N-dimethylformamide, dimethyl sulfoxide, and which can be used alone or in a combination thereof. Especially preferred solvent in this reaction includes 1,2-dichloroethane, dichloromethane and toluene. The reducing reagent includes sodium borohydrides including sodium triacetoxyborohydride, sodium cyanoborohydride; and aluminum hydrides including lithium aluminium hydride and diisobutylaluminium hydride.

### [Method V]

The compound of a general formula (1-b):

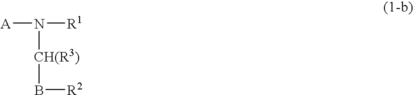

wherein R³ is an alkyl group and the other symbols have the same meanings as defined above, those wherein Y is an alkyl group in the compound (1), can be prepared by undergoing a conventional Grignard reaction of the compound (6′) with a reagent of a general formula: R³MgBr wherein the symbol has the same meaning as defined above, to provide the compound of a general formula (6′″):

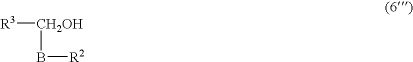

wherein the symbols have the same meanings as defined above, followed by halogenating the resulting compound in a same manner as in the above method IV, then reacting the resulting compound with the above compound (9) in a solvent in the presence of a base.

### [Method V′]

The compound (1-b) can also be prepared by the following method V′.

The compound (1-b) can also be prepared by halogenating the above compound (6′″) according to the above method V, followed by reacting the resulting compound with the above compound (7) in a solvent in the presence of a base, to provide a compound of a general formula (8″):

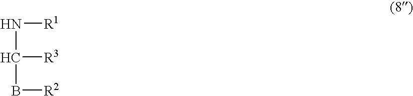

wherein the symbols have the same meanings as defined above, then reacting the resulting compound with the compound (3) according to the above process II-2.

### [Method VI]

The compound of a general formula (1-c):

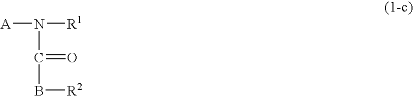

wherein the symbols have the same meanings as defined above, those wherein Y is an methylene group substituted by an oxo group in the compound (1), can be prepared by oxidizing the compound (6′) to provide the compound of a general formula (6″″):

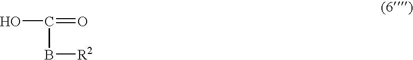

wherein the symbols have the same meanings as defined above, followed by halogenating the resulting compound according to the above method IV, then reacting the resulting compound with the above compound (9) in a solvent in the presence of a base.

### [Method VI′]

The compound (1-c) can also be prepared by the following method VI′.

The compound (1-c) can be prepared by halogenating the above compound (6″″) according to the above method VI, followed by reacting the resulting compound with the above compound (7) in a solvent in the presence of a base, to provide a compound of a general formula (8′″):

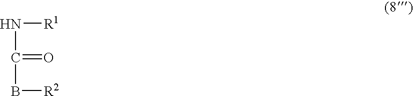

wherein the symbols have the same meanings as defined above, then reacting the resulting compound with the compound (3) according to the above process II-2.

### [Method VII]

The compound (1-a) can be prepared by the following method VII.

The compound (1-a) can also be prepared by reducing the compound of a general formula (6′″″):

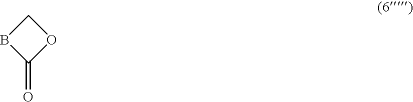

wherein the symbols have the same meanings as defined above, to provide the compound of a general formula (6″″″):

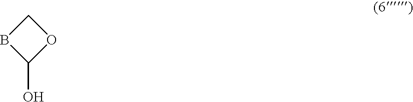

wherein the symbols have the same meanings as defined above, then reacting the resulting compound with the above compound (9) in a same manner as in the method IV″, to provide the compound of a general formula (1-d):

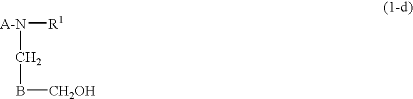

wherein the symbols have the same meanings as defined above, and interconverting on R² moiety.

### [Method VII′]

The compound (1-a) can also be prepared by reacting the above compound (6″″″) with the above compound (7), in a same manner as in the above method IV″ to provide the compound of a general formula (8″):

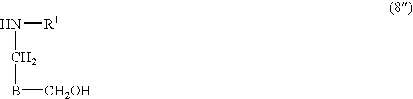

wherein the symbols have the same meanings as defined above, followed by reacting the resulting compound with the compound (3) according to the above process II-2 to provide the compound (1-d), then interconverting on R² moiety.

In addition, substituents on the A, B, R¹ and R² may be further interconverted according to the known method after or before a synthesis of the compound (1).

A² can be interconverted by the following methods (EA) to (EL).

In the following each process, unless otherwise specified, a conventional base can be used as the base, and for example, alkaline metal hydride including sodium hydride, potassium hydride; alkaline metal hydroxide including sodium hydroxide, potassium hydroxide; alkaline earth metal hydroxide including barium hydroxide; alkaline metal alkoxide including sodium methoxide, sodium ethoxide, potassium ethoxide, potassium tert-butoxide; alkaline metal carbonate including sodium carbonate, potassium carbonate, cesium carbonate; alkaline metal bicarbonate including sodium bicarbonate, potassium bicarbonate; amines including triethylamine, diisopropylethylamine, methylpiperidine, dimethylaniline, 1,8-diazabicyclo[5.4.0]undecene, 1,4-diazabicyclo[2.2.2]octane, 1,5-diazabicyclo[4.3.0]nonene; pyridines including pyridine, dimethylaminopyridine can be preferably used.

Also in the following each process, a conventional acid can be used as the acid, and for example, unless otherwise specified, an inorganic acid (e.g. hydrochloric acid, nitric acid and sulfuric acid); organic acid represented by sulfonic acids (e.g. methanesulfonic acid, p-toluenesulfonic acid and trifluoromethanesulfonic acid, and the like) can be preferably used.

Also in the following each process, any solvent which dose not disturb the reaction can be used as the solvent and specifically include hydrocarbons including pentane, hexane; aromatic hydrocarbons including benzene, toluene, nitrobenzene; halogenated hydrocarbons including dichloromethane, chloroform; ethers including diethylether, tetrahydrofuran; amides including dimethylformamide, N-methylpyrrolidone, 1,3-dimethylimidazolidin-2-one; sulfoxides including dimethylsulfoxide; alcohols including methanol, ethanol; esters including ethyl acetate, butyl acetate; ketones including acetone, methyl ethyl ketone; nitrites including acetonitrile; water, or a mixed solvent thereof.

Also in the following each process, the leaving group includes a halogen atom including chlorine atom, bromine atom, iodine atom, and a substituted sulfonyloxy group including methanesulfonyloxy group, trifluoromethanesulfonyloxy group and toluenesulfonyloxy group.

(EA) The compound wherein A¹ is a tetrazolyl group and A² is an alkyl group or a substituted alkyl group can be prepared by alkylating a compound wherein A¹ is a tetrazolyl group and A² is a hydrogen atom.

The alkylation can be carried out by reacting with a compound of the formula:

A^{2A}-Z²

wherein A^{2A} is an alkyl group or a substituted alkyl group and Z² is a leaving group, in a suitable solvent in the presence or absence of a base, or by reacting with a compound of the formula:

A^{2A}-OH

wherein the symbol has the same meaning as defined above, in a suitable solvent in the presence of phosphines and azodicarboxylic esters.

N-alkylation of a compound wherein A¹ is a nitrogen-containing heterocyclic group can be carried out in a similar manner as above.

The reaction proceeds more preferably when a catalytic amount of an alkaline metal iodide (e.g., potassium iodide, and the like) is added.

Both phosphines and azodicarboxylic esters which usually employed in Mitsunobu reaction can be preferably used. Phosphines include, for example, triphenylphosphine, tributylphosphine, and the like, and azodicarboxylic esters include diethyl azodicarboxylate, diisopropyl azodiformate, and the like.

(EB) The compound wherein A¹ is 2-oxodihydropyrimidinyl group and A² is an alkyl group or a substituted alkyl group can be prepared by alkylating a compound of the formula (1) wherein A¹ is 2-hydroxypyrimidinyl group and A² is a hydrogen atom, with a compound of the formula:

A^{2A}-Z²

wherein the symbols have the same meaning as defined above.

The reaction can be carried out in the same manner as in the above (EA).

(EC) The compound wherein A² is an optionally substituted amino group or a group of the formula:

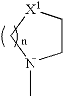

wherein the symbols have the same meaning as defined above, can be prepared by reacting a compound of the formula (1) wherein A² is a halogen atom, with a corresponding amine or a compound of the formula:

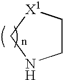

wherein the symbols have the same meanings as defined above.

The reaction can be carried out in the presence or absence of a base, and in the or absence of a palladium catalyst in a suitable solvent.

As the palladium catalyst, a conventional palladium catalyst including palladium acetate, tetrakis(triphenylphosphine)palladium, tris(dibenzylideneacetone)dipalladium, dichlorobis(triphenylphosphine)-palladium, dichlorobis(tri-o-tolylphosphine)palladium, bis-(triphenylphosphine)palladium acetate, or the like can be used.

As the base, alkaline metal hydroxide including sodium hydroxide, potassium hydroxide; alkaline earth metal hydroxide including barium hydroxide; alkaline metal alkoxide including sodium methoxide, sodium ethoxide, potassium ethoxide, potassium tert-butoxide; alkaline metal carbonate including sodium carbonate, potassium carbonate, cesium carbonate; alkaline metal bicarbonate including sodium bicarbonate, potassium bicarbonate; alkaline metal phosphate including potassium phosphate; amines including triethylamine, diisopropylethylamine, methylpiperidine, dicyclohexylmethylamine; and pyridines including pyridine, 4-dimethylaminopyridine can be preferably used.

Additionally, phosphines may be added in the present reaction. As the phosphines, triphenylphosphine, tributylphosphine, tri-tert-butylphosphonium tetrafluoroborate, 1,3-bis(diphenylphosphino)propane, 2,2′-bis(diphenylphosphino)-1,1′-binaphthyl, 1,1′-bis(diphenylphosphino)-ferrocene, 2-(di-tert-butylphosphino)biphenyl, 2-dicyclohexylphosphino-2′-(N,N-dimethylamino)biphenyl, 2-(dicyclohexylphosphino)biphenyl, and the like can be preferably used as the phosphines.

(ED) The compound wherein A² is an optionally substituted amino group can be prepared by coupling a compound of the formula (1) wherein A² is a halogen atom with a compound of the formula:

(R²⁰)₃Sn—NR²¹R²²

wherein R²⁰ is an alkyl group and NR²¹R²² is an optionally substituted amino group.

The coupling reaction can be carried out in the presence of a palladium catalyst in the presence or absence of a base in a suitable solvent.

The palladium catalysts, bases, and phosphines described in the above (EC) can be used in the same manner as in the above (EC).

(EE) The compound wherein A² is a cyano group can be prepared by cyanating a compound of the formula (1) wherein A² is a halogen atom.

The cyanation can be carried out by reacting a starting compound with a metal cyanide including sodium cyanide, potassium cyanide, or zinc cyanide in the presence of a palladium catalyst in a suitable solvent.

The same palladium catalyst as that described in the above (EC) can be preferably used.

(EF) The compound wherein A² is an optionally substituted alkoxycarbonyl group can be prepared by reacting a compound of the formula (1) wherein A² is a halogen atom, with a corresponding alkylalcohol under carbon monoxide atmosphere using a palladium catalyst in the presence of a base in a suitable solvent.

The same palladium catalyst and base as those described in the above (EC) can be preferably used.

Additionally, the reaction can be more preferably carried out by adding a ligand, and phosphines described in the above (EC) can be preferably used as the ligand.

(EG) The compound wherein A² is an optionally substituted alkenyl group can be prepared by coupling a compound of the formula (1) wherein A² is a halogen atom with a corresponding alkene.

The coupling reaction can be carried out in the presence of a palladium catalyst in the presence or absence of a base in a suitable solvent.

The same palladium catalyst as that described in the above (EC) can be preferably used.

The same base as that described in the above (EC) can be preferably used and silver carbonate can also be used.

Additionally, the reaction can be more preferably carried out by adding a ligand, and phosphines described in the above (EC) can be preferably used as the ligand.

(EG′) The compound wherein A² is an optionally substituted alkenyl group can be prepared by dehydration reaction of a compound having a hydroxyalkyl group in a substituent A². The dehydration reaction can be carried out by treating a starting compound with acid.

As the acid, a conventional acid can be used. For example, an inorganic acid (e.g. hydrochloric acid, nitric acid and sulfuric acid) or an organic acid (e.g. methane sulfonic acid, p-toluenesulfonic acid, acetic acid, trifluoroacetic acid, trifluoromethanesulfonic acid, and the like) can be preferably used.

(EH) The compound wherein A² is a boronic acid ester can be prepared by reacting with the compound wherein A² is a leaving group with a trialkoxyborane (trimethoxyborane, triisopropoxyborane, and the like), a dialkoxyborane (pinacolborane, and the like) or a tetraalkoxydiboron (bis(pinacolato)diboron, and the like) in the presence of palladium catalyst. The leaving group includes a halogen atom including chlorine atom, bromine atom, iodine atom, and a substituted sulfonyloxy group including methanesulfonyloxy group, trifluoromethanesulfonyloxy group, and toluenesulfonyloxy group. The reaction can be carried out in the same manner as in the above (EC).

(EH′) The compound wherein A² is a hydroxy group can be prepared by reacting the compound wherein A² is a boronic acid ester with the peroxide. Aqueous hydrogen peroxide, m-chloroperbenzoic acid and OXONE™ (DuPont Co. Ltd), and the like can be preferably used as the peroxide.

(EI) The compound wherein A² is an alkoxy group or a substituted alkoxy group can be prepared by alkoxylating the compound (1) wherein A² is a halogen atom.

The alkoxylation can be carried out by optionally adding a copper catalyst to react with a corresponding alcohol in a suitable solvent or solvent-free in the presence of a base.

The same base as described in the above (EC), in particular, cesium carbonate can be preferably used.

The copper catalyst including copper iodide, copper bromide, copper chloride, copper acetate, copper trifluoromethanesulfonate, and the like can be preferably used.

Additionally, this reaction proceeds more preferably when 1,10-phenanthroline, 2-aminopyridine, or the like is added.

(EJ) The compound wherein A² is an optionally substituted alkyl group, an optionally substituted heterocyclic group or an optionally substituted aryl group can be prepared by coupling a compound of the formula (1) wherein A² is a leaving group with a corresponding alkyl, aryl or heterocyclic boronic acid or a corresponding alkyl, aryl or heterocyclic boronic acid ester.

The coupling can be carried out in the presence of a palladium catalyst, and in the presence or absence of a base in a suitable solvent.

This reaction can be carried out in the same manner as in the above (EC).

The leaving group is the same as defined above (EH).

(EJ′) The compound wherein A² is an optionally substituted alkyl group, an optionally substituted heterocyclic group or an optionally substituted aryl group can be prepared by coupling a compound of the formula (1) wherein A² is a boronic acid or a boronic acid ester with an alkyl, an aryl or a heterocyclic group which have a leaving group.

The coupling can be carried out in the presence of a palladium catalyst, and in the presence or absence of a base in a suitable solvent. This reaction can be carried out in the same manner as in the above (EC). The leaving group is the same as defined above (EH).

(EK) The compound wherein A² is an alkoxycarbonylalkylsulfonyl group can be prepared by reacting a compound of the formula (1) wherein A² is a halogen atom with an alkoxycarbonylalkylsulfinic acid alkaline metal salt.

The alkoxycarbonylalkylsulfinic acid alkaline metal salt can be prepared according to the method described, for example, in Baskin et. al., Tetrahedron Lett., 43, 8479 (2002).

Additionally, this reaction can be carried out in the presence of a copper catalyst in a suitable solvent according to the method described in the said literature.

The same copper catalyst as described in (EI) above can be used, and in particular, copper iodide can be preferably used.

(EL) The compound wherein A² is a group of the formula:

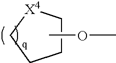

wherein the symbols have the same meaning as defined above, can be prepared by condensing a compound wherein A² is a hydroxy group with a compound of the formula:

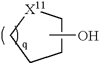

wherein X¹¹ is O, SO, SO₂ or NR^{p} (R^{p} is a protecting group) and q is an integer from 1 to 4, and as needed, removing the protecting group for amino group.

As a protecting group, a conventional protecting group including benzyloxycarbonyl group, tert-butoxycarbonyl group, and the like can be used.

The reaction can be carried out in a suitable solvent in the presence of phosphines and azodicarboxylic esters. The reaction can be carried out in the same manner as in the above (EA).

The removal of a protecting group can be carried out in a conventional manner including catalytic reduction, acid-treatment, and the like, depending on the type of a protecting group.

The same manner as in the above reactions (EA) to (EL) for conversions of A² can also be applied for conversion of the other substituent as needed.

Additionally, a substituent(s) of a compound (1) of the present invention can be converted into different one(s) within the scope of the compound (1) according to the following methods as appropriate.

In the following each process, a conventional base can be used as the base, and unless otherwise specified, the base described in the above (EA) can be preferably used.

Additionally, in the following each process, a conventional acid can be used as the acid, and unless otherwise specified, a mineral acid including hydrochloric acid, nitric acid, sulfuric acid, or an organic acid represented by sulfonic acids (e.g., methanesulfonic acid, p-toluenesulfonic acid, trifluoromethanesulfonic acid) or carboxylic acids (e.g., acetic acid, trifluoroacetic acid) can be preferably used.

Further additionally, in the following each process, any solvent which dose not disturb the reaction can be used, and as such, the solvent described in the above (EA) can be preferably used.

The leaving group includes a halogen atom including chlorine atom, bromine atom, iodine atom, and a substituted sulfonyloxy group including methanesulfonyloxy group, trifluoromethanesulfonyloxy group, and toluenesulfonyloxy group.

(E1) The compound wherein A is a heterocyclic group substituted by an optionally substituted amino group or a group of the formula:

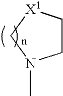

wherein the symbols have the same meanings as defined above, can be prepared by reacting a compound (1) wherein A is a heterocyclic group substituted by an optionally substituted alkylsulfonyloxy group, with a corresponding amine or a compound of the formula:

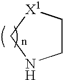

wherein the symbols have the same meaning as defined above in the presence of a palladium catalyst, and in the presence or absence of a base in a suitable solvent or solvent-free.

The reaction can be carried out in the same manner as in the above (EC).

(E2) The compound wherein A is a heterocyclic group substituted by an optionally substituted amino group or a group of the formula:

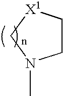

wherein the symbols have the same meanings as defined above, can also be prepared by reacting a compound wherein A is a heterocyclic group substituted by a halogen atom or an optionally substituted alkylsulfonyloxy group, with a corresponding amine or a compound of the formula:

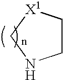

wherein the symbols have the same meanings as defined above.

The reaction can be carried out by optionally adding a copper catalyst in the presence or absence of a base in a suitable solvent. Copper iodide, copper bromide, copper chloride, copper acetate, copper trifluoromethanesulfonate, and the like can be preferably used as the copper catalyst.

The same base as that described in the above (EC) can be preferably used.

Additionally, the reaction proceeds more preferably when N,N′-dimethylethylenediamine, 1,10-phenanthroline, ethylene glycol, phenylphenol, and the like is added.

(E3) The compound wherein A is a heterocyclic group substituted by an optionally substituted alkylsulfanyl group can be prepared by reacting a compound wherein A is a heterocyclic group substituted by a halogen atom or an optionally substituted alkylsulfonyloxy group with a corresponding alkylthiol.

The reaction can be carried out in the same manner as in that described in the above (EI) and more preferably facilitated by adding 1,10-phenanthroline or ethylene glycol.

(E4) The compound wherein A is a heterocyclic group substituted by an optionally substituted heterocyclic group can be prepared by coupling a compound wherein A is a heterocyclic group substituted by a halogen atom or an optionally substituted alkylsulfonyloxy group with a corresponding heterocyclic tin compound or a corresponding heterocyclic boron compound.

The reaction can be carried out in the same manner as in the above (ED) or (EI).

(E5) The compound wherein A is a heterocyclic group substituted by an alkoxy group can be prepared by reacting a compound wherein A is a heterocyclic group substituted by a halogen atom or an alkylsulfonyl group with a corresponding alkaline metal alkoxide in a suitable solvent. The corresponding alkaline metal alkoxide can be obtained by treating a corresponding alkylalcohol with alkaline metal hydride or alkaline metal in the said solvent.

(E6) The compound having an aminoalkyl group as a substituent on A can be prepared by catalytically reducing a compound having a cyano group or a cyanoalkyl group as a substituent on A.

The catalytic reduction can be carried out by using a catalyst under hydrogen atmosphere in a suitable solvent in a conventional manner. The catalyst includes a palladium catalyst including palladium-carbon, a nickel catalyst including Raney nickel, a platinum catalyst including platinum-carbon, and the like.

(E7) The compound having an optionally substituted mono- or di-alkylsulfamoylaminoalkyl group as a substituent on A can be prepared by reacting a compound having an aminoalkyl group as a substituent on A with a corresponding halogenated mono- or di-alkylsulfamoyl.

The reaction can be carried out in a suitable solvent in the presence of a base.

(E8) The compound having an optionally substituted mono-alkylcarbamoylaminoalkyl group as a substituent on A can be prepared by reacting a compound having an aminoalkyl group as a substituent on A with a corresponding alkyl isocyanate in a suitable solvent.

(E9) The compound having a group of the formula:

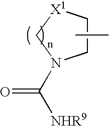

wherein R⁹ is an alkyl group and the other symbols have the same meanings as defined above as a substituent on A can be prepared by reacting a compound having a group of the formula:

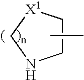

wherein the symbols have the same meanings as defined above as a substituent on A with a corresponding alkyl isocyanate (R⁹NCO). The reaction can be carried out in the same manner as in the above (E8).

(E10) The compound having an optionally substituted mono- or di-alkylcarbamoylaminoalkyl group as a substituent on A can be prepared by condensing a compound having an aminoalkyl group as a substituent on A with an optionally substituted mono- or di-alkylamine using a carbonylating agent in a suitable solvent in the presence or absence of a base.

A conventional carbonylating agent such as carbonyldiimidazole, phosgene, triphosgene, and the like can be used.

(E11) The compound having a morpholinylcarbonylamino group as a substituent on A can be prepared by condensing a compound having an amino group as a substituent on A with morpholine using a carbonylating agent in a suitable solvent. The reaction can be carried out in the same manner as in the above (E10).

(E12) The compound having a group of the formula:

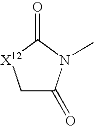

wherein X¹² is O or NH, as a substituent on A can be prepared by treating a compound having a group of the formula:

H—X¹²—CH₂—CONH—

wherein the symbols have the same meanings as defined above as a substituent on A with a carbonylating agent in a suitable solvent.

The reaction can be carried out in the same manner as in the above

(E10).

(E12) The compound having a group of the formula:

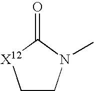

wherein X¹² is O or NH, as a substituent on A can be prepared by treating a compound having a group of the formula:

H—X¹²—CH₂—CH₂—NH—

wherein the symbols have the same meanings as defined above as a substituent on A with a carbonylating agent in a suitable solvent.

The reaction can be carried out in the same manner as in the above (E10).

(E13) The compound having an optionally substituted carbamoyl group as a substituent on A can be prepared by condensing a compound having a carboxyl group as a substituent on A with a desirable amine.

The condensation can be carried out using a condensing agent in a suitable solvent. A conventional condensing agent such as dicyclohexylcarbodiimide, 1-ethyl-3-(3-dimethylaminopropyl)carbodiimide, carbonyldiimidazole, and the like can be preferably used.

Additionally, the condensation can be more preferably carried out by adding an activating agent including 1-hydroxybenzotriazole, 1-hydroxysuccinimide, and the like.

(E14) The compound having a group of the formula:

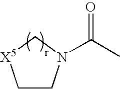

wherein the symbols have the same meanings as defined above as a substituent on A can be prepared by condensing a compound having a carboxyl group as a substituent on A with a compound of the formula:

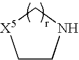

wherein the symbols have the same meanings as defined above.

The reaction can be carried out in the same manner as in the above (E13).

(E15) The compound having a tetrazolyl group as a substituent on A can be prepared by reacting a compound having a cyano group as a substituent on A with an alkaline metal azide in the presence of an acid in a suitable solvent.

The alkaline metal azide includes sodium azide, lithium azide, and the like.

An ammonium salt of a halogenated hydrogen including ammonium chloride can be preferably used as the acid.

(E16) The compound having an optionally substituted alkyl tetrazolyl group as a substituent on A can be prepared by alkylating a compound having a tetrazolyl group as a substituent on A.

The alkylation can be carried out in the same manner as in the above (EA).

(E17) The compound having an optionally substituted amino group or a group of the formula:

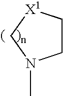

wherein the symbols have the same meanings as defined above as a substituent on A can be prepared by reacting a compound having a halogen atom or an optionally substituted alkylsulfonyloxy group as a substituent on A with a corresponding amine or a compound of the formula:

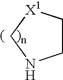

wherein the symbols have the same meanings as defined above.

The reaction can be preferably carried out in the presence or absence of a base in a suitable solvent.

(E18) The compound having an optionally substituted alkylamino group or a group of the formula:

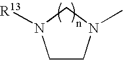

wherein R¹³ is an alkyl group optionally substituted by a hydroxy group, an alkoxycarbonyl group, a morpholinyl group or a phenyl group, and n has the same meaning as defined above, as a substituent on A can be obtained by reacting a compound having an amino group or a group of the formula:

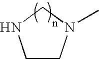

wherein the symbols have the same meanings as defined above as a substituent on A with a corresponding alkyl halide or a corresponding sulfonic acid alkyl esters.

The sulfonic acid alkyl esters including methanesulfonic acid ester, toluenesulfonic acid ester, trifluoromethanesulfonic acid ester, and the like can be preferably used.

The reaction can be preferably carried out in the presence or absence of a base in a suitable solvent.

(E19) The compound having a group of the formula:

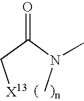

wherein X¹³ is O or NH, and the other symbol has the same meaning as defined above as a substituent on A, can be prepared by ring-closing a compound having a group of the formula:

Z³-(CH₂)ₙ—X¹³—CH₂—CONH—

wherein Z³ is a leaving group and the other symbols have the same meanings as defined above, as a substituent on A.

The reaction can be preferably carried out in the presence or absence of a base in a suitable solvent.

(E20) The compound having a carboxyl group as a substituent on A can be prepared by hydrolyzing a compound having an alkoxycarbonyl group as a substituent on A.

The hydrolysis can be carried out by treating a starting compound with a base or an acid in a suitable solvent according to a conventional manner. An alkaline metal hydroxide can be preferably used as the base.

(E21) The compound containing a carboxyl group as a substituent on A can be prepared by hydrolyzing a compound containing a cyano group as a substituent on A.

The hydrolysis can be carried out by treating a starting compound with an acid or a base in a suitable solvent.

(E22) The compound containing a carbamoyl group as a substituent on A can be prepared by hydrolyzing a compound containing a cyano group as a substituent on A.

The hydrolysis can be carried out by treating a starting compound with an acid or a base in a suitable solvent.

(E23) The compound having a carboxyalkyl group as a substituent on A can also be prepared by catalytically reducing a compound having a carboxyalkenyl group, a benzyloxycarbonylalkenyl group or a benzyloxycarbonylalkyl group as a substituent on A.

The catalytic reduction can be carried out in the same manner as in the above (E6).

(E24) The compound having a hydroxy group as a substituent on A can be prepared by hydrolyzing a compound wherein A has an alkanoyloxy group.

The hydrolysis can be carried out in the same manner as in the above (E20).

(E25) The compound containing sulfoxide (SO) or sulfone (SO₂) in a substituent on A can be prepared by oxidizing a compound having S in a substituent on A (e.g., a compound having a thiomorpholinyl group or an alkylsulfanylalkyl group as a substituent on A).

The oxidation can be carried out by treating a starting compound with an oxidizing agent in a suitable solvent.

Peroxides such as hydrogen peroxide, m-chloroperbenzoic acid, acetyl hydroperoxide, and the like can be preferably used as the oxidizing agent.

(E26) The compound containing N-oxide in a substituent on A can be prepared by oxidizing a compound having N in a substituent on A (e.g., a compound having a pyridyl group as a substituent on A).

The oxidation can be carried out in the same manner as in the above (E25).

(E27) The compound having a 1,2-dihydroxyalkyl group as a substituent on A can be prepared by treating a compound having an alkyl group substituted by mono- or di-alkyldioxolanyl group as a substituent on A with an acid in a suitable solvent.

A strongly acidic resin can also be preferably used as the acid, in addition to those previously described.

(E28) The compound having an alkyl group substituted by a hydroxy group and an optionally substituted alkoxy group as substituents on A can be prepared by reacting a compound having an oxiranylalkyl group as a substituent on A with an alkaline metal salt of the corresponding alcohol in a suitable solvent.

The alkaline metal salt of alcohol includes a lithium salt, a sodium salt, a potassium salt, and the like.

(E29) The compound having an alkyl group substituted by a hydroxy group and an amino group, or an alkyl group substituted by a hydroxy group and an optionally substituted mono- or di-alkylamino group as substituents on A can be prepared by reacting a compound having an oxiranylalkyl group as a substituent on A with ammonia or a corresponding mono- or di-alkylamines in a suitable solvent.

(E30) The compound having a hydroxycarbamimidoyl group as a substituent on A can be prepared by reacting a compound having a cyano group as a substituent on A with hydroxylamine or a salt thereof in a suitable solvent. Any solvent which does not disturb the reaction can be used, and as such, the solvent described in the above (EA) can be preferably used.

(E31) The compound having an oxodihydrooxadiazolyl group as a substituent on A can be prepared by reacting a compound having a hydroxycarbamimidoyl group as a substituent on A with a carbonylating agent in a suitable solvent in the presence or absence of a base.

The same carbonylating agent as that described in the above (E10) can be used.

(E32) The compound having a sulfo group as a substituent on A can be prepared by hydrolyzing a compound having an alkoxycarbonylalkylsulfonyl group as a substituent on A.

The hydrolysis can be carried out in the same manner as in the above (E20).

(E33) The compound having a sulfamoyl group as a substituent on A can be prepared by condensing a compound having a sulfo group as a substituent on A with a desirable amine.

The condensation can be carried out by treating a compound having a sulfo group as a substituent on A with a halogenating agent in a suitable solvent, followed by reacting the resulting compound with a desirable amine in the presence or absence of a base.

A conventional halogenating agent including thionyl halide, phosphorus oxyhalide, or the like can be used.

(E34) The compound having a hydroxyalkyl group as a substituent on A can be prepared by reducing a compound having a carboxyalkyl group as a substituent on A, or by converting the carboxyl group into an acid anhydride or an ester and then reducing the resulting compound.

A process for conversion into an acid anhydride can be carried out by reacting a starting compound with a halogenated alkyl formate in a suitable solvent in the presence of a base.

A process for conversion into an ester can be carried out by reacting a starting compound with an alcohol in the presence of a condensing agent in a suitable solvent. This process can be carried out in the same manner as in (E33) except that a desirable alcohol is used in place of amine.

The reduction can be carried out by treating the resulting compound with a reducing agent in a suitable solvent.

Boron hydrides (e.g. sodium borohydride), aluminum hydrides (lithium aluminum hydride, diisobutylaluminum hydride, and the like) can be preferably used as the reducing agent.

(E35) The compound having an aromatic group substituted by a cyano group as a substituent on R¹, optionally having one to three heteroatoms independently selected from oxygen atom, sulfur atom and nitrogen atom (hereinafter, referred to as “an aromatic group”), can also be prepared by cyanating a compound having an aromatic group substituted by a halogen atom as a substituent on R¹.

The cyanation can be carried out in the same manner as in the above (EE).

(E36) The compound wherein A is a hydrogen atom can be prepared by acid-treatment or reduction of a compound wherein A is a tert-butoxycarbonyl group or a benzyloxycarbonyl group.

The acid-treatment can be carried out in the same manner as in the above (E27) and the reduction can be carried out in the same manner as in the above (E23).

(E37) The compound wherein A is an optionally substituted alkoxycarbonyl group, or an optionally substituted carbamoyl group can be prepared by reacting a compound wherein A is a hydrogen atom with a carbonylating agent, or a desirable alcohol or a desirable amine in a suitable solvent.

The reaction can be carried out in the same manner as in the above (E10).

(E38) The compound having an amino group as a substituent on A can be prepared by undergoing a Curtius rearrangement reaction of a compound having a carboxyl group as a substituent on A.

Curtius rearrangement reaction can be carried out using a conventional azidating agent (e.g., diphenylphosphorylazide, and the like) in a suitable solvent in the presence of a base.

The reaction may also be carried out by adding alcohols to provide a compound having an optionally substituted alkoxycarbonylamino group as a substituent on A, followed by removing the alkoxycarbonyl group.

The removal of the alkoxycarbonyl group can be carried out in a conventional manner such as an acid-treatment or a reduction depending on the type of alkoxycarbonyl group to be removed. The acid-treatment can be carried out in the same manner as in the above (E27) and the reduction can be carried out in the same manner as in the above (E23).

(E39) The compound having a hydroxy group as a substituent on A can be prepared by catalytically reducing a compound having a benzyloxy group as a substituent on A. The reduction can be carried out in the same manner as in the above (E23).

(E40) The compound having an oxo group as a substituent on A can be prepared by oxidizing a compound having a hydroxy group as a substituent on A.

The oxidation can be carried out by using an oxidizing agent in a suitable solvent.

A conventional oxidizing agent can be used as the oxidizing agent, such as chromate-pyridine complex, pyridinium chlorochromate, pyridinium dichromate, Dess-Martin reagent (1,1,1-tris(acetoxy)-1,1-dihydro-1,2-benziodoxol-3-(1H)-one), dimethyl sulfoxide, and the like.

(E41) The compound containing an optionally substituted alkoxy group as a substituent on A can be prepared by alkylating a compound containing an oxo group or a hydroxy group as a substituent on A.

The alkylation can be carried out by using a corresponding compound in the same manner as in the above (EA).

(E41′) The compound containing an optionally substituted heterocyclyloxy group or an optionally substituted aryloxy group as a substituent on A can be prepared by coupling a compound containing a hydroxy group as a substituent on A with a corresponding aryl compound or a heterocyclic compounds which have a leaving group.

The coupling can be carried out in the same manner as in the above (EC).

The leaving group have the same meaning as defined above (EH).

(E42) The compound having an optionally substituted alkanoylamino group as a substituent on A can be prepared by condensing a compound having an amino group as a substituent on A with a corresponding carboxylic acid or a reactive derivative thereof.

The condensation with the corresponding carboxylic acid can be preferably carried out in a suitable solvent in the presence of a condensing agent. The reaction can be carried out in the same manner as in the above (E13).

Additionally, the condensation with the reactive derivative of the corresponding carboxylic acid can be carried out in a suitable solvent or solvent-free in the presence or absence of a base.

The reactive derivative includes an acid halide, an acid anhydride, an activated ester, an activated amide, and the like.

(E43) The compound having a group of the formula:

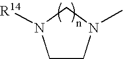

wherein R¹⁴ is an alkanoyl group optionally substituted by a hydroxy group or an alkoxy group, and n has the same meaning as defined above, as a substituent on A can be prepared by condensing a compound of a group of the formula:

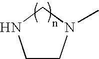

wherein the symbol has the same meaning as defined above, as a substituent on A with a corresponding carboxylic acid or a reactive derivative thereof.

The reaction can be carried out in the same manner as in the above (E42).

(E44) The compound having a maleimide group as a substituent on A can be prepared by reacting a compound having an amino group as a substituent on A with a maleic anhydride. The reaction can be carried out in a suitable solvent.

(E45) The compound having an alkyl group substituted by a pyridyl group and a hydroxy group as substituents on A can be prepared by reacting a compound having an alkyl group substituted by a pyridyl group of which nitrogen atom is oxidized as a substituent on A with a trifluoroacetic anhydride. The reaction can be carried out in a suitable solvent.

(E46) The compound having a halogen atom as a substituent on A can be prepared by treating a compound having a hydroxy group as a substituent on A with a halogenating agent.

As the halogenating agent, a conventional halogenating agent including thionyl chloride, phosphorus oxychloride, as well as carbon tetrahalide (e.g., carbon tetrachloride, carbon tetrabromide, and the like) and phosphines (e.g., triphenylphosphine, tritolylphosphine, triethylphosphine, and the like) can be preferably used.

(E46′) The compound having a halogen atom as a substituent on A can be prepared by treating a compound with a halogenating agent. As the halogenating agent, a conventional halogenating agent such as bromine, N-bromosuccinimide, and the like can be preferably used.

(E47) The compound having a cyanoalkyl group as a substituent on A can be prepared by reducing a compound having a cyanoalkenyl group as a substituent on A.

The reduction can be carried out by treating a starting compound with a reducing agent or by catalytically reducing in a suitable solvent.

Any reducing agent can be used subject that it reduces only a double bond without affecting a cyano group. For example, sodium bis(2-methoxyethoxy)aluminum hydride in the presence of a copper bromide can be preferably used.

The catalytic reduction can be carried out in the same manner as in the above (E23).

(E48) The compound (1) having a hydroxyalkyl group as a substituent on A can be prepared by reducing a compound having a formyl group as a substituent on A.

The reduction can be carried out by treating a starting compound with a reducing agent in a suitable solvent.

The reaction can be carried out in the same manner as in the process for reducing in the above (E34).

(E49) The compound wherein a substituent on B is a hydroxy group can be prepared by demethylating a compound wherein the substituent on B is a methoxy group.

The demethylation can be carried out by treating a starting compound with a demethylating agent in a suitable solvent.

A conventional reagent including trimethylsilyl iodide, hydrogen bromide/acetic acid, boron tribromide, concentrated sulfuric acid, and the like can be used as the demethylating agent.

(E50) The compound wherein a substituent on B is an optionally substituted alkoxy group can be prepared by alkylating a compound wherein the substituent on B is a hydroxy group.

The alkylation can be carried out in the same manner as in the above (EA).

(E51) The compound wherein a substituent on B is an optionally substituted alkylsulfonyloxy group can be prepared by alkylsulfonylating a compound wherein the substituent on B is a hydroxy group.

The alkylsulfonylation can be carried out by reacting a corresponding alkylsulfonyl halide or a corresponding alkylsulfonic anhydride in a suitable solvent in the presence or absence of a base.

(E52) The compound wherein a substituent on B is a cyano group can be prepared by cyanating a compound wherein the substituent on B is an optionally substituted alkylsulfonyloxy group.

The cyanation can be carried out in the same manner as in the above (EE).

(E53) The compound wherein a substituent on B is an aminoalkyl group can be prepared by reducing a compound wherein the substituent on B is a cyano group.

The reduction can be carried out in the same manner as in the above (E6).

(E54) The compound wherein a substituent on B is an alkyl group can be prepared by alkylating a compound wherein the substituent on B is an optionally substituted alkylsulfonyloxy group.

The alkylation can be carried out by reacting alkyl aluminums in the presence of a palladium catalyst, a silver catalyst and a copper catalyst in a suitable solvent.

Tetrakis(triphenylphosphine)palladium as the palladium catalyst, silver carbonate as the silver catalyst, copper (I) chloride as the copper catalyst can be preferably used.

(E54′) The compound wherein a substituent on B is an optionally substituted alkyl group can be prepared by catalytically reducing a compound wherein a substituent on B is an optionally substituted alkenyl group. The reaction can be carried out in the same manner as in the above (E6).

(E55) The compound having an imidazolinyl group or an oxazolinyl group as a substituent on A can be prepared by (i) reacting a compound containing a cyano group as a substituent on A with a desirable alcohol in the presence of an acid in a suitable solvent or solvent-free to provide a compound containing an alkoxycarbonimidoyl group as a substituent on A, and (ii) reacting the compound containing an alkoxycarbonimidoyl group as a substituent on A with 2-aminoethanol or ethylene diamine in a suitable solvent or solvent-free.

(E56) The compound having a carboxyl group as a substituent on A can be prepared by (i) oxidizing a compound containing a hydroxyalkyl group as a substituent on A in the same manner as in the above (E40) to provide a compound containing an oxo group as a substituent on A, and (ii) oxidizing the compound containing an oxo group as a substituent on A.

The oxidization for the second process can be carried out by using an oxidizing agent in a suitable solvent. Sodium chlorite, Silver(I) oxide, Sodium periodate and the like can be preferably used as the oxidizing agent.

(E57) The compound having a carboxyl group as a substituent on A can be directly prepared by oxidizing a compound containing a hydroxyalkyl group as a substituent on A.

The oxidization can be carried out by using Jones reagent, potassium permanganate, and the like as the oxidizing agent.

(E58) The compound wherein A is hydrogen atom can be prepared by treating a compound wherein A is ethoxycarbonyl group with a silyl halide or a base. Trimethylsilyl iodide can be preferably used as the silyl halide. Sodium hydroxide can be preferably used as the base.

In each process for preparing a compound (1) described above, when protection of a functional group contained in any compound is needed, the protection can be carried out in a conventional manner as appropriate. General statement related to protecting groups and their use is provided by Greene, Protective Groups in Organic Synthesis, John Wiley and Sons, New York, 1991.

When an amino group is protected by a benzyloxycarbonyl group, the protecting group can be removed by a catalytic reduction under hydrogen atmosphere in a suitable solvent.

When a hydroxy group is protected by a benzyl group, the protecting group can also be removed by a catalytic reduction in a similar manner as above.

When an amino group is protected by a tert-butoxycarbonyl group, the protecting group can be removed by treating a starting compound with an acid (e.g., hydrochloric acid, trifluoroacetic acid, toluenesulfonic acid, and the like) in a suitable solvent.

When a hydroxy group is protected by a tetrahydropyranyl group, the protecting group can also be removed by treating a starting compound with an acid in a similar manner as above.

Other substituents of the present compound (1) can also be converted in the same manner as in the reactions (E1) to (E58) for conversion of the above groups.

### [Preparation of a Compound (6′)]

The compound (6′) can be prepared according to the following method (a) or (b).

(a) The compound (6) can be prepared by reacting the compound of a general formula (10):

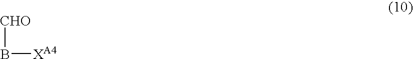

wherein X^{A4} is a leaving group and the other symbols have the same meanings as defined above, with the compound of a general formula (11):

H—R² (11)

wherein the symbol has the same meaning defined above, in the same manner as in the above process I-1 or process I′-1.

(b) The compound (6′) can be prepared by reacting the compound of a general formula (12):

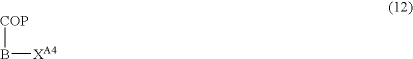

wherein P is a protecting group for a carboxyl group and the other symbols have the same meanings as defined above, with the compound (11) in the same manner as in the above process I-1 or process I′-1, followed by reducing the resulting compound to provide the compound (6″) and further oxidizing the resulting compound.

The reduction and oxidation in the above methods can be carried out by the conventional method.

### [Preparation of a Compound (2)]

The compound (2) can be prepared by oximating the compound (6′) to provide the compound of a general formula (13):

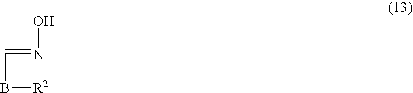

wherein the symbols have the same meanings as defined above, followed by reducing the resulting compound.

The oximation can be carried out by the conventional oximation methods, for example, by treating the compound (6′) with a salt of hydroxylamine in the presence of an acid or a base such as alkaline metal hydroxide, sodium acetate or pyridine in an alcohol, acetic acid or pyridine. Also any acidic material can be used as the agent for preparing a salt of hydroxylamine, for example, a mineral acid (e.g. sulfuric acid, phosphoric acid, hydrogen bromide and hydrogen iodide), and organic acid (e.g. acetic acid, oxalic acid, trichloroacetic acid, methanesulfonic acid, p-toluenesulfonic acid, 1,5-naphthalenedisulfonic acid).

The subsequent reduction reaction can be carried out in a conventional manner.

The compound (2) can also be prepared from the compound (6″) using the method of Gabriel synthesis, described in detail in Mitsunobu, O. Comp. Org. Syn. 1991, 6, 79-85.

### [Preparation of a Compound (6′″″)]

The compound (6′″″) can be prepared from the compound of a general formula (14):

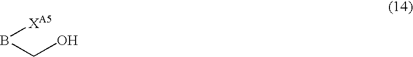

wherein X^{A5} is a leaving group and the other symbols have the same meanings as defined above, by a conventional insertion reaction of carbon monoxide with a transition metal catalyst.

The reaction can be carried out in an aprotic solvent such as tetrahydrofuran or DMF, and the like. Preferred transition metal includes, for example, a salt form of palladium such as palladium (II)-acetate, and the like or a palladium (0) compound such as tetrakis(triphenylphosphine)palladium, and the like. This kind of an insertion reaction of carbon monoxide can be carried out by the method described in detail in J. Org. Chem. 1992, 57, 5979 or “Organometallic compound-Synthesis and Application-(Tokyo Kagaku Dozin Co., Ltd.), Metal-catalyzed Cross-coupling Reaction (WILLY-VCH), Handbook of Palladium-Catalyzed Organic Reactions (Academic Press)”, and the like.

The compound (1-a) wherein A¹ is tetrazolyl group and A² is a hydrogen atom can be prepared by cyanidating the above compound (8′) to provide the compound of a general formula (8″″):

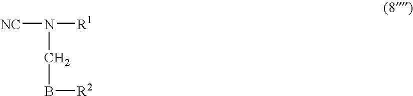

wherein the symbols have the same meanings as defined above, followed by reacting with an alkaline metal azide.

The cyanidation can be carried out by reacting a starting compound with cyanogen halide in the presence of a base in a suitable solvent.

The cyanogen halide is preferably cyanogen bromide.

The conventional base can be preferably used as a base, such as alkaline metal carbonate (e.g. potassium carbonate) or an alkaline metal bicarbonate (e.g. sodium bicarbonate).

Any solvent which dose not disturb the reaction can be used as a solvent and the solvent illustrated in the above method I can be preferably used.

The conversion of a cyano group into a tetrazolyl group can be achieved by reacting the compound having a cyano group with an alkaline metal azide in the presence of an acid in a suitable solvent.

The alkaline metal azide includes sodium azide and lithium azide, and the like.

The ammonium salt of halogenated hydrogen such as ammonium chloride can be preferably used as an acid.

In addition, for performing the above methods, there can be referred to PCT International Publication WO04/020393 pamphlet, WO05/100298 pamphlet and JP. 2003-221376 A.

The methods for preparation of the compound (1) are applicable to preparation of the corresponding compounds of formula (I-A), (I-B) and (1-2).

Many of starting materials and reagents for preparation of the aforementioned compound of the formula I are either commercially available or disclosed in literatures, or can be readily prepared by a method that is disclosed in literatures or used generally in the organic synthesis.

Experiment

The inhibitory activity of the compounds (1) of the present invention against CETP was tested in this experiment.

Preparation of Acceptor Microemulsion

A solution of 1-Palmitoyl-2-oleoyl-sn-glycero-3-phosphocholine (3.5 mg), cholesteryl oleate (3 mg) and triolein (0.7 mg) in chloroform was mixed and lipid was air-dried under nitrogen gas to remove solvent. 1,4-Dioxane (0.25 ml) was then added and the mixture was stirred for dissolution. The resultant lipid solution (0.2 ml) was slowly injected under the surface of Tris-saline-EDTA (TSE) buffer solution [10 mM Tris/HCl (pH 7.4), 0.15M NaCl, 2 mM EDTA] (10 ml) with Hamilton syringe, while sonicating in ice-bath. After 1-hour-sonication in ice-bath, the solution was stored at 4° C.

Preparation of Donor Microemulsion

A solution of egg PC (phosphatidylcholine) (0.33 mg) and BODIPY-CE™ (Invitrogen Corporation) (0.62 mg) in chloroform was mixed. After removing solvent by air-drying lipid under nitrogen gas, TSE buffer solution (3 ml) was added and the solution was sonicated in ice-bath. This solution was filtered to sterilize through 0.22 μm filter and stored at 4° C.

Inhibitory Activity Against CETP In Vitro

A test solution was prepared using dimethyl sulfoxide as a solvent. Plasma from a healthy volunteer was diluted to 0.64% with TSE buffer, and to the resultant plasma solution (187 μl) was added a test solution (3 μl) or the solvent alone followed by incubation at 37° C. for 24 hours. After addition of TSE buffer solution (10 μl) containing 5% donor micro-emulsion and 5% acceptor microemulsion, the mixture was incubated at 37° C. for 3 hours. Before and after the incubation, the fluorescence intensity was measured at Ex.550 nm/Em.600 nm. CETP activity was defined as the difference between the measurements obtained before incubation and after incubation. The decreasing rate of the difference in the sample was defined as the inhibition rate of CETP activity.

### EXAMPLES

The present invention is illustrated in more detail by Examples and Reference Examples, but the present invention should not be construed to be limited thereto. Meanwhile, in the following Tables, the symbol “Me” means methyl group and the symbol “TFA” means trifluoroacetic acid.

### Example 1

(1) 2-Chloroquinoline-3-carboxaldehyde (650 mg) and cyclopropylmethyl-propyl-amine (576 mg) are dissolved in toluene (5 ml), and thereto is added potassium carbonate (1.41 g) and the mixture is stirred at 120° C. overnight. The reaction solution is cooled to room temperature, and thereto are added ethyl acetate and water, and the mixture is separated, and the organic layer is washed with a saturated brine, dried over magnesium sulfate and concentrated under reduced pressure. The residue is dissolved in ethanol (5 ml), and thereto is added sodium borohydride (128 mg) at room temperature and the mixture is stirred for 1 hour. To the reaction solution are added saturated aqueous ammonium chloride solution and ethyl acetate, and the mixture is separated, and the organic layer is washed with a saturated brine, dried over magnesium sulfate and concentrated under reduced pressure. The resulting residue is purified by silica gel column chromatography (hexane:ethyl acetate=1:0→3:1) to give-[2-(cyclopropylmethyl-propyl-amino)-quinolin-3-yl]-methanol (350 mg). MS (m/z): 271 [M+H]⁺

(2) [2-(Cyclopropylmethyl-propyl-amino)-quinolin-3-yl]-methanol (100 mg) is dissolved in methylene chloride (1 ml) and thereto is added thionyl chloride (30 μl) under ice-cooling, and the mixture is stirred at the same temperature for 10 minutes. To the reaction solution are added a saturated aqueous sodium bicarbonate solution and ethyl acetate, and the mixture is separated, and the organic layer is washed with a saturated brine, dried over magnesium sulfate, and concentrated under reduced pressure. Ethyl 4-[2-(3,5-bis-trifluoromethyl-benzylamino)-pyrimidin-5-yloxy]-butyrate (167 mg) is dissolved in N,N-dimethylformamide (0.5 ml) and thereto is added sodium hydride (60%) (14.8 mg) under ice-cooling, and the mixture is stirred for 15 minutes. Thereto is added a solution of a residue obtained above in N,N-dimethylformamide (0.5 ml) and the mixture is stirred at room temperature for 1 hour. To the reaction solution are added ethyl acetate and water, and the mixture is separated, and the organic layer is washed with a saturated brine, dried over magnesium sulfate and concentrated under reduced pressure. The resulting residue is purified by silica gel column chromatography (hexane:ethyl acetate=49:1→9:1) to give ethyl 4-(2-{(3,5-bis-trifluoromethyl-benzyl)-[2-(cyclopropylmethyl-propyl-amino)-quinolin-3-ylmethyl]-amino}-pyrimidin-5-yloxy)-butyrate (35 mg). MS (m/z): 704 [M+H]⁺

(3) Ethyl 4-(2-{(3,5-bis-trifluoromethyl-benzyl)-[2-(cyclopropylmethyl-propyl-amino)-quinolin-3-ylmethyl]-amino}-pyrimidin-5-yloxy)-butyrate (34 mg) is dissolved in ethanol (0.5 ml) and thereto is added a 2N-aqueous sodium hydroxide solution (72 μl) and the mixture is stirred at room temperature overnight. The reaction solution is concentrated under reduced pressure and to the residue are added ethyl acetate and a dilute hydrochloric acid, and the mixture is separated and the organic layer is washed with a saturated brine, dried over magnesium sulfate and concentrated under reduced pressure to give 4-(2-{(3,5-bis-trifluoromethyl-benzyl)-[2-(cyclopropylmethyl-propyl-amino)-quinolin-3-ylmethyl]-amino}-pyrimidin-5-yloxy)-butyric acid (30 mg). MS (m/z): 676 [M+H]⁺

### Example 2

The corresponding starting compound is treated in a similar manner to Example 1 to give the compound as listed in Table 1.

### Example 3

(1) 2-Chloroquinoline-3-carboxaldehyde (330 mg) is dissolved in 1,2-dimethoxy-ethane (7.5 ml), and thereto are added a 2M-aqueous sodium carbonate solution (1.72 ml), water (2.5 ml) and ethanol (2.5 ml), and the mixture is degassed under reduced pressure and flushed with nitrogen gas. Thereto are added 5-isopropyl-2-methoxyphenylboronic acid (417 mg) and tetrakis(triphenylphosphine)palladium (199 mg), and the mixture is flushed with nitrogen gas again, and the mixture is set in the microwave instrument, heated to 150° C. by irradiating microwave of 200 W and stirred for 15 minutes. The reaction solution is cooled to room temperature, and thereto are added diethyl ether and water and the mixture is separated, and the organic layer is washed with a saturated brine, dried over magnesium sulfate and concentrated under reduced pressure. The residue is purified by silica gel column chromatography (hexane:ethyl acetate=19:1→3:1) to give 2-(5-isopropyl-2-methoxy-phenyl)-quinoline-3-carboxaldehyde (493 mg). MS (m/z): 306 [M+H]⁺

(2) 2-(5-Isopropyl-2-methoxy-phenyl)-quinoline-3-carboxaldehyde (470 mg) is dissolved in ethanol (5 ml), and thereto is added sodium borohydride (58 mg) at room temperature and the mixture is stirred overnight. To the reaction solution are added a saturated aqueous ammonium chloride solution and diethylether, and the mixture is separated, and the organic layer is washed with a saturated brine, dried over magnesium sulfate and concentrated under reduced pressure. The residue is purified by silica gel column chromatography (hexane:ethyl acetate=4:1→1:1) to give [2-(5-isopropyl-2-methoxy-phenyl)-quinolin-3-yl]-methanol (415 mg). MS (m/z): 308 [M+H]⁺

(3) [2-(5-Isopropyl-2-methoxy-phenyl)-quinolin-3-yl]-methanol (409 mg) is dissolved in methylene chloride (25 ml) and thereto is added thionyl chloride (107 μl) under ice-cooling, and the mixture is stirred at room temperature for 15 minutes and concentrated under reduced pressure. The residue and (3,5-bis-trifluoromethyl-benzyl)-(5-bromopyrimidin-2-yl)-amine (532 mg) are dissolved in N,N-dimethylformamide (5 ml) and thereto is added sodium hydride (62%) (103 mg) under ice-cooling and the mixture is stirred at room temperature overnight. To the reaction solution are added diethyl ether and water and the mixture is separated, and the organic layer is washed with a saturated brine, dried over magnesium sulfate and concentrated under reduced pressure. The resulting residue is purified by silica gel column chromatography (hexane:ethyl acetate=19:1→4:1) to give (3,5-bis-trifluoromethyl-benzyl)-(5-bromo-pyrimidin-2-yl)-[2-(5-isopropyl-2-methoxy-phenyl)-quinolin-3-ylmethyl]amine (450 mg). MS (m/z): 689/691 [M+H]⁺

### Example 4

(1) (3,5-Bis-trifluoromethyl-benzyl)-(5-bromo-pyrimidin-2-yl)-[2-(5-isopropyl-2-methoxy-phenyl)-quinolin-3-ylmethyl]-amine (200 mg) is dissolved in toluene (3 ml), and thereto are added tris(dibenzylideneacetone)dipalladium (26.6 mg), sodium tert-butoxide (41.8 mg), 2-(di-tert-butylphosphino)biphenyl (34.6 mg) and ethyl piperidine-4-carboxylate (65 μl) and the mixture is stirred at room temperature under nitrogen flow overnight. To the reaction solution is added a saturated brine, and the mixture is extracted with diethyl ether. The organic layer is washed with a saturated brine, dried over magnesium sulfate and concentrated under reduced pressure. The resulting residue is purified by silica gel column chromatography (hexane:ethyl acetate=43:7→73:27) to give ethyl 1-(2-{(3,5-bis-trifluoromethyl-benzyl)-[2-(5-isopropyl-2-methoxy-phenyl)-quinolin-3-ylmethyl]-amino}-pyrimidin-5-yl)-piperidine-4-carboxylate (85 mg). MS (m/z): 766 [M+H]⁺

(2) Ethyl 1-(2-{(3,5-bis-trifluoromethyl-benzyl)-[2-(5-isopropyl-2-methoxy-phenyl)-quinolin-3-ylmethyl]-amino}-pyrimidin-5-yl)-piperidine-4-carboxylate (80 mg) is dissolved in ethanol (3 ml), and thereto is added a 2N-aqueous sodium hydroxide solution (157 μl) and the mixture is stirred at room temperature overnight. To the reaction solution are added diethyl ether and water and the pH of aqueous layer is made pH 4 with a 1N-hydrochloric acid, and the mixture is separated, and the organic layer is washed with a saturated brine, dried over magnesium sulfate and concentrated under reduced pressure. The resulting residue is purified by silica gel column chromatography (chloroform:methanol=1:0→93:7) to give 1-(2-{(3,5-bis-trifluoromethyl-benzyl)-[2-(5-isopropyl-2-methoxy-phenyl)-quinolin-3-ylmethyl]-amino}-pyrimidin-5-yl)-piperidine-4-carboxylic acid (75 mg). MS (m/z): 738 [M+H]⁺

### Example 5

(1) (3,5-Bis-trifluoromethyl-benzyl)-(5-bromo-pyrimidin-2-yl)-[2-(5-isopropyl-2-methoxy-phenyl)-quinolin-3-ylmethyl]-amine (165 mg), [1,1′-bis(diphenylphosphino)ferrocene]dichloropalladium methylene chloride complex (39 mg), potassium acetate (70 mg) and bis(pinacolato)diboron (122 mg) are dissolved in dimethylsulfoxide (1.6 ml), and the mixture is heated to 80° C. under nitrogen atmosphere and stirred for 1 hour. The reaction solution is cooled to room temperature and thereto are added water and diethyl ether, and the mixture is separated, and the organic layer is washed with a saturated brine, dried over magnesium sulfate and concentrated under reduced pressure. The resulting residue is dissolved in tetrahydrofuran (5 ml) and thereto is added dropwise a 30% aqueous hydrogen peroxide solution (0.5 ml) under ice-cooling. One hour thereafter, thereto is added a saturated aqueous sodium thiosulfate solution under ice-cooling to consume the excess hydrogen peroxide, and thereto are added water and ethyl ether and the mixture is separated. The organic layer is washed with a saturated brine, dried over magnesium sulfate and concentrated under reduced pressure. The resulting residue is purified by silica gel column chromatography (hexane:ethyl acetate=4:1→67:33) to give 2-{(3,5-bis-trifluoromethyl-benzyl)-[2-(5-isopropyl-2-methoxy-phenyl)-quinolin-3-ylmethyl]-amino}-pyrimidin-5-ol (116 mg). MS (m/z): 627 [M+H]⁺

(2) 2-{(3,5-Bis-trifluoromethyl-benzyl)-[2-(5-isopropyl-2-methoxy-phenyl)-quinolin-3-ylmethyl]-amino}-pyrimidin-5-ol (110 mg) and ethyl 4-bromobutyrate (28 μl) are dissolved in N,N-dimethylformamide (2 ml) and thereto is added potassium carbonate (29 mg), and the mixture is stirred at room temperature overnight. Thereto are added diethyl ether and a saturated brine, and the mixture is separated, and the organic layer is washed with a saturated brine, dried over magnesium sulfate and concentrated under reduced pressure. The resulting residue is purified by silica gel column chromatography (hexane:ethyl acetate=41:9→7:3) to give ethyl 4-(2-{(3,5-bis-trifluoromethyl-benzyl)-[2-(5-isopropyl-2-methoxy-phenyl)-quinolin-3-ylmethyl]-amino}-pyrimidin-5-yloxy)-butyrate (71 mg). MS (m/z): 741 [M+H]⁺

(3) Ethyl 4-(2-{(3,5-bis-trifluoromethyl-benzyl)-[2-(5-isopropyl-2-methoxy-phenyl)-quinolin-3-ylmethyl]-amino}-pyrimidin-5-yloxy)-butyrate (65 mg) is dissolved in ethanol (3 ml), and thereto is added a 2N-aqueous sodium hydroxide solution (132 μl) and the mixture is stirred at room temperature overnight. Thereto are added diethyl ether and water, and the pH of aqueous layer is made pH 4 with a 1N-hydrochloric, and the mixture is separated, and the organic layer is washed with a saturated brine, dried over magnesium sulfate and concentrated under reduced pressure. The resulting residue is purified by silica gel column chromatography (chloroform:methanol=1:0→47:3) to give 4-(2-{(3,5-bis-trifluoromethyl-benzyl)-[2-(5-isopropyl-2-methoxy-phenyl)-quinolin-3-ylmethyl]-amino}-pyrimidin-5-yloxy)-butyric acid (51 mg). MS (m/z): 713 [M+H]⁺

### Example 6

(1) To toluene (50 ml) are added 2-chloroquinoline-3-carboxaldehyde (8.97 g) and cyclopropylmethyl-propyl-amine (7.95 g), followed by an addition of potassium carbonate (19.4 g), and the mixture is stirred at 120° C. for 8 hours. The reaction solution is cooled to room temperature, and thereto is added water and the mixture is separated, and the organic layer is washed with a saturated brine, dried over magnesium sulfate, and concentrated under reduced pressure. The resulting residue is purified by silica gel column chromatography (hexane:ethyl acetate=19:1→14:1) to give 2-(cyclopropylmethyl-propyl-amino)-quinoline-3-carboxaldehyde (9.3 g). MS (m/z): 269 [M+H]⁺

(2) 2-(Cyclopropylmethyl-propyl-amino)-quinoline-3-carboxaldehyde (750 mg), 3,5-bis-trifluoromethyl-benzylamine (883 mg) and acetic acid (479 μl) are dissolved in 1,2-dichloroethane (5 ml), and thereto is added triacetoxy sodium borohydride (1.18 g) at room temperature and the mixture is stirred overnight. To the reaction solution are added methylene chloride and a 1N-aqueous sodium hydroxide solution, and the mixture is separated, and the organic layer is washed with a saturated brine, dried over magnesium sulfate, and concentrated under reduced pressure. The resulting residue is purified by silica gel column chromatography (hexane:ethyl acetate=97:3→17:3) to give {3-[(3,5-bis-trifluoromethyl-benzylamino)-methyl]-quinolin-2-yl}-cyclopropylmethyl-propyl-amine (1.30 g). MS (m/z): 496 [M+H]⁺

(3) {3-[(3,5-Bis-trifluoromethyl-benzylamino)-methyl]-quinolin-2-yl}-cyclopropylmethyl-propyl-amine (979 mg) is dissolved in ethanol (10 ml) and thereto are added sodium bicarbonate (505 mg) and cyanogen bromide (230 mg), and the mixture is stirred at room temperature overnight. Thereto are added diethyl ether and water, and the mixture is separated, and the organic layer is washed with a saturated brine, dried over magnesium sulfate, and concentrated under reduced pressure. The resulting residue is purified by silica gel column chromatography (hexane:ethyl acetate=9:1→3:1) to give (3,5-bis-trifluoromethyl-benzyl)-[2-(cyclopropylmethyl-propyl-amino)-quinolin-3-ylmethyl]-cyanamide (1.02 g). MS (m/z): 521 [M+H]⁺

(4) To N,N-dimethylformamide (10 ml) are added (3,5-bis-trifluoromethyl-benzyl)-[2-(cyclopropylmethyl-propyl-amino)-quinolin-3-ylmethyl]-cyanamide (950 mg), sodium azide (1.186 g) and ammonium chloride (976 mg), and the mixture is stirred at 100° C. overnight. To the reaction solution are added diethyl ether and water, and the mixture is separated, and the organic layer is washed with a saturated brine, dried over magnesium sulfate, and concentrated under reduced pressure. The resulting residue is purified by silica gel column chromatography (chloroform:methanol=1:0→19:1) to give (3-{[(3,5-bis-trifluoromethyl-benzyl)-(2H-tetrazol-5-yl)-amino]-methyl}-quinolin-2-yl)-cyclopropylmethyl-propyl-amine (696 mg). MS (m/z): 564 [M+H]⁺

### Example 7

(1) To N,N-dimethylformamide (1 ml) are added (3-{[(3,5-bis-trifluoromethyl-benzyl)-(2H-tetrazol-5-yl)-amino]-methyl}-quinolin-2-yl)-cyclopropylmethyl-propyl-amine (250 mg), ethyl 2-bromo-2-methyl-propionate (195 μl), a catalytic amount of potassium iodide, and triethylamine (1 ml), and the mixture is stirred at 70° C. overnight. The reaction solution is cooled to room temperature, and thereto are added diethyl ether and water, and the mixture is separated, and the organic layer is washed with a saturated brine, dried over magnesium sulfate, and concentrated under reduced pressure. The resulting residue is purified by NH-silica gel column chromatography (hexane:ethyl acetate=97:3→22:3) to give ethyl 2-(5-{(3,5-bis-trifluoromethyl-benzyl)-[2-(cyclopropylmethyl-propyl-amino)-quinolin-3-ylmethyl]-amino}-tetrazol-2-yl)-2-methyl-propionate (205 mg). MS (m/z): 678 [M+H]⁺

(2) Ethyl 2-(5-{(3,5-bis-trifluoromethyl-benzyl)-[2-(cyclopropylmethyl-propyl-amino)-quinolin-3-ylmethyl]-amino}-tetrazol-2-yl)-2-methyl-propionate (200 mg) is dissolved in a mixed solvent of ethanol (6 ml) and water (1 ml), and thereto is added lithium hydroxide monohydrate (24.8 mg) and the mixture is stirred at room temperature overnight. Thereto are added diethyl ether and water, and the pH of aqueous layer is made 4 with a 1N-hydrochloric acid, and the mixture is separated, and the organic layer is washed with a saturated brine, dried over magnesium sulfate, and concentrated under reduced pressure. The resulting residue is purified by silica gel column chromatography (chloroform:methanol=1:0→9:1) to give 2-(5-{(3,5-bis-trifluoromethyl-benzyl)-[2-(cyclopropylmethyl-propyl-amino)-quinolin-3-ylmethyl]-amino}-tetrazol-2-yl)-2-methyl-propionic acid (172 mg). MS (m/z): 650 [M+H]⁺

### Example 8

(1) (3-{[(3,5-Bis-trifluoromethyl-benzyl)-(2H-tetrazol-5-yl)-amino]-methyl}-quinolin-2-yl)-cyclopropylmethyl-propyl-amine (250 mg), methyl 3-hydroxy-2,2-dimethyl-propionate (94 μl) and triphenylphosphine (193 mg) are dissolved in tetrahydrofuran (5 ml), and thereto is added a solution of 40% diethyl azodicarboxylate in toluene (290 μl) at room temperature. The reaction solution is stirred at room temperature overnight and concentrated under reduced pressure. The resulting residue is purified by silica gel column chromatography (hexane:ethyl acetate=19:1→41:9) to give methyl 3-(5-{(3,5-bis-trifluoromethyl-benzyl)-[2-(cyclopropylmethyl-propyl-amino)-quinolin-3-ylmethyl]-amino}-tetrazol-2-yl)-2,2-dimethyl-propionate (174 mg). MS (m/z): 678 [M+H]⁺

(2) Methyl 3-(5-{(3,5-bis-trifluoromethyl-benzyl)-[2-(cyclopropylmethyl-propyl-amino)-quinolin-3-ylmethyl]-amino}-tetrazol-2-yl)-2,2-dimethyl-propionate (170 mg) is dissolved in a mixed solvent of ethanol (6 ml) and water (1 ml), and thereto is added lithium hydroxide monohydrate (21 mg), and the mixture is stirred at room temperature overnight. The reaction solution is concentrated under reduced pressure, and to the residue is added diethyl ether and water, and the pH of aqueous layer is made pH 4 with a 1N-hydrochloric acid, and the mixture is separated, and the organic layer is washed with a saturated brine, dried over magnesium sulfate, and concentrated under reduced pressure. The resulting residue is purified by silica gel column chromatography (chloroform:methanol=1:0→97:3) to give 3-(5-{(3,5-bis-trifluoromethyl-benzyl)-[2-(cyclopropylmethyl-propyl-amino)-quinolin-3-ylmethyl]-amino}-tetrazol-2-yl)-2,2-dimethyl-propionic acid (153 mg). MS (m/z): 664 [M+H]⁺

TABLE 1

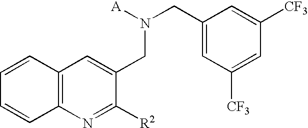

| Ex. | | | Physical |
|---|---|---|---|
| No. | A- | —R² | properties, etc. |
| 1 | 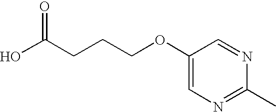 | 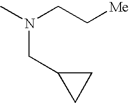 | MS (m/z):676 [M + H]⁺ |
| 2 | 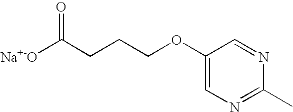 | 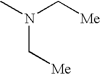 | MS (m/z):634 [M − Na]⁻ |
| 3 | 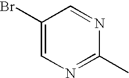 | 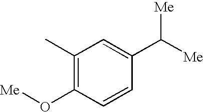 | MS (m/z):689/691 [M + H]⁺ |
| 4 | 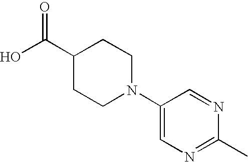 | 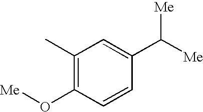 | MS (m/z):738 [M + H]⁺ |
| 5 | 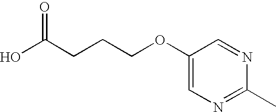 | 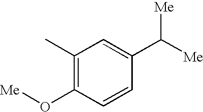 | MS (m/z):713 [M + H]⁺ |
| 6 | 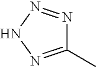 | 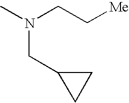 | MS (m/z):564 [M + H]⁺ |
| 7 | 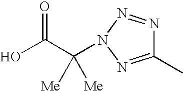 | 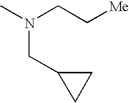 | MS (m/z):650 [M + H]⁺ |
| 8 | 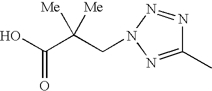 | 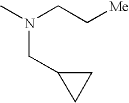 | MS (m/z):664 [M + H]⁺ |

### Example 9

(1) 2-Chloro-6-methoxy-quinoline-3-carboxaldehyde (2.0 g) and cyclopropylmethyl-propyl-amine (1.53 g) are dissolved in toluene (10 ml), and thereto is potassium carbonate (3.74 g) and the mixture is stirred at 120° C. overnight. The reaction solution is cooled to room temperature, and thereto is added water and the mixture is separated, and the organic layer is washed with a saturated brine, dried over magnesium sulfate, and concentrated under reduced pressure. The resulting residue is purified by silica gel column chromatography (hexane:ethyl acetate=99:1→9:1) to give 2-(cyclopropylmethyl-propyl-amino)-6-methoxy-quinoline-3-carboxaldehyde (2.03 g). MS (m/z): 299 [M+H]⁺

(2) 2-(Cyclopropylmethyl-propyl-amino)-6-methoxy-quinoline-3-carboxaldehyde (1.62 g) is dissolved in ethanol (10 ml) and thereto is added sodium borohydride (205 mg) at room temperature, and the mixture is stirred at room temperature for 1 hour. To the reaction solution are added a saturated aqueous ammonium chloride solution and diethyl ether, and the mixture is separated, and the organic layer is washed with a saturated brine, dried over magnesium sulfate, and concentrated under reduced pressure. The resulting residue is purified by silica gel column chromatography (hexane:ethyl acetate=9:1→7:3) to give [2-(cyclopropylmethyl-propyl-amino)-6-methoxy-quinolin-3-yl]-methanol (1.64 g). MS (m/z): 301 [M+H]⁺

(3) [2-(Cyclopropylmethyl-propyl-amino)-6-methoxy-quinolin-3-yl]-methanol (1.54 g) is dissolved in methylene chloride (5 ml), and thereto is added thionyl chloride (411 μl) under ice-cooling, and the mixture is stirred at the same temperature for 15 minutes and concentrated under reduced pressure. The resulting residue and (3,5-bis-trifluoromethyl-benzyl)-(5-bromopyrimidin-2-yl)-amine (2.05 g) are dissolved in N,N-dimethylformamide (10 ml), and thereto is added sodium hydride (62%) (397 mg) under ice-cooling, and the mixture is stirred at room temperature overnight. To the reaction solution are added diethyl ether and water, and the mixture is separated, and the organic layer is washed with a saturated brine, dried over magnesium sulfate, and concentrated under reduced pressure. The resulting residue is purified by silica gel column chromatography (hexane:ethyl acetate=99:1→9:1) to give (3-{[(3,5-bis-trifluoromethyl-benzyl)-(5-bromo-pyrimidin-2-yl)-amino]-methyl}-6-methoxy-quinolin-2-yl)-cyclopropylmethyl-propyl-amine (2.94 g). MS (m/z): 682/684 [M+H]⁺

### Example 10

The corresponding starting compound is treated in a similar manner to Example 4 to give the compound as listed in Table 2.

TABLE 2

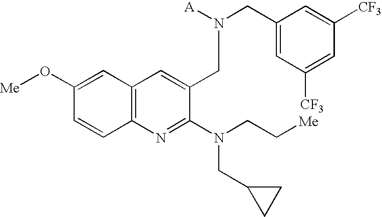

| Ex. | | Physical |
|---|---|---|
| No. | A- | properties, etc. |
| 9 | 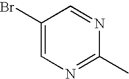 | MS (m/z):682/684 [M + H]⁺ |
| 10 | 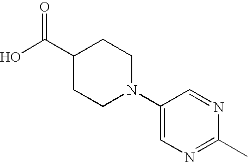 | MS (m/z):731 [M + H]⁺ |

### Example 11

The corresponding starting compound is treated in a similar manner to Example 9 to give the compound as listed in Table 3.

### Example 12

The corresponding starting compound is treated in a similar manner to Example 4 to give the compound as listed in Table 3.

TABLE 3

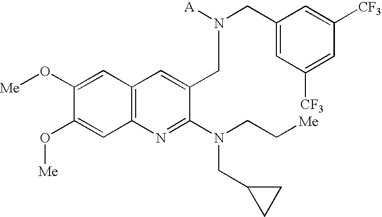

| Ex. | | Physical |
|---|---|---|
| No. | A- | properties, etc. |
| 11 | 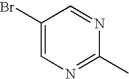 | MS (m/z):712/714 [M + H]⁺ |
| 12 | 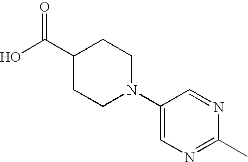 | MS (m/z):761 [M + H]⁺ |

### Example 13

The corresponding starting compound is treated in a similar manner to Example 9 to give the compound as listed in Table 4.

### Example 14

The corresponding starting compound is treated in a similar manner to Example 4 to give the compound as listed in Table 4.

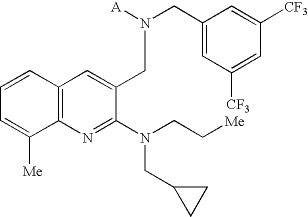

| Ex. | | Physical |
|---|---|---|
| No. | A- | properties, etc. |
| 13 | 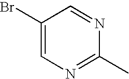 | MS (m/z):666/668 [M + H]⁺ |
| 14 | 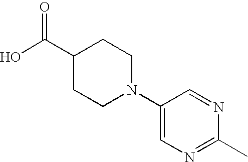 | MS (m/z):715 [M + H]⁺ |

### Example 15

The corresponding starting compound is treated in a similar manner to Example 6 to give the compound as listed in Table 5.

### Example 16

The corresponding starting compound is treated in a similar manner to Example 7 to give the compound as listed in Table 5.

### Example 17

The corresponding starting compound is treated in a similar manner to Example 8 to give the compound as listed in Table 5.

### Example 18

The corresponding starting compound is treated in a similar manner to Example 9 to give the compound as listed in Table 5.

### Example 19

The corresponding starting compound is treated in a similar manner to Example 4 to give the compound as listed in Table 5.

### Example 20

(1) (3,5-Bis-trifluoromethyl-benzyl)-(5-bromo-pyrimidin-2-yl)-[5-(cyclopropylmethyl-propyl-amino)-1-methyl-3-trifluoromethyl-1H-pyrazol-4-ylmethyl]-amine (500 mg) is dissolved in 1,4-dioxane (5 ml), and thereto are added benzyl acrylate (217 mg), tri-tert-butylphosphine tetrafluoroboric acid salt (21.5 mg), N-ethyldiisopropylamine (155 μl) and tris(dibenzylideneacetone)dipalladium (34 mg), and the mixture is stirred at room temperature under nitrogen flow overnight. To the reaction solution are added diethyl ether and water, and the mixture is separated. The organic layer is washed with a saturated brine, dried over magnesium sulfate, and concentrated under reduced pressure. The resulting residue is purified by silica gel column chromatography (hexane:ethyl acetate=91:9→4:1) to give benzyl 3-(2-{(3,5-bis-trifluoromethyl-benzyl)-[5-(cyclopropylmethyl-propyl-amino)-1-methyl-3-trifluoromethyl-1H-pyrazol-4-ylmethyl]-amino}-pyrimidin-5-yl)-acrylate (447 mg). MS (m/z): 755 [M+H]⁺

(2) Benzyl 3-(2-{(3,5-bis-trifluoromethyl-benzyl)-[5-(cyclopropylmethyl-propyl-amino)-1-methyl-3-trifluoromethyl-1H-pyrazol-4-ylmethyl]-amino}-pyrimidin-5-yl)-acrylate (440 mg) is dissolved in a mixed solvent of tetrahydrofuran (5 ml) and ethanol (1 ml), and thereto is added 10% palladium-carbon (50 mg) and the mixture is stirred under hydrogen atmosphere at room temperature overnight. The catalyst is removed by filtration, and the filtrate is concentrated under reduced pressure. The resulting residue is purified by silica gel column chromatography (chloroform:methanol=1:0→47:3) to give 3-(2-{(3,5-bis-trifluoromethyl-benzyl)-[5-(cyclopropylmethyl-propyl-amino)-1-methyl-3-trifluoromethyl-1H-pyrazol-4-ylmethyl]-amino}-pyrimidin-5-yl)-propionic acid (363 mg). MS (m/z): 667 [M+H]⁺

TABLE 5

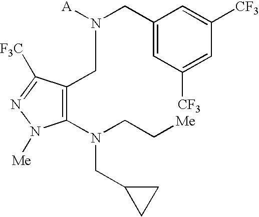

| Ex. | | Physical |
|---|---|---|
| No. | A- | properties, etc. |
| 15 | 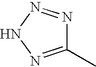 | MS (m/z):585 [M + H]⁺ |
| 16 | 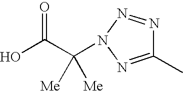 | MS (m/z):671 [M + H]⁺ |
| 17 | 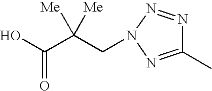 | MS (m/z):685 [M + H]⁺ |
| 18 | 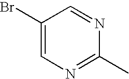 | MS (m/z):673/675 [M + H]⁺ |
| 19 | 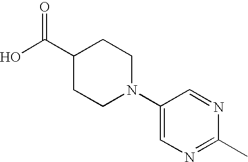 | MS (m/z):722 [M + H]⁺ |
| 20 | 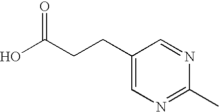 | MS (m/z):667 [M + H]⁺ |

### Example 21

(1) 5-Chloro-2-fluoropyridine-3-carboxaldehyde (970 mg) and cyclopropylmethyl-propyl-amine (1.3 ml) are dissolved in toluene (10 ml), and thereto is added potassium carbonate (2.52 g) and the mixture is stirred at 120° C. for 30 minutes. The reaction solution is cooled to room temperature, and thereto are added ethyl acetate and water, and the mixture is separated, and the organic layer is washed with a saturated brine, dried over magnesium sulfate, and concentrated under reduced pressure. The resulting residue is purified by silica gel column chromatography (hexane:ethyl acetate=19:1→17:3) to give 5-chloro-2-(cyclopropylmethyl-propyl-amino)-pyridine-3-carboxaldehyde (1.49 g). MS (m/z): 253/255 [M+H]⁺

(2) 5-Chloro-2-(cyclopropylmethyl-propyl-amino)-pyridine-3-carboxaldehyde (1.45 g) is dissolved in ethanol (10 ml) and thereto is added sodium borohydride (217 mg) at room temperature and the mixture is stirred for 1 hour. To the reaction solution are added a saturated aqueous ammonium chloride solution and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=24:1→17:3) to give [5-chloro-2-(cyclopropylmethyl-propyl-amino)-pyridin-3-yl]-methanol (1.35 g). MS (m/z): 255/257 [M+H]⁺

(3) [5-Chloro-2-(cyclopropylmethyl-propyl-amino)-pyridin-3-yl]-methanol obtained in the above (2) as the starting compound is treated in similar manners to Examples 1(2) and (3) to give 4-(2-{(3,5-bis-trifluoromethyl-benzyl)-[5-chloro-2-(cyclopropylmethyl-propyl-amino)-pyridin-3-ylmethyl]-amino}-pyrimidin-5-yloxy)-butyric acid sodium salt. MS (m/z): 658/660 [M-Na]

### Example 22

[5-Chloro-2-(cyclopropylmethyl-propyl-amino)-pyridin-3-yl]-methanol prepared in the Example 21(2) as the starting compound is treated in a similar manner to Example 9(3) to give (3,5-bis-trifluoromethyl-benzyl)-(5-bromo-pyrimidin-2-yl)-[5-chloro-2-(cyclopropylmethyl-propyl-amino)-pyridin-3-ylmethyl]-amine. MS (m/z): 636/638 [M+H]⁺

### Example 23

The corresponding starting compound is treated in a similar manner to Example 4 to give the compound as listed in Table 6.

### Example 24

Thereto are added (3,5-bis-trifluoromethyl-benzyl)-(5-bromo-pyrimidin-2-yl)-[5-chloro-2-(cyclopropylmethyl-propyl-amino)-pyridin-3-ylmethyl]-amine (150 mg) is dissolved in toluene (5 ml) and thereto are added tris(dibenzylideneacetone)dipalladium (22 mg), sodium tert-butoxide (34 mg) and 2-(di-tert-butylphosphino)biphenyl (28 mg) and morpholine (31 μl), and the mixture is stirred under nitrogen flow at room temperature overnight. The reaction solution is washed with a saturated brine, and the organic layer is dried over magnesium sulfate and concentrated under reduced pressure. The resulting residue is purified by NH-silica gel column chromatography (hexane:ethyl acetate=49:1→87:13) to give (3,5-bis-trifluoromethyl-benzyl)-[5-chloro-2-(cyclopropylmethyl-propyl-amino)-pyridin-3-ylmethyl]-(5-morpholin-4-yl-pyrimidin-2-yl)-amine (88 mg). MS (m/z): 643/645 [M+H]⁺

TABLE 6

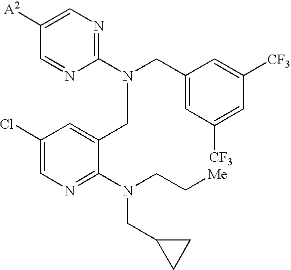

| Ex. | | Physical |
|---|---|---|
| No. | A²- | properties, etc. |
| 21 | 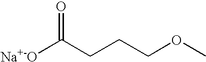 | MS (m/z):658/660 [M − Na]⁻ |
| 22 | 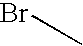 | MS (m/z):636/638 [M + H]⁺ |
| 23 | 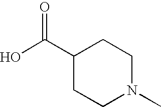 | MS (m/z):685/687 [M + H]⁺ |
| 24 | 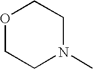 | MS (m/z):643/645 [M + H]⁺ |

### Example 25

(1) Ethyl 4-chloro-2-methylsulfanyl-pyrimidine-5-carboxylate (10.00 g) is dissolved in tetrahydrofuran (120 ml) and thereto are added cyclopropylmethyl-propyl-amine (7.38 ml) and triethylamine (7.19 ml) in water bath, and the mixture is stirred at room temperature for 1 hour. To the reaction mixture is added a saturated brine, and the mixture is extracted with ethyl acetate and the organic layer is washed with a saturated brine, dried over magnesium sulfate, and concentrated under reduced pressure. The resulting residue is purified by silica gel column chromatography (hexane:ethyl acetate=17:3) to give ethyl 4-(cyclopropylmethyl-propyl-amino)-2-methylsulfanyl-pyrimidine-5-carboxyate (11.07 g). MS (m/z): 310 [M+H]⁺

(2) Ethyl 4-(cyclopropylmethyl-propyl-amino)-2-methylsulfanyl-pyrimidine-5-carboxylate (10.58 g) is dissolved in tetrahydrofuran (50 ml), and to a suspension of lithium aluminum hydride (1.30 g) and tetrahydrofuran (50 ml) is added dropwise the resulting solution under ice-cooling, and the mixture is stirred at the same temperature for 2 hours. To the reaction mixture are added a 1M aqueous sodium hydroxide solution (1.3 ml) and water (1.3 ml) under ice-cooling, and the mixture is stirred at the same temperature for 30 minutes, and the resulting precipitate is filtered and the resulting filtrate is dried over magnesium sulfate and concentrated under reduced pressure. The resulting residue is purified by NH-silica gel column chromatography (hexane:ethyl acetate=2:1→1:4) to give 4-(cyclopropylmethyl-propyl-amino)-2-methylsulfanyl-pyrimidin-5-yl]-methanol (2.24 g). MS (m/z): 268 [M+H]⁺

(3) [4-(Cyclopropylmethyl-propyl-amino)-2-methylsulfanyl-pyrimidin-5-yl]-methanol (1.49 g) is dissolved in chloroform (25 ml) and thereto is added manganese dioxide (5 g), and the mixture is stirred at room temperature overnight. The manganese dioxide is removed by filtration and the resulting filtrate is concentrated under reduced pressure, and to the resulting residue are added hexane and isopropylether, and the mixture is filtered to give 4-(cyclopropylmethyl-propyl-amino)-2-methylsulfanyl-pyrimidine-5-carbaldehyde (1.27 g). MS (m/z): 266 [M+H]⁺

(4) 4-(Cyclopropylmethyl-propyl-amino)-2-methylsulfanyl-pyrimidine-5-carbaldehyde (1.27 g) is dissolved in 1,2-dichloroethane (15 ml), and thereto are added 3,5-bis-(trifluoromethyl)benzylamine (1.51 g), acetic acid (0.55 ml) and triacetoxy-sodium borohydride (2.03 g) and the mixture is stirred at room temperature for 1 hour. To the reaction mixture is added a saturated aqueous sodium bicarbonate solution and the mixture is extracted with ethyl acetate. The organic layer is washed twice with a saturated brine, dried over magnesium sulfate, and concentrated under reduced pressure. The resulting residue is purified by silica gel column chromatography (hexane:ethyl acetate=4:1) to give {5-[(3,5-bis-trifluoromethyl-benzylamino)-methyl]-2-methylsulfanyl-pyrimidin-4-yl}-cyclopropylmethyl-propyl-amine (2.06 g). MS (m/z): 493 [M+H]⁺

(5) {5-[(3,5-Bis-trifluoromethyl-benzylamino)-methyl]-2-methylsulfanyl-pyrimidin-4-yl}-cyclopropylmethyl-propyl-amine (2.05 g) is dissolved in toluene (15 ml), and thereto are added 5-bromo-2-chloropyrimidine (3.22 g) and N,N-diisopropylethylamine (4.34 ml) and the mixture is stirred at 110° C. under nitrogen atmosphere for 3 days. To the reaction mixture is added a saturated brine, and the mixture is extracted with ethyl acetate and the organic layer is washed with a saturated brine, dried over magnesium sulfate, and concentrated under reduced pressure. To the resulting residue are added hexane and isopropylether, and the precipitated insoluble material is filtered and the filtrate is concentrated under reduced pressure, and the resulting residue is purified by silica gel column chromatography (hexane:ethyl acetate=9:1→4:1) to give (5-{[(3,5-bis-trifluoromethyl-benzyl)-(5-bromo-pyrimidin-2-yl)-amino]-methyl}-2-methylsulfanyl-pyrimidin-4-yl)-cyclopropylmethyl-propyl-amine (2.13 g). MS (m/z): 651/649 [M+H]⁺

(6) (5-{[(3,5-Bis-trifluoromethyl-benzyl)-(5-bromo-pyrimidin-2-yl)-amino]-methyl}-2-methylsulfanyl-pyrimidin-4-yl)-cyclopropylmethyl-propyl-amine (1.00 g) is dissolved in chloroform (7 ml) and thereto is added m-chloroperbenzoic acid (1.10 g) and the mixture is stirred at room temperature for 3 hours. To the reaction solution is added a saturated aqueous sodium bicarbonate solution, and the mixture is extracted with chloroform and the organic layer is washed successively with a saturated aqueous sodium bicarbonate solution and brine, dried over magnesium sulfate, and concentrated under reduced pressure. The resulting residue is purified by silica gel column chromatography (hexane:ethyl acetate=4:1→3:1) to give (5-{[(3,5-bis-trifluoromethyl-benzyl)-(5-bromo-pyrimidin-2-yl)-amino]-methyl}-2-methane sulfonyl-pyrimidin-4-yl)-cyclopropylmethyl-propyl-amine (858 mg). MS (m/z): 683/681 [M+H]⁺

(7) (5-{[(3,5-Bis-trifluoromethyl-benzyl)-(5-bromo-pyrimidin-2-yl)-amino]-methyl}-2-methanesulfonyl-pyrimidin-4-yl)-cyclopropylmethyl-propyl-amine (500 mg) is dissolved in methanol (5 ml), and thereto is added a 28% sodium methoxide in methanol (231 μl) and the mixture is stirred at room temperature overnight. The reaction mixture solution is concentrated under reduced pressure and thereto is added a 10% aqueous citric acid solution, and the mixture is extracted with ethyl acetate and the organic layer is washed successively with a saturated aqueous sodium bicarbonate solution and a saturated brine, dried over magnesium sulfate, and concentrated under reduced pressure. The resulting residue is purified by silica gel column chromatography (hexane:ethyl acetate=17:3→7:3) to give (5-{[(3,5-bis-trifluoromethyl-benzyl)-(5-bromo-pyrimidin-2-yl)-amino]-methyl}-2-methoxy-pyrimidin-4-yl)-cyclopropylmethyl-propyl-amine (425 mg). MS (m/z): 635/633 [M+H]⁺

(8) (5-{[(3,5-Bis-trifluoromethyl-benzyl)-(5-bromo-pyrimidin-2-yl)-amino]-methyl}-2-methoxy-pyrimidin-4-yl)-cyclopropylmethyl-propyl-amine (422 mg) is dissolved in a mixed solvent of dimethylsulfoxide (2 ml) and methylene chloride (0.5 ml), and the mixture is degassed, and thereto are added [1,1′-bis(diphenylphosphino)ferrocene]palladium(II) dichloride (27 mg), potassium acetate (196 mg) and bis(pinacolato)diboron (254 mg), and the mixture is heated to 80° C. under nitrogen flow and stirred for 45 minutes. The reaction solution is allowed to cool to room temperature and thereto is added a saturated brine, and the mixture is extracted with ethyl acetate, and the organic layer is washed twice with a saturated brine, dried over magnesium sulfate, and concentrated under reduced pressure. The resulting residue is dissolved in tetrahydrofuran (2.5 ml), and thereto is added dropwise a 30% aqueous hydrogen peroxide solution (0.75 ml) under ice-cooling and the mixture is stirred at the same temperature for 1 hour. To the reaction mixture is added dropwise a saturated aqueous sodium thiosulfate solution under ice-cooling and the mixture is extracted with ethyl acetate. The organic layer is washed with a saturated brine, dried over magnesium sulfate, and concentrated under reduced pressure. To the resulting residue is added isopropylether, and the precipitated solid is filtered to give 2-{(3,5-bis-trifluoromethyl-benzyl)-[4-(cyclopropylmethyl-propyl-amino)-2-methoxy-pyrimidin-5-ylmethyl]-amino}-pyrimidin-5-ol (328 mg). MS (m/z): 571 [M+H]⁺

(9) 2-{(3,5-Bis-trifluoromethyl-benzyl)-[4-(cyclopropylmethyl-propyl-amino)-2-methoxy-pyrimidin-5-ylmethyl]-amino}-pyrimidin-5-ol (181 mg) is dissolved in N,N-dimethylformamide (2 ml), and thereto are added potassium carbonate (132 mg) and ethyl 4-bromo-butyrate (160 μl), and the mixture is stirred at room temperature overnight. To the reaction mixture is added a saturated brine, and the mixture is extracted with ethyl acetate, and the organic layer is washed with a saturated brine, dried over magnesium sulfate, and concentrated under reduced pressure. The resulting residue is purified by silica gel column chromatography (hexane:ethyl acetate=4:1→13:7) to give ethyl 4-(2-{(3,5-bis-trifluoromethyl-benzyl)-[4-(cyclopropylmethyl-propyl-amino)-2-methoxy-pyrimidin-5-ylmethyl]-amino}-pyrimidin-5-yloxy)-butyrate (152 mg). MS (m/z): 685 [M+H]⁺

(10) Ethyl 4-(2-{(3,5-bis-trifluoromethyl-benzyl)-[4-(cyclopropylmethyl-propyl-amino)-2-methoxy-pyrimidin-5-ylmethyl]-amino}-pyrimidin-5-yloxy)-butyrate (148 mg) is dissolved in a mixed solvent of ethanol (1 ml) and tetrahydrofuran (0.5 ml), and thereto is added a 1M-aqueous sodium hydroxide solution (1 ml), and the mixture is stirred at room temperature for 2 hours. To the reaction mixture is added a 10% aqueous citric acid solution and a saturated brine, and the mixture is extracted with ethyl acetate and the organic layer is washed with a saturated brine, dried over magnesium sulfate, and concentrated under reduced pressure. To the resulting residue is added ether and the precipitated solid is filtered to give 4-(2-{(3,5-bis-trifluoromethyl-benzyl)-[4-(cyclopropylmethyl-propyl-amino)-2-methoxy-pyrimidin-5-ylmethyl]-amino}-pyrimidin-5-yloxy)-butyric acid (122 mg). MS (m/z): 657 [M+H]⁺

### Example 26

(1) (5-{[(3,5-Bis-trifluoromethyl-benzyl)-(5-bromo-pyrimidin-2-yl)-amino]-methyl}-2-methanesulfonyl-pyrimidin-4-yl)-cyclopropylmethyl-propyl-amine (450 mg) is dissolved in ether (3 ml) and thereto is added dropwise a 3M methylmagnesium chloride in tetrahydrofuran (286 μl) under ice-cooling, and the mixture is stirred at the same temperature for 1 hour and a half. To the reaction mixture solution are added a saturated brine and ethyl acetate, and the insoluble materials are removed by filtration through Celite™, and the organic layer is washed with a saturated brine, dried over magnesium sulfate, and concentrated under reduced pressure. The resulting residue is purified by silica gel column chromatography (hexane:ethyl acetate=4:1→3:1) to give (5-{[(3,5-bis-trifluoromethyl-benzyl)-(5-bromo-pyrimidin-2-yl)-amino]-methyl}-2-methyl-pyrimidin-4-yl)-cyclopropylmethyl-propyl-amine (346 mg). MS (m/z): 619/617 [M+H]⁺

(2) (5-{[(3,5-Bis-trifluoromethyl-benzyl)-(5-bromo-pyrimidin-2-yl)-amino]-methyl}-2-methyl-pyrimidin-4-yl)-cyclopropylmethyl-propyl-amine is treated in similar manners to Examples 25(8)-(10) to give 4-(2-{(3,5-bis-trifluoromethyl-benzyl)-[4-(cyclopropylmethyl-propyl-amino)-2-methyl-pyrimidin-5-ylmethyl]-amino}-pyrimidin-5-yloxy)-butyric acid. MS (m/z): 641 [M+H]⁺

TABLE 7

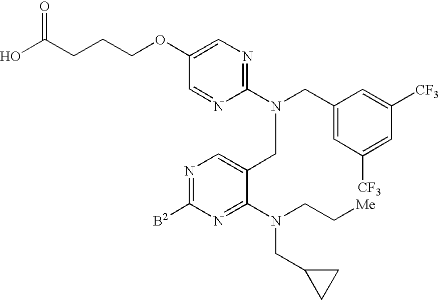

| Ex. | | |
|---|---|---|
| No. | B²— | Physical properties, etc. |
| 25 | 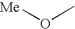 | MS (m/z):657 [M + H]⁺ |
| 26 | 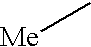 | MS (m/z):641 [M + H]⁺ |

### Example 27

(6-Bromo-pyridin-2-yl)-methanol (1.0 g) is dissolved in methylene chloride (10 ml) and thereto is added thionyl chloride (427 μl) under ice-cooling, and the mixture is stirred at the same temperature for 15 minutes. To the reaction solution is added a saturated aqueous sodium bicarbonate solution, and the mixture is separated, and the organic layer is washed with a saturated brine, dried over magnesium sulfate, and concentrated under reduced pressure. The resulting residue and (3,5-bis-trifluoromethyl-benzyl)-5-bromopyrimidin-2-yl)-amine (2.12 g) is dissolved in N,N-dimethylformamide (20 ml), and thereto is added sodium hydride (62%) (226 mg) under ice-cooling, and mixture is stirred at room temperature overnight. To the reaction solution are added diethyl ether and water, and the mixture is separated, and the organic layer is washed with a saturated brine, dried over magnesium sulfate, and concentrated under reduced pressure. The resulting residue is purified by silica gel column chromatography (hexane:ethyl acetate=1:0→19:1) to give (3,5-bis-trifluoromethyl-benzyl)-(5-bromo-pyrimidin-2-yl)-(6-bromo-pyridin-2-ylmethyl)-amine (2.7 g). MS (m/z): 569/571 [M+H]⁺

TABLE 8

| Ex. | | Physical |
|---|---|---|
| No. | [symbol] | properties, etc. |
| 27 | 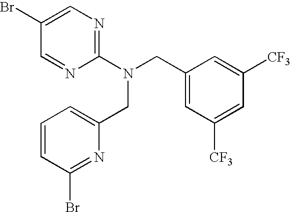 | MS (m/z):569/571[M + H]⁺ |

### Example 28

(1) Tert-butyl 7-formyl-2,3-dihydro-indole-1-carboxylate (2.0 g) is dissolved in a mixed solvent of toluene (5 ml) and ethanol (1 ml), and thereto is added sodium borohydride (367 mg), and the mixture is stirred at room temperature for 2 hours. To the reaction solution are added a saturated aqueous ammonium chloride solution and ethyl acetate, and the mixture is separated, and the organic layer is washed with a saturated brine, dried over magnesium sulfate, and concentrated under reduced pressure. The resulting residue is purified by silica gel column chromatography (hexane ethyl acetate=9:1→83:17) to give tert-butyl 7-hydroxymethyl-2,3-dihydro-indole-1-carboxylate (1.79 g).

(2) Tert-butyl 7-hydroxymethyl-2,3-dihydro-indole-1-carboxylate (1.79 g) is dissolved in toluene (30 ml) and thereto is added thionyl chloride (630 μl) under ice-cooling, and the mixture is stirred at room temperature for 1 hour. To the reaction solution are added water and ethyl acetate, and the mixture is separated, and the organic layer is washed with a saturated brine, dried over magnesium sulfate, and concentrated under reduced pressure. The resulting residue is dissolved in N,N-dimethylformamide (30 ml) and thereto are added (3,5-bis-trifluoromethyl-benzyl)-(5-bromo-pyrimidin-2-yl)-amine (3.4 g) and sodium tert-butoxide (830 mg), and the mixture is stirred at room temperature for 3 days. To the reaction solution are added water and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=1:0→17:3) to give tert-butyl 7-{[(3,5-bis-trifluoromethyl-benzyl)-(5-bromo-pyrimidin-2-yl)-amino]-methyl}-2,3-dihydro-indole-1-carboxylate (3.5 g). MS (m/z): 631/633 [M+H]⁺

(3) Tert-butyl 7-{[(3,5-bis-trifluoromethyl-benzyl)-(5-bromo-pyrimidin-2-yl)-amino]-methyl}-2,3-dihydro-indole-1-carboxylate (2.0 g) is dissolved in toluene (30 ml), and thereto are added tris(dibenzylideneacetone)-dipalladium (290 mg), 2-(di-tert-butylphosphino)biphenyl (380 mg), sodium tert-butoxide (457 mg) and morpholine (415 μl), and the mixture is stirred at room temperature under nitrogen atmosphere overnight, and to the reaction solution are added tris(dibenzylideneacetone)dipalladium (290 mg), 2-(di-tert-butylphosphino)biphenyl (380 mg) and sodium tert-butoxide (457 mg), and the mixture is stirred for 3 hours. To the reaction solution are added a saturated aqueous sodium bicarbonate solution and ethyl acetate, and the mixture is separated, and the organic layer is washed with a saturated brine, dried over magnesium sulfate, and concentrated under reduced pressure. The resulting residue is purified by NH-silica gel column chromatography (hexane:ethyl acetate=9:1→7:3) to give tert-butyl 7-{[(3,5-bis-trifluoromethyl-benzyl)-(5-morpholin-4-yl-pyrimidin-2-yl)-amino]-methyl}-2,3-dihydro-indole-1-carboxylate (1.53 g). MS (m/z): 638 [M+H]⁺

(4) Tert-butyl 7-{[(3,5-bis-trifluoromethyl-benzyl)-(5-morpholin-4-yl-pyrimidin-2-yl)-amino]-methyl}-2,3-dihydro-indole-1-carboxylate (1.53 g) is dissolved in a 4N-hydrochloric acid in dioxane (5 ml), and the mixture is stirred at room temperature for 1 hour and 30 minutes. To the reaction solution are added a saturated aqueous sodium bicarbonate solution and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=4:1→3:2) to give (3,5-bis-trifluoromethyl-benzyl)-(2,3-dihydro-1H-indol-7-ylmethyl)-(5-morpholin-4-yl-pyrimidin-2-yl)-amine (1.15 g). MS (m/z): 538 [M+H]⁺

### Example 29

(1) (3,5-Bis-trifluoromethyl-benzyl)-(2,3-dihydro-1H-indol-7-ylmethyl)-(5-morpholin-4-yl-pyrimidin-2-yl)-amine (450 mg) is dissolved in N,N-dimethylformamide (10 ml), and thereto is added sodium hydride (60%) (47 mg) under ice-cooling and the mixture is stirred for 30 minutes, and thereto is added ethyl 7-bromo-heptanoate (275 mg) and the mixture is stirred at room temperature overnight. To the reaction solution are added water and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=4:1→3:2) to give ethyl 7-(7-{[(3,5-bis-trifluoromethyl-benzyl)-(5-morpholin-4-yl-pyrimidin-2-yl)-amino]-methyl}-indol-1-yl)-heptanoate (95 mg). MS (m/z): 692 [M+H]⁺

(2) Ethyl 7-(7-{[(3,5-bis-trifluoromethyl-benzyl)-(5-morpholin-4-yl-pyrimidin-2-yl)-amino]-methyl}-indol-1-yl)-heptanoate (90 mg) is dissolved in ethanol (4 ml), and thereto is added a 1N-aqueous sodium hydroxide solution (1 ml) and the mixture is stirred at room temperature overnight. To the reaction solution are added a 1N-hydrochloric acid and ethyl acetate, and the mixture is separated, and the organic layer is washed with a saturated brine, dried over magnesium sulfate, and concentrated under reduced pressure. The resulting residue is purified by silica gel column chromatography (chloroform:methanol=1:0→9:1) to give 7-(7-{[(3,5-bis-trifluoromethyl-benzyl)-(5-morpholin-4-yl-pyrimidin-2-yl)-amino]-methyl}-indol-1-yl)-heptanoic acid (76 mg). MS (m/z): 664 [M+H]⁺

### Example 30

(1) (3,5-Bis-trifluoromethyl-benzyl)-(2,3-dihydro-1H-indol-7-ylmethyl)-(5-morpholin-4-yl-pyrimidin-2-yl)-amine (150 mg) is dissolved in methylene chloride (5 ml), and thereto are added ethyl 6-(chloroformyl)hexanoate (86 mg) and triethylamine (34 μl) under ice-cooling, and the mixture is stirred at room temperature for 2 hours. To the reaction solution are added a saturated aqueous ammonium chloride solution and chloroform, and the mixture is separated, and the organic layer is washed with a saturated brine, dried over magnesium sulfate, and concentrated under reduced pressure. The resulting residue is purified by silica gel column chromatography (hexane:ethyl acetate=3:2→9:11) to give ethyl 7-(7-{[(3,5-bis-trifluoromethyl-benzyl)-(5-morpholin-4-yl-pyrimidin-2-yl)-amino]-methyl}-2,3-dihydro-indol-1-yl)-7-oxo-heptanoate (135 mg). MS (m/z): 708 [M+H]⁺

(2) Ethyl 7-(7-{[(3,5-bis-trifluoromethyl-benzyl)-(5-morpholin-4-yl-pyrimidin-2-yl)-amino]-methyl}-2,3-dihydro-indol-1-yl)-7-oxo-heptanoate (130 mg) is dissolved in ethanol (4 ml), and thereto is added a 1N-aqueous sodium hydroxide solution (1 ml), and the mixture is stirred at room temperature for 3 hours. To the reaction solution are added a 1N-hydrochloric acid and ethyl acetate, and the mixture is separated, and the organic layer is washed with a saturated brine, dried over magnesium sulfate, and concentrated under reduced pressure. The resulting residue is purified by silica gel column chromatography (chloroform:methanol=1:0→9:1) to give 7-(7-{[(3,5-bis-trifluoromethyl-benzyl)-(5-morpholin-4-yl-pyrimidin-2-yl)-amino]-methyl}-2,3-dihydro-indol-1-yl)-7-oxo-heptanoic acid (116 mg). MS (m/z): 680 [M+H]⁺

### Example 31

(1) (3,5-Bis-trifluoromethyl-benzyl)-(2,3-dihydro-1H-indol-7-ylmethyl)-(5-morpholin-4-yl-pyrimidin-2-yl)-amine (250 mg) and triethylamine (77 μl) are dissolved in methylene chloride (8 ml), and thereto is added triphosgene (55 mg) under ice-cooling, and the mixture is stirred at room temperature under nitrogen atmosphere for 1 hour. To the reaction solution are added a saturated aqueous sodium bicarbonate solution and chloroform, and the mixture is separated, and the organic layer is washed with a saturated brine, dried over magnesium sulfate, and concentrated under reduced pressure. The resulting residue is dissolved in tetrahydrofuran (5 ml), and thereto are added methyl 3-hydroxy-2,2-dimethyl-propionate (360 μl) and sodium hydride (60%) (108 mg) and the mixture is stirred at room temperature overnight. To the reaction solution are added water and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=4:113:2) to give 7-{[(3,5-bis-trifluoromethyl-benzyl)-(5-morpholin-4-yl-pyrimidin-2-yl)-amino]-methyl}-2,3-dihydro-indole-1-carboxylic acid 2-methoxycarbonyl-2-methyl-propylester (86 mg). MS (m/z): 696 [M+H]⁺

(2) 7-{[(3,5-Bis-trifluoromethyl-benzyl)-(5-morpholin-4-yl-pyrimidin-2-yl)-amino]-methyl}-2,3-dihydro-indole-1-carboxylic acid 2-methoxycarbonyl-2-methyl-propylester (80 mg) is dissolved in ethanol (4 ml), and thereto is added a 1N-aqueous sodium hydroxide solution (1 ml), and the mixture is stirred at room temperature for 4 hours and 30 minutes. To the reaction solution are added a 1N-hydrochloric acid and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=3:2→2:3) to give 7-{[(3,5-bis-trifluoromethyl-benzyl)-(5-morpholin-4-yl-pyrimidin-2-yl)-amino]-methyl}-2,3-dihydro-indole-1-carboxylic acid 2-carboxy-2-methylpropylester (25 mg). MS (m/z): 682 [M+H]⁺

### Example 32

The corresponding starting compound is treated in a similar manner to Example 5 to give the compound as listed in Table 9.

TABLE 9

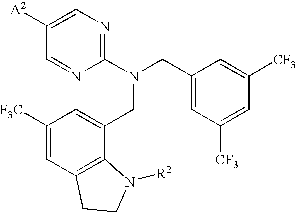

| Ex. | | | Physical |
|---|---|---|---|
| No. | A²- | —R² | properties, etc. |
| 28 | 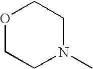 | 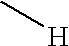 | MS (m/z):538 [M + H]⁺ |
| 29 | 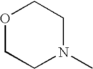 | 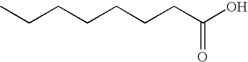 | MS (m/z):664 [M + H]⁺ |
| 30 | 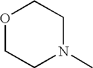 | 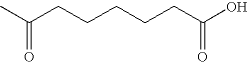 | MS (m/z):680 [M + H]⁺ |
| 31 | 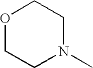 | 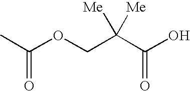 | MS (m/z):682 [M + H]⁺ |
| 32 | 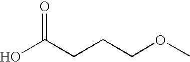 | 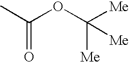 | MS (m/z):655 [M + H]⁺ |

### Example 33

1-Benzyl-2-chloromethyl-1H-imidazole hydrochloride (1.0 g) and (3,5-bis-trifluoromethyl-benzyl)-(5-bromopyrimidin-2-yl)-amine (1.65 g) are dissolved in N,N-dimethylformamide (20 ml), and thereto is added sodium hydride (60%) (318 mg) under ice-cooling, and the mixture is stirred at room temperature for 4 hours. To the reaction solution are added diethyl ether and water, and the mixture is separated, and the organic layer is washed with a saturated brine, dried over magnesium sulfate, and concentrated under reduced pressure. The resulting residue is purified by silica gel column chromatography (hexane:ethyl acetate=4:1→3:2) to give (1-benzyl-1H-imidazol-2-ylmethyl)-(3,5-bis-trifluoromethyl-benzyl)-(5-bromo-pyrimidin-2-yl)-amine (1.94 g). MS (m/z): 570/572 [M+H]⁺

### Example 34

The corresponding starting compound is treated in a similar manner to Example 24 to give the compound as listed in Table 10.

TABLE 10

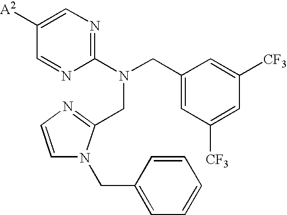

| Ex. | | |
|---|---|---|
| No. | A²- | Physical properties, etc. |
| 33 | 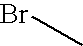 | MS (m/z):570/572 [M + H]⁺ |
| 34 | 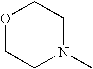 | MS (m/z):577 [M + H]⁺ |

### Example 35

(1) 2-Chloro-3-nitro-pyridine (3.17 g) and (aminomethyl)cyclopropane hydrochloride (2.15 g) are dissolved in N,N-dimethylformamide (10 ml), and thereto is added potassium carbonate (8.29 g) and the mixture is stirred at room temperature for 6 hours. The reaction solution is cooled to room temperature, and thereto are added ethyl acetate and water, and the mixture is separated, and the organic layer is washed with a saturated brine, dried over magnesium sulfate, and concentrated under reduced pressure. The resulting residue is dissolved in a mixed solvent of tetrahydrofuran (50 ml) and methanol (10 ml), and thereto is added Raney nickel, and the mixture is stirred at room temperature under hydrogen atmosphere overnight. The catalyst is removed by filtration, and the filtrate is concentrated under reduced pressure. The resulting residue is dissolved in chloroform, dried over magnesium sulfate, and concentrated under reduced pressure. The resulting residue is dissolved in ethanol (100 ml), and thereto is added 2-chloroacetimidic acid ethyl ester hydrochloride (31.6 g) and the mixture is stirred at 60° C. overnight. To the reaction solution are added ethyl acetate and a saturated aqueous sodium bicarbonate solution, and the mixture is separated, and the organic layer is washed with a saturated brine, dried over magnesium sulfate, and concentrated under reduced pressure. The resulting residue is purified by silica gel column chromatography (hexane:ethyl acetate=2:1→1:1) to give 2-chloromethyl-3-cyclopropylmethyl-3H-imidazo[4,5-b]pyridine (2.92 g).

(2) (3,5-Bis-trifluoromethyl-benzyl)-(5-bromo-pyrimidin-2-yl)-amine (361 mg) is dissolved in N,N-dimethylformamide (2 ml), and thereto is added sodium hydride (62%) (45 mg) under ice-cooling, and the mixture is stirred at the same temperature for 30 minutes. Then, to the reaction solution are added 2-chloromethyl-3-cyclopropylmethyl-3H-imidazo[4,5-b]pyridine (200 mg) and the mixture is stirred at room temperature for 15 minutes. To the reaction solution are added diethyl ether and water, and the mixture is separated, and the organic layer is washed with a saturated brine, dried over magnesium sulfate, and concentrated under reduced pressure. The resulting residue is purified by silica gel column chromatography (hexane ethyl acetate=9:1→19:6) to give (3,5-bis-trifluoromethyl-benzyl)-(5-bromo-pyrimidin-2-yl)-(1-cyclopropylmethyl-1H-imidazo[4,5-b]pyridin-2-ylmethyl)-amine (390 mg). MS (m/z): 585/587 [M+H]⁺

### Example 36

The corresponding starting compound is treated in a similar manner to Example 4 to give the compound as listed in Table 11.

TABLE 11

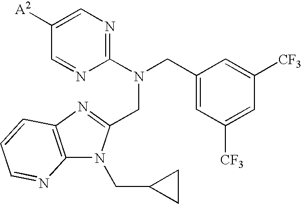

| Ex. | | Physical |
|---|---|---|
| No. | A²- | properties, etc. |
| 35 | 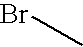 | MS (m/z):585/587 [M + H]⁺ |
| 36 | 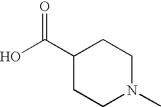 | MS (m/z):634 [M + H]⁺ |

### Example 37

Ethyl 4-chloro-2-methylsulfanyl-pyrimidine-5-carboxylate (200 mg) is dissolved in N,N-dimethylformamide (3 ml), and thereto are added 3,5-bis-(trifluoromethyl)benzylamine (313 mg) and triethylamine (360 μl), and the mixture is stirred at room temperature for 3 hours. To the reaction mixture is added water and the mixture is extracted with ethyl acetate, and the organic layer is washed with a saturated brine, dried over magnesium sulfate, and concentrated under reduced pressure. The resulting residue is purified by silica gel column chromatography (chloroform:hexane:ethyl acetate=10:20:1) to give ethyl 4-(3,5-bis-trifluoromethyl-benzylamino)-2-methylsulfanyl-pyrimidine-5-carboxylate (340 mg). MS (m/z): 440 [M+H]⁺

TABLE 12

| Ex. | | Physical |
|---|---|---|
| No. | Structural formula | properties, etc. |
| 37 | 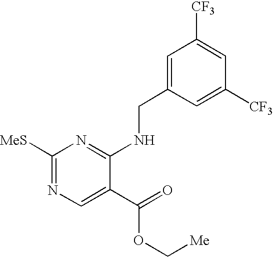 | as described above |

### Example 38

(1) To methanol (20 ml) is added dropwise thionyl chloride (1.21 ml) under ice-cooling, followed by an addition of 2-amino-3-(3,5-bis-trifluoromethyl-phenyl)-propionic acid (1.00 g), and the mixture is stirred at room temperature for 2 days. The reaction mixture is concentrated under reduced pressure, and to the resulting residue are added ether and hexane, and the precipitated crystals are filtered to give 2-amino-3-(3,5-bis-trifluoromethyl-phenyl)-propionic acid methyl ester hydrochloride (1.11 g). MS (m/z): 316 [M+H]⁺

(2) Ethyl 4-chloro-2-methylsulfanyl-pyrimidine-5-carboxylate (300 mg) is dissolved in N,N-dimethylformamide (4 ml), and thereto are added 2-amino-3-(3,5-bis-trifluoromethyl-phenyl)-propionic acid methyl ester hydrochloride (544 mg) and triethylamine (540 μl), and the mixture is stirred at room temperature for 3 hours. To the reaction mixture is added water and the mixture is extracted with ethyl acetate, and the organic layer is washed with water, dried over magnesium sulfate, and concentrated under reduced pressure. The resulting residue is purified by silica gel column chromatography (chloroform:hexane:ethyl acetate=10:12→:1) to give ethyl 4-[2-(3,5-bis-trifluoromethyl-phenyl)-1-methoxycarbonyl-ethylamino]-2-methylsulfanyl-pyrimidine-5-carboxylate (542 mg). MS (m/z): 512 [M+H]⁺

### Example 39

Ethyl 4-[2-(3,5-bis-trifluoromethyl-phenyl)-1-methoxycarbonyl-ethylamino]-2-methylsulfanyl-pyrimidine-5-carboxylate (490 mg) is dissolved in chloroform (6 ml) and thereto is added m-chloroperbenzoic acid (248 mg) under ice-cooling, and the mixture is stirred at the same temperature for 10 minutes. The reaction mixture solution is purified by silica gel column chromatography (chloroform:acetone=15:1→10:1) to give ethyl 4-[2-(3,5-bis-trifluoromethyl-phenyl)-1-methoxycarbonyl-ethylamino]-2-methanesulfinyl-pyrimidine-5-carboxylate (480 mg). MS (m/z): 528 [M+H]⁺

### Example 40

Ethyl 4-[2-(3,5-bis-trifluoromethyl-phenyl)-1-methoxycarbonyl-ethylamino]-2-methanesulfinyl-pyrimidine-5-carboxylate (236 mg) is dissolved in tetrahydrofuran (3 ml), and thereto is added five drops of a 28% sodium methoxide in methanol by pasteur pipette, and the mixture is stirred at room temperature for 5 minutes. The reaction mixture solution is neutralized with a 10% aqueous citric acid solution, and thereto is added a saturated aqueous sodium bicarbonate solution and the mixture is extracted with ethyl acetate, and the organic layer is dried over magnesium sulfate, and concentrated under reduced pressure. The resulting residue is purified by thin-layer silica gel column chromatography (hexane:ethyl acetate=4:1) to give ethyl 4-[2-(3,5-bis-trifluoromethyl-phenyl)-1-methoxy-carbonyl-ethylamino]-2-methoxy-pyrimidine-5-carboxylate (55 mg). MS (m/z): 496 [M+H]⁺

### Example 41

Ethyl 4-[2-(3,5-bis-trifluoromethyl-phenyl)-1-methoxycarbonyl-ethylamino]-2-methanesulfinyl-pyrimidine-5-carboxylate (80 mg) is dissolved in tetrahydrofuran (2 ml), and thereto is added a 50% aqueous dimethylamine solution (1 ml), and the mixture is stirred at room temperature for 10 minutes. To the reaction mixture solution is added water, and the mixture is extracted with ethyl acetate, and the organic layer is washed with a saturated brine, dried over magnesium sulfate, and concentrated under reduced pressure. The resulting residue is purified by silica gel column chromatography (hexane:ethyl acetate=4:1→13:7). The resulting residue is dissolved in chloroform and thereto is added a 4N-hydrochloric acid in dioxane and the reaction mixture is concentrated under reduced pressure. To the resulting residue is added ether and the precipitated crystals are filtered to give 4-[2-(3,5-bis-trifluoromethyl-phenyl)-1-methoxycarbonyl-ethylamino]-2-dimethylamino-pyrimidine-5-carboxylic acid ethyl ester hydrochloride (64 mg). MS (m/z): 509 [M+H]⁺

### Example 42

The corresponding starting compound is treated in a similar manner to Example 41 to give the compound as listed in Table 13.

TABLE 13

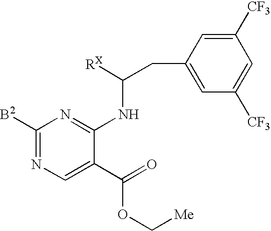

| | | | Physical |
|---|---|---|---|
| Ex. | | | properties, |
| No. | B²— | R^{X}— | etc. |
| 38 | 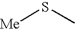 | 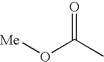 | MS (m/z):512 [M + H]⁺ |
| 39 | 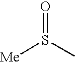 | 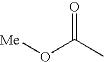 | MS (m/z):528 [M + H]⁺ |
| 40 | 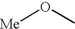 | 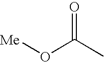 | MS (m/z):496 [M + H]⁺ |
| 41 | 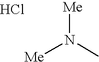 | 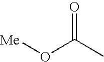 | MS (m/z):509 [M + H]⁺ |
| 42 | 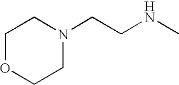 | 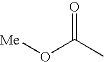 | MS (m/z):594 [M + H]⁺ |

### Examples 43 to 49

The corresponding starting compounds are treated in similar manners to any of the above Examples to give the compounds as listed in Table 14.

TABLE 14

| | | Physical |
|---|---|---|
| Ex. | | properties, |
| No. | Structural formula | etc. |
| 43 | 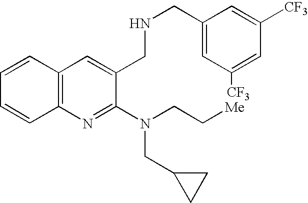 | MS (m/z):496[M + H]⁺ |
| 44 | 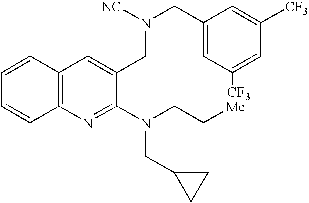 | MS (m/z):521[M + H]⁺ |
| 45 | 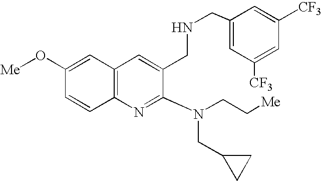 | MS (m/z):526[M + H]⁺ |
| 46 | 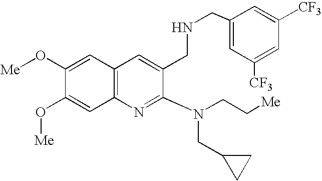 | MS (m/z):556[M + H]⁺ |
| 47 | 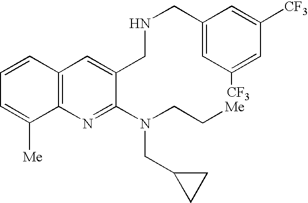 | MS (m/z):510[M + H]⁺ |
| 48 | 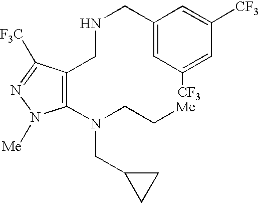 | MS (m/z):517[M + H]⁺ |
| 49 | 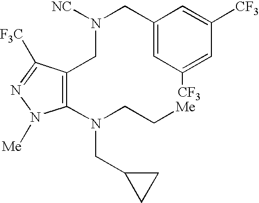 | MS (m/z):542[M + H]⁺ |

### Example 50

(1) A mixture of 2-chloroquinoline-3-carboxaldehyde (1.0 g), 2-benzyloxymethyl-5-tributylstannyl-2H-tetrazol (5.0 g) and dichlorobis(triphenylphosphine)palladium (II) (366 mg) in N,N-dimethylformamide (5 ml) is stirred at 100° C. under nitrogen atmosphere overnight. The reaction solution is cooled to room temperature, and thereto is added a saturated aqueous potassium fluoride solution, and the mixture is stirred at room temperature for 30 minutes. The insoluble materials are removed by filtration through Celite™, and the filtrate is extracted with ethyl acetate. The organic layer is washed successively with water and a saturated brine, dried over magnesium sulfate, and concentrated under reduced pressure. The resulting residue is purified by silica gel column chromatography (hexane:ethyl acetate=80:20→62:38) to give 3-(2-benzyloxymethyl-2H-tetrazol-5-yl)quinoline-3-carboxaldehyde (237 mg). MS (m/z): 346 [M+H]⁺

(2) 3-(2-Benzyloxymethyl-2H-tetrazol-5-yl)quinoline-3-carboxaldehyde (237 mg) is treated in similar manners to Examples 3(2)-(3) and 24 to give [2-(2-benzyloxymethyl-2H-tetrazol-5-yl)quinolin-3-ylmethyl]-(3,5-bis-trifluoromethylbenzyl)-(5-morpholin-4-yl-pyrimidin-2-yl)amine (59 mg). MS (m/z): 736 [M+H]⁺

TABLE 15

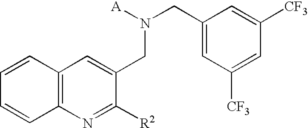

| | | | Physical |
|---|---|---|---|
| Ex. | | | properties, |
| No. | A— | —R² | etc. |
| 50 | 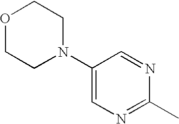 | 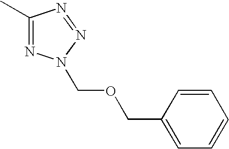 | MS (m/z):736[M + H]⁺ |

### Example 51

(1) 2-Hydroxymethyl-6-methylpyridin-3-ol (3.0 g) is dissolved in N,N-dimethylformamide (40 ml) and thereto are added benzyl bromide (3 ml) and potassium carbonate (3.58 g), and the mixture is stirred at room temperature overnight. To the reaction solution are added ethyl acetate and water, and the mixture is separated, and the organic layer is washed with a saturated brine, dried over magnesium sulfate, and concentrated under reduced pressure. The residue is purified by silica gel column chromatography (hexane:ethyl acetate=19:1→4:1) to give (3-benzyloxy-6-methyl-pyridin-2-yl)-methanol (4.45 g). MS (m/z): 230 [M+H]⁺

(2) (3-Benzyloxy-6-methyl-pyridin-2-yl)-methanol (4.45 g) is dissolved in methylene chloride (15 ml), and thereto are added carbon tetrabromide (7.08 g) and triphenylphosphine (5.34 g) under ice-cooling, and the mixture is stirred at 0° C. for 1 hour. The reaction solution is concentrated under reduced pressure and the resulting residue is purified by silica gel column chromatography (hexane:ethyl acetate=19:1→14:1) to give 3-benzyloxy-2-bromomethyl-6-methyl-pyridine (4.85 g). MS (m/z): 292/294 [M+H]⁺

(3) (3,5-Bis-trifluoromethyl-benzyl)-(5-bromo-pyrimidin-2-yl)-amine (6 mg) is dissolved in N,N-dimethylformamide (50 ml), and thereto is added sodium hydride (60%) (720 mg) under ice-cooling, and the mixture is stirred at 0° C. for 15 minutes. To the reaction solution is added 3-benzyloxy-2-bromomethyl-6-methyl-pyridine (4.85 g) and the mixture is stirred at room temperature for 1.5 hours. To the reaction solution are added water and ethyl acetate, and the mixture is separated, and the organic layer is washed with a saturated brine, dried over magnesium sulfate, and concentrated under reduced pressure. The resulting residue is purified by NH-silica gel column chromatography (hexane:ethyl acetate=19:1→17:3) to give (3-benzyloxy-6-methyl-pyridin-2-ylmethyl)-(3,5-bis-trifluoromethyl-benzyl)-(5-bromo-pyrimidin-2-yl)-amine (8.92 g). 611/613 [M+H]⁺

(4) (3-Benzyloxy-6-methyl-pyridin-2-ylmethyl)-(3,5-bis-trifluoromethyl-benzyl)-(5-bromo-pyrimidin-2-yl)-amine (1 g) is dissolved in toluene (15 ml), and thereto are added tris(dibenzylideneacetone)dipalladium (150 mg), sodium tert-butoxide (236 mg), 2-(di-tert-butylphosphino)biphenyl (196 mg), and ethyl piperidine-4-carboxylate (380 μl), and the mixture is stirred at room temperature under nitrogen flow for 3 days. To the reaction solution are added a saturated sodium bicarbonate and ethyl acetate, and the mixture is separated, and the organic layer is washed with a saturated brine, dried over magnesium sulfate, and concentrated under reduced pressure. The residue is purified by silica gel column chromatography (hexane:ethyl acetate=19:1→4:1→1:1) to give ethyl 1-{2-[(3-benzyloxy-6-methyl-pyridin-2-ylmethyl)-(3,5-bis-trifluoromethyl-benzyl)-amino]-pyrimidin-5-yl}-piperidine-4-carboxylate (234 mg). 688 [M+H]⁺

(5) Ethyl 1-{2-[(3-benzyloxy-6-methyl-pyridin-2-ylmethyl)-(3,5-bis-trifluoromethyl-benzyl)-amino]-pyrimidin-5-yl}-piperidine-4-carboxylate (230 mg) is dissolved in ethanol (5 ml), and thereto is added 10% palladium-carbon (50 mg) and the mixture is stirred at room temperature under hydrogen atmosphere for 5 hours. The catalyst is removed by filtration, and the filtrate is concentrated under reduced pressure. The resulting residue is purified by silica gel column chromatography (hexane:ethyl acetate=19:1→4:1) to give ethyl 1-{2-[(3,5-bis-trifluoromethyl-benzyl)-(3-hydroxy-6-methyl-pyridin-2-ylmethyl)-amino]-pyrimidin-5-yl}-piperidine-4-carboxylate (152 mg). MS (m/z): 598 [M+H]⁺

(6) Ethyl 1-{2-[(3,5-bis-trifluoromethyl-benzyl)-(3-hydroxy-6-methyl-pyridin-2-ylmethyl)-amino]-pyrimidin-5-yl}-piperidine-4-carboxylate (152 mg) and pyridine (31 μl) are dissolved in methylene chloride (4 ml), and thereto is added trifluoromethanesulfonic anhydride (65 μl) under ice-cooling, and the mixture is stirred at 0° C. under nitrogen atmosphere for 1 hour, and to the reaction solution is added trifluoromethanesulfonic anhydride (65 μl) and the mixture is cooled to the room temperature and stirred for 1 hour. To the reaction solution are added a saturated aqueous sodium bicarbonate solution and chloroform, and the mixture is separated, and the organic layer is washed with a saturated brine, dried over magnesium sulfate, and concentrated under reduced pressure. The residue is purified by silica gel column chromatography (hexane:ethyl acetate=19:1→4:1→3:2) to give ethyl 1-{2-[(3,5-bis-trifluoromethyl-benzyl)-(6-methyl-3-trifluoromethanesulfonyloxy-pyridin-2-ylmethyl)-amino]-pyrimidin-5-yl}-piperidine-4-carboxylate (147 mg). MS (m/z): 730 [M+H]⁺

(7) Ethyl 1-{2-[(3,5-bis-trifluoromethyl-benzyl)-(6-methyl-3-trifluoromethanesulfonyloxy-pyridin-2-ylmethyl)-amino]-pyrimidin-5-yl}-piperidine-4-carboxylate (143 mg) is dissolved in 1,4-dioxane (4 ml), and thereto are added 5-isopropyl-2-methoxy-phenyl boronic acid (57 mg), [1,1′-bis(diphenylphosphino)ferrocene]dichloropalladium dichloromethane complex (16 mg) and cesium carbonate (96 mg), and the mixture is stirred at 80° C. under nitrogen atmosphere for 2.5 hours. The reaction solution is cooled to room temperature, and thereto are added water and ethyl acetate, and the mixture is separated. The organic layer is washed with a saturated brine, dried over magnesium sulfate, and concentrated under reduced pressure. The resulting residue is purified by silica gel column chromatography (hexane:ethyl acetate=19:14→:1) to give ethyl 1-(2-{(3,5-bis-trifluoromethyl-benzyl)-[3-(5-isopropyl-2-methoxy-phenyl)-6-methyl-pyridin-2-ylmethyl]-amino}-pyrimidin-5-yl)-piperidine-4-carboxylate (97 mg). MS (m/z): 730 [M+H]⁺

(8) Ethyl 1-(2-{(3,5-bis-trifluoromethyl-benzyl)-[3-(5-isopropyl-2-methoxy-phenyl)-6-methyl-pyridin-2-ylmethyl]-amino}-pyrimidin-5-yl)-piperidine-4-carboxylate (92 mg) is dissolved in ethanol (4 ml), and thereto is added a 1N-aqueous sodium hydroxide solution (1 ml) and the mixture is stirred at room temperature for 1 day. To the reaction solution are added a 1N-hydrochloric acid and ethyl acetate, and the mixture is separated, and the organic layer is washed with a saturated brine, dried over magnesium sulfate, and concentrated under reduced pressure. The resulting residue is purified by silica gel column chromatography (chloroform:methanol=1:0→9:1) to give 1-(2-{(3,5-bis-trifluoromethyl-benzyl)-[3-(5-isopropyl-2-methoxy-phenyl)-6-methyl-pyridin-2-ylmethyl]-amino}-pyrimidin-5-yl)-piperidine-4-carboxylic acid (73 mg). MS (m/z): 702 [M+H]⁺

### Example 52

(1) (3-Benzyloxy-6-methylpyridin-2-ylmethyl)-(3,5-bis-trifluoromethyl-benzyl)-(5-bromo-pyrimidin-2-yl)-amine (5 g) is treated in similar manners to Examples 5(1)-(2) to give ethyl 4-{2-[(3-benzyloxy-6-methyl-pyridin-2-ylmethyl)-(3,5-bis-trifluoromethyl-benzyl)-amino]-pyrimidin-5-yloxy}-butyrate (1.69 g). MS (m/z): 663 [M+H]⁺

(2) Ethyl 4-{2-[(3-benzyloxy-6-methyl-pyridin-2-ylmethyl)-(3,5-bis-trifluoromethyl-benzyl)-amino]-pyrimidin-5-yloxy}-butyrate (1.67 g) is treated in similar manners to Examples 51(5)-(6) to give ethyl 4-{2-[(3,5-bis-trifluoromethyl-benzyl)-(6-methyl-3-trifluoromethanesulfonyloxy-pyridin-2-ylmethyl)-amino]-pyrimidin-5-yloxy}-butyrate (1.4 g). MS (m/z): 705 [M+H]⁺

(3) Ethyl 4-{2-[(3,5-bis-trifluoromethyl-benzyl)-(6-methyl-3-trifluoromethanesulfonyloxy-pyridin-2-ylmethyl)-amino]-pyrimidin-5-yloxy}-butyrate (313 mg) is treated in similar manners to Examples 51(7)-(8) to give 4-(2-{(3,5-bis-trifluoromethyl-benzyl)-[3-(5-isopropyl-2-methoxy-phenyl)-6-methyl-pyridin-2-ylmethyl]-amino}-pyrimidin-5-yloxy)-butyrate (74 mg). MS (m/z): 677 [M+H]⁺

TABLE 16

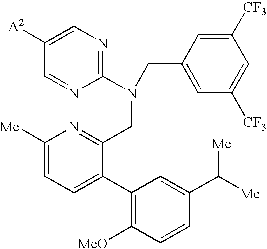

| | | Physical |
|---|---|---|
| Ex. | | properties, |
| No. | A²— | etc. |
| 51 | 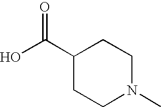 | MS (m/z):702[M + H]⁺ |
| 52 | 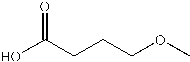 | MS (m/z):677[M + H]⁺ |

### Example 53

(1) (3,5-Bis-trifluoromethyl-benzyl)-(5-bromopyrimidin-2-yl)-[2-(5-isopropyl-2-methoxy-phenyl)-8-methyl-quinolin-3-ylmethyl]amine (200 mg) is dissolved in dioxane (1.5 ml), and thereto are added tris(dibenzylideneacetone)dipalladium (39 mg), tetrafluoroboric acid tri-tert-butylphosphonium (25 mg), methyl acrylate (51 μl) and N,N-diisopropylethylamine (74 μl), and the mixture is degassed under reduced pressure and stirred at 50° C. under nitrogen flow for 5 hours. The reaction solution is allowed to cool to room temperature, and thereto is added a saturated brine, and the mixture is extracted with ethyl acetate and the organic layer is washed with a saturated brine, dried over magnesium sulfate, and concentrated under reduced pressure. The resulting residue is purified by silica gel column chromatography (hexane:ethyl acetate=91:9→4:1) to give methyl 3-(2-{(3,5-bis-trifluoromethyl-benzyl)-[2-(5-isopropyl-2-methoxy-phenyl)-8-methyl-quinolin-3-ylmethyl]amino}-pyrimidin-5-yl)-acrylate (152 mg). MS (m/z): 709 [M+H]⁺

(2) Methyl 3-(2-{(3,5-bis-trifluoromethyl-benzyl)-[2-(5-isopropyl-2-methoxy-phenyl)-8-methyl-quinolin-3-ylmethyl]amino}-pyrimidin-5-yl)-acrylate (149 mg) is dissolved in a mixed solvent of methanol (0.5 ml) and tetrahydrofuran (2 ml), and thereto is added a 1M-aqueous sodium hydroxide solution (0.5 ml) and the mixture is stirred at room temperature for 4 hours. The reaction mixture is made acidic with a 10% aqueous citric acid solution and the mixture is extracted with ethyl acetate. The organic layer is washed with a saturated brine, dried over magnesium sulfate, and concentrated under reduced pressure to give 3-(2-{(3,5-bis-trifluoromethyl-benzyl)-[2-(5-isopropyl-2-methoxy-phenyl)-8-methyl-quinolin-3-ylmethyl]amino}-pyrimidin-5-yl)-acrylic acid (151 mg). MS (m/z): 695 [M+H]⁺

### Examples 54 to 56

The corresponding starting compounds are treated in a similar manner to Example 4 to give the desired compounds.

### Example 57

(1) (3-{[(3,5-Bis-trifluoromethyl-benzyl)-(5-bromo-pyrimidin-2-yl)-amino]-methyl}-8-methyl-quinolin-2-yl)-cyclopropylmethyl-propyl-amine (300 mg) is dissolved in toluene (5 ml), and thereto are added tris(dibenzylideneacetone)dipalladium (41 mg), sodium tert-butoxide (65 mg), 2-(di-tert-butylphosphino)biphenyl (54 mg) and tert-butyl 3-methylamino-propionate (107 mg) and the mixture is stirred at room temperature under nitrogen flow overnight. To the reaction solution are added water and diethyl ether, and the mixture is separated, and the organic layer is dried over magnesium sulfate, and concentrated under reduced pressure. The resulting residue is purified by NH-silica gel column chromatography (hexane:ethyl acetate=1:0→85:15) to give tert-butyl 3-[(2-{(3,5-bis-trifluoromethyl-benzyl)-[2-(cyclopropylmethyl-propyl-amino)-8-methyl-quinolin-3-ylmethyl]-amino}-pyrimidin-5-yl)-methyl-amino]-propionate (121 mg). MS (m/z): 745 [M+H]⁺

(2) Tert-butyl 3-[(2-{(3,5-bis-trifluoromethyl-benzyl)-[2-(cyclopropylmethyl-propyl-amino)-8-methyl-quinolin-3-ylmethyl]-amino}-pyrimidin-5-yl)-methyl-amino]-propionate (115 mg) is dissolved in a 4N-hydrochloric acid in ethyl acetate (3 ml) and the mixture is stirred at room temperature for 3 hours. To the reaction solution are added water and a saturated aqueous sodium bicarbonate solution to make the pH of aqueous layer to be about 4, and the mixture is extracted with diethyl ether. The organic layer is washed with a saturated brine, dried over magnesium sulfate, and concentrated under reduced pressure. The resulting residue is purified by silica gel column chromatography (chloroform:methanol=49:1→93:7) to give 3-[(2-{(3,5-bis-trifluoromethyl-benzyl)-[2-(cyclopropylmethyl-propyl-amino)-8-methyl-quinolin-3-ylmethyl]-amino}-pyrimidin-5-yl)-methyl-amino]-propionic acid (86 mg). MS (m/z): 689 [M+H]⁺

### Example 58

(1) 2-{(3,5-Bis-trifluoromethyl-benzyl)-[2-(5-isopropyl-2-methoxy-phenyl)-8-methyl-quinolin-3-ylmethyl]amino}-pyrimidin-5-ol (88 mg) is dissolved in tetrahydrofuran (1 ml), and thereto are added 2-(methylthio)ethanol (25 mg), and triphenylphosphine (72 mg), followed by an addition of a 40% diethyl azodicarboxylate/toluene (119 μl) under water-cooling, and the mixture is stirred at room temperature for 2 hours. To the reaction mixture is added a saturated brine, and the mixture is extracted with ethyl acetate. The organic layer is washed with a saturated brine, dried over magnesium sulfate, and concentrated under reduced pressure. The resulting residue is purified by silica gel column chromatography (hexane:ethyl acetate=1:0→4:1) to give (3,5-bis-trifluoromethyl-benzyl)-[2-(5-isopropyl-2-methoxy-phenyl)-8-methyl-quinolin-3-ylmethyl]-[5-(2-methylsulfanyl-ethoxy)-pyrimidin-2-yl]-amine (117 mg). MS (m/z): 715 [M+H]⁺

(2) (3,5-Bis-trifluoromethyl-benzyl)-[2-(5-isopropyl-2-methoxy-phenyl)-8-methyl-quinolin-3-ylmethyl]-[5-(2-methylsulfanyl-ethoxy)-pyrimidin-2-yl]-amine (114 mg) is dissolved in chloroform (2 ml), and thereto is added m-chloroperbenzoic acid (44 mg) and the mixture is stirred at room temperature for 2 hours. To the reaction solution are added a saturated aqueous sodium bicarbonate solution and the mixture is extracted with ethyl acetate and the organic layer is washed successively with a saturated aqueous sodium bicarbonate solution and a saturated brine, dried over magnesium sulfate, and concentrated under reduced pressure. The resulting residue is purified by silica gel column chromatography (hexane:ethyl acetate=1:1, and chloroform:methanol=19:1) to give (3,5-bis-trifluoromethyl-benzyl)-[2-(5-isopropyl-2-methoxy-phenyl)-8-methyl-quinolin-3-ylmethyl]-[5-(2-methanesulfonyl-ethoxy)-pyrimidin-2-yl]-amine (63 mg). MS (m/z): 747 [M+H]⁺

TABLE 17

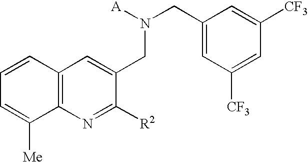

| | | | Physical |
|---|---|---|---|
| Ex. | | | properties, |
| No. | A— | —R² | etc. |
| 53 | 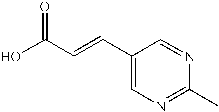 | 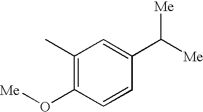 | MS (m/z):695[M + H]⁺ |
| 54 | 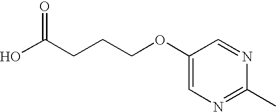 | 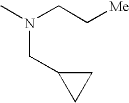 | MS (m/z):690[M + H]⁺ |
| 55 | 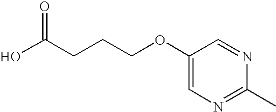 | 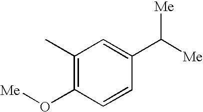 | MS (m/z):727[M + H]⁺ |
| 56 | 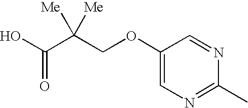 | 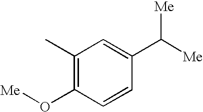 | MS (m/z):741[M + H]⁺ |
| 57 | 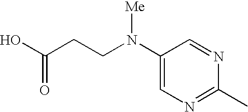 | 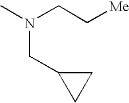 | MS (m/z):689[M + H]⁺ |
| 58 | 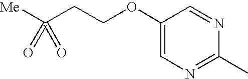 | 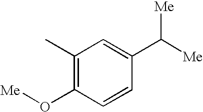 | MS (m/z):747[M + H]⁺ |

### Example 59

(1) Benzyl alcohol (22.7 ml) is dissolved in tetrahydrofuran (180 ml), and thereto is added stepwise sodium hydride (60%) (8.76 g) under ice-cooling, and the mixture is stirred at room temperature for 20 minutes. To this reaction mixture is added a solution of 2-chloro-8-methyl-quinoline-3-carbaldehyde (15.00 g) dissolved in tetrahydrofuran (180 ml), and the mixture is stirred at 50° C. for 5 hours. To the reaction mixture is added a 10% aqueous citric acid solution and a saturated brine and the mixture is extracted with ethyl acetate. The organic layer is washed with a saturated brine, dried over magnesium sulfate, and concentrated under reduced pressure. To the resulting residue is added ether, and the insoluble materials are removed by filteration and the filtrate is concentrated under reduced pressure. The resulting residue is purified by silica gel column chromatography (hexane:ethyl acetate=19:1→3:1). To the resulting residue is added isopropylether and the precipitated crystals are filtered to give (2-benzyloxy-8-methyl-quinolin-3-yl)-methanol (2.62 g). MS (m/z): 280 [M+H]⁺

(2) (2-Benzyloxy-8-methyl-quinolin-3-yl)-methanol (3.59 g) is treated in similar manners to Example 51(2)-(3) to give (2-benzyloxy-8-methyl-quinolin-3-ylmethyl)-(3,5-bis-trifluoromethyl-benzyl)-(5-bromo-pyrimidin-2-yl)-amine (5.39 g). MS (m/z): 663/661 [M+H]⁺

(3) (2-Benzyloxy-8-methyl-quinolin-3-ylmethyl)-(3,5-bis-trifluoromethyl-benzyl)-(5-bromo-pyrimidin-2-yl)-amine (4.73 g) is treated with the corresponding starting compound in a similar manner to Example 52 to give 4-(2-{(3,5-bis-trifluoromethyl-benzyl)-[2-(2,5-dimethoxy-phenyl)-8-methyl-quinolin-3-ylmethyl]-amino}-pyrimidin-5-yloxy)-butyric acid (66 mg). MS (m/z): 713 [M-Na]⁻

### Example 60

The corresponding starting compound is treated in a similar manner to Example 59 to give the desired compound.

### Example 61

(1) 2-[(2-Benzyloxy-8-methyl-quinolin-3-ylmethyl)-(3,5-bis-trifluoromethyl-benzyl)-amino]-pyrimidin-5-ol (700 mg) is treated in a similar manner to Example 58 to give (2-benzyloxy-8-methyl-quinolin-3-ylmethyl)-(3,5-bis-trifluoromethyl-benzyl)-[5-(2-methanesulfonyl-ethoxy)-pyrimidin-2-yl]-amine (607 mg). MS (m/z): 705 [M+H]⁺

(2) (2-Benzyloxy-8-methyl-quinolin-3-ylmethyl)-(3,5-bis-trifluoromethyl-benzyl)-[5-(2-methanesulfonyl-ethoxy)-pyrimidin-2-yl]-amine (546 mg) is dissolved in methylene chloride (5 ml), and thereto is added a 4N-hydrochloric acid in dioxane (5 ml), and the mixture is stirred at room temperature for 1 hour. The reaction solution is concentrated under reduced pressure, and the residue is neutralized with a saturated aqueous sodium bicarbonate solution, and the mixture is made weakly acidic with a 10% aqueous citric acid solution and extracted with a mixed solution of ethyl acetate and a small amount of methylene chloride. The organic layer is washed with a saturated brine, dried over magnesium sulfate, and concentrated under reduced pressure to give 3-({(3,5-bis-trifluoromethyl-benzyl)-[5-(2-methanesulfonyl-ethoxy)-pyrimidin-2-yl]-amino}-methyl)-8-methyl-quinolin-2-ol (530 mg). MS (m/z): 615 [M+H]⁺

(3) 3-({(3,5-Bis-trifluoromethyl-benzyl)-[5-(2-methanesulfonyl-ethoxy)-pyrimidin-2-yl]-amino}-methyl)-8-methyl-quinolin-2-ol (462 mg) is dissolved in methylene chloride (8 ml), and thereto is added triethylamine (630 μl), followed by an addition stepwise of trifluoromethanesulfonic anhydride (380 μl) under water-cooling, and the mixture is stirred at room temperature for 2 hours. To the reaction mixture is added a saturated brine, and the mixture is extracted with ethyl acetate and the organic layer is washed with a saturated brine, dried over magnesium sulfate, and concentrated under reduced pressure. The resulting residue is purified by silica gel column chromatography (hexane:ethyl acetate=19:1→13:7) to give trifluoromethanesulfonic acid 3-({(3,5-bis-trifluoromethyl-benzyl)-[5-(2-methanesulfonyl-ethoxy)-pyrimidin-2-yl]-amino}-methyl)-8-methyl-quinolin-2-ylester (427 mg). MS (m/z): 747 [M+H]⁺

(4) Trifluoromethanesulfonic acid 3-({(3,5-bis-trifluoromethyl-benzyl)-[5-(2-methanesulfonyl-ethoxy)-pyrimidin-2-yl]-amino}-methyl)-8-methyl-quinolin-2-ylester (100 mg) is treated with the corresponding starting compound in a similar manner to Example 51(7) to give (3,5-bis-trifluoromethyl-benzyl)-[2-(2,4-dimethoxy-pyrimidin-5-yl)-8-methyl-quinolin-3-ylmethyl]-[5-(2-methanesulfonyl-ethoxy)-pyrimidin-2-yl]-amine (45 mg). MS (m/z): 737 [M+H]⁺

### Example 62

(1) (3,5-Bis-trifluoromethyl-benzyl)-[2-(5-isopropyl-2-methoxy-phenyl)-8-methyl-quinolin-3-ylmethyl]-(1H-tetrazol-5-yl)-amine (which is obtained by treating the corresponding starting compound in a similar manner to Example 6) (100 mg) is dissolved in N,N-dimethylformamide (10 ml), and thereto are added potassium carbonate (67 mg) and ethyl 4-bromo-butyrate (70 μl) and the mixture is stirred at 60° C. for 3 hours. The reaction solution is allowed to cool to room temperature, and thereto are added a 10% aqueous citric acid solution and a saturated brine, and the mixture is extracted with ethyl acetate. The organic layer is washed with a saturated brine, dried over magnesium sulfate, and concentrated under reduced pressure. The resulting residue is purified by silica gel column chromatography (hexane:ethyl acetate=99:1→17:3) to give ethyl 4-(5-{(3,5-bis-trifluoromethyl-benzyl)-[2-(5-isopropyl-2-methoxyphenyl)-8-methyl-quinolin-3-ylmethyl]-amino}-tetrazol-2-yl)-butyrate (111 mg). MS (m/z): 729 [M+H]⁺

(2) Ethyl 4-(5-{(3,5-bis-trifluoromethyl-benzyl)-[2-(5-isopropyl-2-methoxy-phenyl)-8-methyl-quinolin-3-ylmethyl]-amino}-tetrazol-2-yl)-butyrate (96 mg) is treated in a similar manner to Example 1(3) to give 4-(5-{(3,5-bis-trifluoromethyl-benzyl)-[2-(5-isopropyl-2-methoxy-phenyl)-8-methyl-quinolin-3-ylmethyl]-amino}-tetrazol-2-yl)-butyric acid (99 mg). MS (m/z): 701 [M+H]⁺

TABLE 18

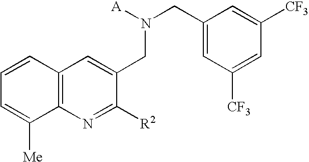

| | | | Physical |
|---|---|---|---|
| Ex. | | | properties, |
| No. | A— | —R² | etc. |
| 59 | 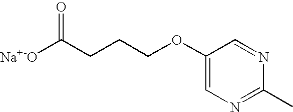 | 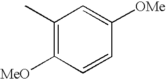 | MS (m/z):713[M − Na]⁻ |
| 60 | 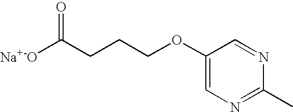 | 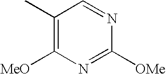 | MS (m/z):715[M − Na]⁻ |
| 61 | 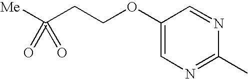 | 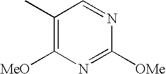 | MS (m/z):735[M + H]⁺ |
| 62 | 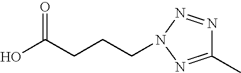 | 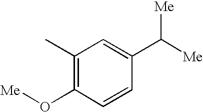 | MS (m/z):701[M + H]⁺ |

### Example 63

(1) To tetrahydrofuran (30 ml) is added a 1.6M n-butyl lithium/hexane (10.9 ml), followed by an addition dropwise of diisopropylamine (2.45 ml) under nitrogen atmosphere at −78° C., and the mixture is stirred at 0° C. for 1 hour. To the reaction solution is added dropwise a solution of 2,8-dichloro-quinoline (3.15 g) dissolved in tetrahydrofuran (10 ml) at −78° C., and the mixture is stirred at the same temperature for 2 hours. To the reaction solution are added dropwise ethyl formate (5.1 ml) at −78° C., and the mixture is stirred for 1 hour, and thereto are added an aqueous ammonium chloride solution and the mixture is extracted with ethyl acetate. The organic layer is washed with a saturated brine, dried over magnesium sulfate, and concentrated under reduced pressure, and to the resulting residue are added ethyl acetate and ether and the precipitated crystals are filtered to give 2,8-dichloro-quinoline-3-carbaldehyde (1.21 g). MS (m/z): 228/226 [M+H]⁺

(2) 2,8-Dichloro-quinoline-3-carbaldehyde (1.18 g) is treated in similar manners to Examples 9 and 4 to give 1-(2-{(3,5-bis-trifluoromethyl-benzyl)-[8-chloro-2-(cyclopropylmethyl-propyl-amino)-quinolin-3-ylmethyl]-amino}-pyrimidin-5-yl)-piperazine-4-carboxylic acid (87 mg). MS (m/z): 737/735 [M+H]⁺

TABLE 19

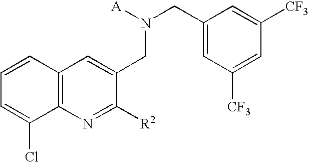

| | | | Physical |
|---|---|---|---|
| Ex. | | | properties, |
| No. | A— | —R² | etc. |
| 63 | 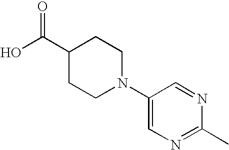 | 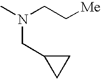 | MS (m/z):735/737[M + H]⁺ |

### Example 64

(3,5-Bis-trifluoromethyl-benzyl)-(5-bromo-pyrimidin-2-yl)-quinolin-8-ylmethyl-amine (300 mg) (which is obtained by treating the corresponding starting compound in similar manners to Examples 25(4)-(5)) is treated in a similar manner to Example 4 to give 1-{2-[(3,5-bis-trifluoromethyl-benzyl)-quinolin-8-ylmethyl-amino]-pyrimidin-5-yl}-piperidine-4-carboxylic acid (16 mg). MS (m/z): 590 [M+H]⁺

TABLE 20

| | | Physical |
|---|---|---|
| Ex. | | properties, |
| No. | Structural formula | etc. |
| 64 | 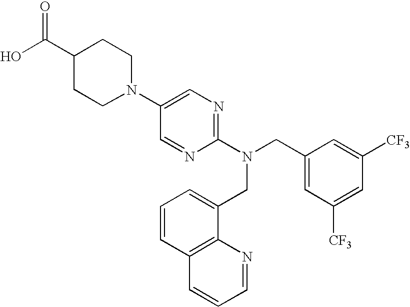 | MS (m/z):590[M + H]⁺ |

### Example 65

(1) [2-(5-Isopropyl-2-methoxy-phenyl)-8-methyl-quinolin-3-yl]-methanol (which is obtained by treating the corresponding starting compound in similar manners to Examples 3(1)-(2)) (2.06 g) is dissolved in methylene chloride (10 ml), and thereto is added dropwise thionyl chloride (545 μl) at 0° C. The mixture is stirred at room temperature for 15 minutes and concentrated under reduced pressure, and the resulting residue is dissolved in methylene chloride and concentrated under reduced pressure again to give 3-chloromethyl-2-(5-isopropyl-2-methoxy-phenyl)-8-methyl-quinoline (2.39 g). MS (m/z): 342/340 [M+H]⁺

(2) To 3-chloromethyl-2-(5-isopropyl-2-methoxy-phenyl)-8-methyl-quinoline (2.39 g) are added N,N-dimethylformamide (12 ml), phthalimide potassium salt (2.54 g) and potassium carbonate (2.58 g), and the mixture is stirred at 80° C. for 4 hours. The reaction solution is allowed to cool to room temperature, and thereto are added a saturated brine and a 10% aqueous citric acid solution, and the mixture is extracted with ethyl acetate. The organic layer is washed twice with a saturated brine, dried over magnesium sulfate, and concentrated under reduced pressure. To the resulting residue is added ether and the mixture is filtered and the filtrate is concentrated under reduced pressure. The resulting residue is purified by silica gel column chromatography (hexane:ethyl acetate=19:1→3:1) to give 2-[2-(5-isopropyl-2-methoxy-phenyl)-8-methyl-quinolin-3-ylmethyl]-isoindole-1,3-dione (2.35 g). MS (m/z): 451 [M+H]⁺

(3) 2-[2-(5-Isopropyl-2-methoxy-phenyl)-8-methyl-quinolin-3-ylmethyl]-isoindole-1,3-dione (2.34 g) is dissolved in ethanol (25 ml) and thereto is added hydrazine anhydrous (200 mg), and the mixture is stirred at 80° C. for 3 hours and a half. The reaction solution is allowed to cool to room temperature, and thereto is added brine and the mixture is extracted with ethyl acetate. The organic layer is washed with a saturated brine, dried over magnesium sulfate, and concentrated under reduced pressure. The resulting residue is purified by silica gel column chromatography (chloroform:methanol=1:0→19:1) to give C-[2-(5-isopropyl-2-methoxy-phenyl)-8-methyl-quinolin-3-yl]-methylamine (1.54 g). MS (m/z): 321 [M+H]⁺

(4) C-[2-(5-isopropyl-2-methoxy-phenyl)-8-methyl-quinolin-3-yl]-methylamine (760 mg) is dissolved in toluene (12 ml), and thereto are added tert-butyl 4-(2-chloro-pyrimidin-5-yloxy)-butyrate (838 mg), palladium acetate(II) (53 mg), (±)-2,2′-bis(diphenylphosphino)-1,1′-binaphthalene (148 mg), tetra-n-butylammonium iodide (175 mg) and sodium tert-butoxide (274 mg), and the mixture is stirred at 85° C. under nitrogen flow overnight. The reaction solution is allowed to cool to room temperature, and thereto is added water and the mixture is extracted with chloroform, and the organic layer is dried over magnesium sulfate and concentrated under reduced pressure. The resulting residue is purified by silica gel column chromatography (hexane:ethyl acetate=4:1) to give tert-butyl 4-(2-{[2-(5-isopropyl-2-methoxy-phenyl)-8-methyl-quinolin-3-ylmethyl]-amino}-pyrimidin-5-yloxy)-butyrate (415 mg). MS (m/z): 557 [M+H]⁺

(5) Tert-butyl 4-(2-{[2-(5-isopropyl-2-methoxy-phenyl)-8-methyl-quinolin-3-ylmethyl]-amino}-pyrimidin-5-yloxy)-butyrate (187 mg) is dissolved in N,N-dimethylformamide (1.5 ml), and thereto are added sodium hydride (60%) (20 mg) and 3-bromomethyl-5-trifluoromethyl-benzonitrile (266 mg) under water-cooling, and the mixture is stirred at room temperature for 30 minutes, and thereto are added sodium hydride (60%) (60 mg) and 3-bromomethyl-5-trifluoromethyl-benzonitrile (100 mg) again, and the mixture is stirred at room temperature for 2 hours and a half. To the reaction solution is added water and the mixture is extracted with chloroform and the organic layer is dried over magnesium sulfate and concentrated under reduced pressure. The resulting residue is purified by silica gel column chromatography (hexane:ethyl acetate=4:1) to give tert-butyl 4-(2-{(3-cyano-5-trifluoromethyl-benzyl)-[2-(5-isopropyl-2-methoxy-phenyl)-8-methyl-quinolin-3-ylmethyl]-amino}-pyrimidin-5-yloxy)-butyrate (144 mg). MS (m/z): 740 [M+H]⁺

(6) To tert-butyl 4-(2-{(3-cyano-5-trifluoromethyl-benzyl)-[2-(5-isopropyl-2-methoxy-phenyl)-8-methyl-quinolin-3-ylmethyl]-amino}-pyrimidin-5-yloxy)-butyrate (135 mg) is added a 4N-hydrochloric acid in dioxane (2 ml), and the mixture is stirred at room temperature for 2 hours. To the reaction mixture is added a saturated brine, and the mixture is extracted with ethyl acetate and the organic layer is dried over magnesium sulfate and concentrated under reduced pressure. The resulting residue is purified by silica gel column chromatography (chloroform:methanol=1:0→9:1) to give 4-(2-{(3-cyano-5-trifluoromethyl-benzyl)-[2-(5-isopropyl-2-methoxy-phenyl)-8-methyl-quinolin-3-ylmethyl]-amino}-pyrimidin-5-yloxy)-butyrate (115 mg). MS (m/z): 684 [M+H]⁺

TABLE 21

| | | Physical |
|---|---|---|
| Ex. | | properties, |
| No. | Structural formula | etc. |
| 65 | 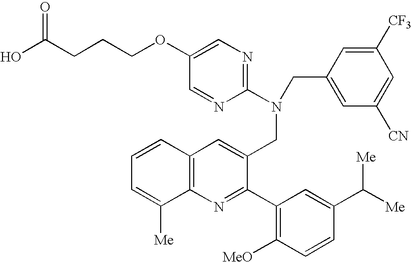 | MS (m/z):684[M + H]⁺ |

### Example 66

(1) Phthalaldehydic acid (2.5 g) is dissolved in N,N-dimethylformamide (300 ml) and thereto are added potassium carbonate (50.8 g) and di-tert-butyl bromomalonate (54 g), and the mixture is stirred at room temperature overnight. To the reaction solution are added water and diethyl ether, and the mixture is separated, and the organic layer is dried over magnesium sulfate and concentrated under reduced pressure. The resulting residue is dissolved in a 4N-hydrochloric acid/ethyl acetate (200 ml) and the mixture is stirred at room temperature overnight. The solvent is evaporated under reduced pressure and the residue is dissolved in acetic acid (100 ml) and 1,4-dioxane (500 ml), and the mixture is heated under reflux overnight. The reaction solution is concentrated under reduced pressure and the precipitated crystals are filtered with diisopropylether to give 1-oxo-1H-isochromene-3-carboxylic acid (17.5 g). MS (m/z): 191 [M+H]⁺

(2) 1-Oxo-1H-isochromene-3-carboxylic acid (17.5 g) is dissolved in 7N-ammonia/methanol solution (350 ml) and the mixture is stirred at room temperature overnight. The solvent is evaporated under reduced pressure and the residue is dissolved in a 4N-hydrochloric acid/dioxane (300 ml) and the mixture is stirred at room temperature overnight. The reaction solution is concentrated under reduced pressure and The precipitated crystals are filtered with diisopropylether to give 1-oxo-1,2-dihydroisoquinoline-3-carboxylic acid (19.0 g). MS (m/z): 190 [M+H]⁺

(3) The mixture of 1-oxo-1,2-dihydroisoquinoline-3-carboxylic acid (19.0 g), a concentrated sulfuric acid (100 ml) and methanol (500 ml) is heated under reflux overnight. The reaction solution is poured into ice-water and the mixture is extracted with chloroform. The organic layer is washed with a saturated brine, dried over magnesium sulfate, and concentrated under reduced pressure. The precipitated crystals are filtered with diisopropylether to give methyl 1-oxo-1,2-dihydroisoquinoline-3-carboxylate (15.4 g). MS (m/z): 204 [M+H]⁺

(4) The mixture of methyl 1-oxo-1,2-dihydroisoquinoline-3-carboxylate (15.3 g), benzyl bromide (10.75 ml), silver carbonate (31 g) and toluene (300 ml) is stirred at 100° C. for 2 hours. The insoluble materials are removed by filtration through Celite™, and the filtrate is concentrated under reduced pressure. Crystallization of the residue from diisopropylether gives methyl 1-benzyloxy-isoquinoline-3-carboxylate (21.3 g). MS (m/z): 294 [M+H]⁺

(5) The mixture of methyl 1-benzyloxy-isoquinoline-3-carboxylate (20.6 g), sodium borohydride (15.9 g) and tetrahydrofuran (500 ml) is stirred at 70° C. for 15 minutes. Then to the reaction solution are added dropwise methanol (100 ml) over 1 hour, and the mixture is stirred for additional 1 hour. The reaction solution is cooled to room temperature, and thereto is added a saturated aqueous ammonium chloride solution to consume the excess reagents and the mixture is extracted with chloroform. The organic layer is washed with a saturated brine, dried over magnesium sulfate, and concentrated under reduced pressure. Crystallization of the residue from diisopropylether-hexane gives (1-benzyloxy-isoquinolin-3-yl)-methanol (17.3 g). MS (m/z): 266 [M+H]⁺

(6) (1-Benzyloxy-isoquinolin-3-yl)-methanol (500 mg) is dissolved in methylene chloride (10 ml), and thereto is added thionyl chloride (151 μl) under ice-cooling and the mixture is stirred at the same temperature for 15 minutes and concentrated under reduced pressure. The resulting residue and (3,5-bis-trifluoromethyl-benzyl)-(5-bromo-pyrimidin-2-yl)-amine (752 mg) is dissolved in N,N-dimethylformamide (5 ml), and thereto is added sodium hydride (60%) (165 mg) under ice-cooling, and the mixture is stirred at room temperature overnight. To the reaction solution are added diethyl ether and water, and the mixture is separated, and the organic layer is washed with a saturated brine, dried over magnesium sulfate, and concentrated under reduced pressure. The resulting residue is purified by silica gel column chromatography (hexane:ethyl acetate=1:0→19:5) and the resulting crude product is recrystallized from diisopropylether to give (1-benzyloxy-isoquinolin-3-ylmethyl)-(3,5-bis-trifluoromethyl-benzyl)-(5-bromo-pyrimidin-2-yl)-amine (317 mg). MS (m/z): 647/649 [M+H]⁺

(7) (1-Benzyloxy-isoquinolin-3-ylmethyl)-(3,5-bis-trifluoromethyl-benzyl)-(5-bromo-pyrimidin-2-yl)-amine (6.35 g) is dissolved in toluene (100 ml), and thereto are added tris(dibenzylideneacetone)dipalladium (898 mg), sodium tert-butoxide (1.41 g), 2-(di-tert-butylphosphino)biphenyl (1.17 g) and ethyl isonipecotate (2.2 ml), and the mixture is stirred at room temperature under nitrogen atmosphere overnight. To the reaction solution are added water and ethyl acetate, and the mixture is separated, and the organic layer is dried over magnesium sulfate and concentrated under reduced pressure. The resulting residue is purified by NH-silica gel column chromatography (hexane:ethyl acetate=1:0→9:1) to give ethyl 1-{2-[(1-benzyloxy-isoquinolin-3-ylmethyl)-(3,5-bis-trifluoromethyl-benzyl)-amino]-pyrimidin-5-yl}-piperidine-4-carboxylate (3.04 g). MS (m/z): 724 [M+H]⁺

(8) Ethyl 1-{2-[(1-benzyloxy-isoquinolin-3-ylmethyl)-(3,5-bis-trifluoromethyl-benzyl)-amino]-pyrimidin-5-yl}-piperidine-4-carboxylate (2.88 g) is dissolved in a mixed solvent of ethanol (10 ml) and tetrahydrofuran (30 ml), and thereto is added 10% palladium-carbon (500 mg) and the mixture is stirred under hydrogen atmosphere at room temperature overnight. The catalyst is removed by filtration, and the filtrate is concentrated under reduced pressure and the resulting residue is purified by silica gel column chromatography (hexane:ethyl acetate=4:1→1:4) to give ethyl 1-{2-[(3,5-bis-trifluoromethyl-benzyl)-(1-oxo-1,2-dihydroisoquinolin-3-ylmethyl)-amino]-pyrimidin-5-yl}-piperidine-4-carboxylate (1.80 g). MS (m/z): 634 [M+H]⁺

(9) Ethyl 1-{2-[(3,5-bis-trifluoromethyl-benzyl)-(1-oxo-1,2-dihydroisoquinolin-3-ylmethyl)-amino]-pyrimidin-5-yl}-piperidine-4-carboxylate (100 mg) is dissolved in ethanol (1 ml) and thereto is added a 2N-aqueous sodium hydroxide solution (215 μl) and the mixture is stirred at room temperature overnight. To the reaction solution are added water and a 1N-hydrochloric acid to adjust the mixture to be pH 4, and the mixture is extracted with chloroform. The organic layer is dried over magnesium sulfate and the mixture is concentrated under reduced pressure to give 1-{2-[(3,5-bis-trifluoromethyl-benzyl)-(1-oxo-1,2-dihydroisoquinolin-3-ylmethyl)-amino]-pyrimidin-5-yl}-piperidine-4-carboxylic acid (85 mg). MS (m/z): 606 [M+H]⁺

TABLE 22

| | | Physical |
|---|---|---|
| Ex. | | properties, |
| No. | Structural formula | etc. |
| 66 | 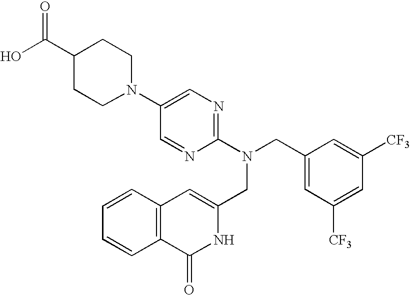 | MS (m/z):606[M + H]⁺ |

### Example 67

Ethyl 1-{2-[(1-benzyloxy-isoquinolin-3-ylmethyl)-(3,5-bis-trifluoromethyl-benzyl)-amino]-pyrimidin-5-yl}-piperidine-4-carboxylate (150 mg) is treated in a similar manner to Example 4(2) to give 1-{2-[(1-benzyloxy-isoquinolin-3-ylmethyl)-(3,5-bis-trifluoromethyl-benzyl)-amino]-pyrimidin-5-yl}-piperidine-4-carboxylic acid (127 mg). MS (m/z): 696 [M+H]⁺

### Example 68

(1) Ethyl 1-{2-[(3,5-bis-trifluoromethyl-benzyl)-(1-oxo-1,2-dihydroisoquinolin-3-ylmethyl)-amino]-pyrimidin-5-yl}-piperidine-4-carboxylate (300 mg), isopropanol (49 μl) and triphenylphosphine (170 mg) are dissolved in tetrahydrofuran (5 ml), and thereto is added dropwise a 40% diethyl azodicarboxylate/toluene (255 μl) and the mixture is stirred at room temperature for 1 hour. The reaction solution is concentrated under reduced pressure and the resulting residue is purified by NH-silica gel column chromatography (hexane:ethyl acetate=1:0→95:5) to give ethyl 1-{2-[(3,5-bis-trifluoromethyl-benzyl)-(1-isopropoxy-isoquinolin-3-ylmethyl)-amino]-pyrimidin-5-yl}-piperidine-4-carboxylate (281 mg). MS (m/z): 676 [M+H]⁺

(2) Ethyl 1-{2-[(3,5-bis-trifluoromethyl-benzyl)-(1-isopropoxy-isoquinolin-3-ylmethyl)-amino]-pyrimidin-5-yl}-piperidine-4-carboxylate (275 mg) is dissolved in ethanol (5 ml), and thereto is added a 2N-aqueous sodium hydroxide solution (610 μl) and the mixture is stirred at room temperature overnight. To the reaction solution are added water and a 1N-hydrochloric acid to adjust the mixture to be pH 4 and the mixture is extracted with chloroform. The organic layer is dried over magnesium sulfate and concentrated under reduced pressure. The resulting residue is purified by silica gel column chromatography (chloroform:methanol=1:0→94:6) to give 1-{2-[(3,5-bis-trifluoromethyl-benzyl)-(1-isopropoxy-isoquinolin-3-ylmethyl)-amino]-pyrimidin-5-yl}-piperidine-4-carboxylic acid (215 mg). MS (m/z): 648 [M+H]⁺

### Example 69

(1) Ethyl 1-{2-[(3,5-bis-trifluoromethyl-benzyl)-(1-oxo-1,2-dihydroisoquinolin-3-ylmethyl)-amino]-pyrimidin-5-yl}-piperidine-4-carboxylate (300 mg), 2-methoxyethanol (51 μl) and triphenylphosphine (170 mg) are dissolved in tetrahydrofuran (5 ml), and thereto is added dropwise a 40% diethyl azodicarboxylate/toluene (255 μl), and the mixture is stirred at room temperature for 1 hour. The reaction solution is concentrated under reduced pressure and the resulting residue is purified by NH-silica gel column chromatography (hexane:ethyl acetate=93:7→67:33) to give ethyl 1-(2-{(3,5-bis-trifluoromethyl-benzyl)-[1-(2-methoxy-ethoxy)-isoquinolin-3-ylmethyl]-amino}-pyrimidin-5-yl)-piperidine-4-carboxylate (187 mg; MS (m/z): 692 [M+H]⁺) and ethyl 1-(2-{(3,5-bis-trifluoromethyl-benzyl)-[2-(2-methoxy-ethyl)-1-oxo-1,2-dihydroisoquinolin-3-ylmethyl]-amino}-pyrimidin-5-yl)-piperidine-4-carboxylate (94 mg; MS (m/z): 692 [M+H]⁺).

(2) Ethyl 1-(2-{(3,5-bis-trifluoromethyl-benzyl)-[1-(2-methoxy-ethoxy)-isoquinolin-3-ylmethyl]-amino}-pyrimidin-5-yl)-piperidine-4-carboxylate obtained in the above (1) (180 mg) is dissolved in ethanol (3 ml), and thereto is added a 2N-aqueous sodium hydroxide solution (390 μl) and the mixture is stirred at room temperature overnight. To the reaction solution are added water and a 1N-hydrochloric acid to adjust the mixture to be pH 4 and the mixture is extracted with chloroform. The organic layer is dried over magnesium sulfate and concentrated under reduced pressure. The resulting residue is purified by silica gel column chromatography (chloroform:methanol=1:0→93:7) to give 1-(2-{(3,5-bis-trifluoromethyl-benzyl)-[1-(2-methoxy-ethoxy)-isoquinolin-3-ylmethyl]-amino}-pyrimidin-5-yl)-piperidine 4-carboxylic acid (147 mg). MS (m/z): 664 [M+H]⁺

### Examples 70 to 72

The corresponding starting compounds are treated in a similar manner to Example 69 to give the compounds as listed in Table 23.

TABLE 23

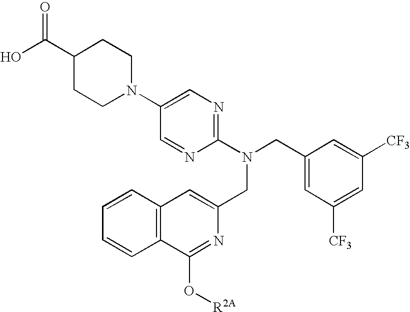

| | | Physical |
|---|---|---|
| Ex. | | properties, |
| No. | —R^{2A} | etc. |
| 67 | 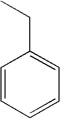 | MS (m/z):696[M + H]⁺ |
| 68 | 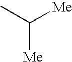 | MS (m/z):648[M + H]⁺ |
| 69 | 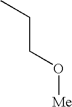 | MS (m/z):664[M + H]⁺ |
| 70 | 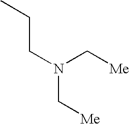 | MS (m/z):705[M + H]⁺ |
| 71 | 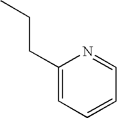 | MS (m/z):711[M + H]⁺ |
| 72 | 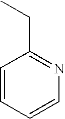 | MS (m/z):697[M + H]⁺ |

### Example 73

Ethyl 1-(2-{(3,5-bis-trifluoromethyl-benzyl)-[2-(2-methoxy-ethyl)-1-oxo-1,2-dihydroisoquinolin-3-ylmethyl]-amino}-pyrimidin-5-yl)-piperidine-4-carboxylate obtained in Example 69(1) (90 mg) is dissolved in ethanol (2 ml), and thereto is added a 2N-aqueous sodium hydroxide solution (195 μl) and the mixture is stirred at room temperature overnight. To the reaction solution are added water and a 1N-hydrochloric acid to adjust the mixture to be about pH 4, and the mixture is extracted with chloroform. The organic layer is dried over magnesium sulfate and concentrated under reduced pressure. The resulting residue is purified by silica gel column chromatography (chloroform:methanol=99:1→92:8) to give 1-(2-{(3,5-bis-trifluoromethyl-benzyl)-[2-(2-methoxy-ethyl)-1-oxo-1,2-dihydroisoquinolin-3-ylmethyl]-amino}-pyrimidin-5-yl)-piperidine-4-carboxylic acid (22 mg). MS (m/z): 664 [M+H]⁺

### Examples 74 to 76

The corresponding starting compounds are treated in a similar manner to Example 73 to give the compounds as listed in Table 24.

TABLE 24

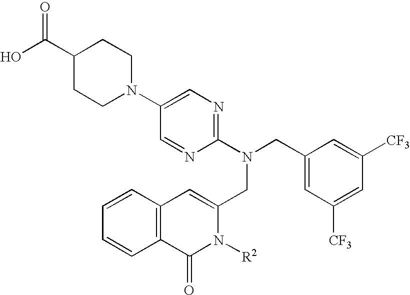

| | | Physical |
|---|---|---|
| Ex. | | properties, |
| No. | —R² | etc. |
| 73 | 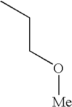 | MS (m/z):664[M + H]⁺ |
| 74 | 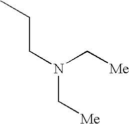 | MS (m/z):705[M + H]⁺ |
| 75 | 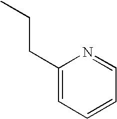 | MS (m/z):711[M + H]⁺ |
| 76 | 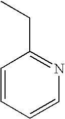 | MS (m/z):697[M + H]⁺ |

### Examples 77 to 78

The corresponding starting compounds are treated in similar manners to any of the above Examples to give the compounds as listed in Table 25.

TABLE 25

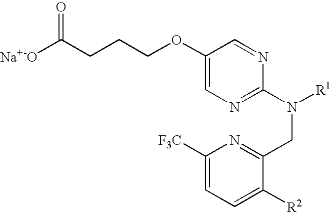

| | | | Physical |
|---|---|---|---|
| Ex. | | | properties, |
| No. | —R¹ | —R² | etc. |
| 77 | 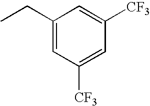 | 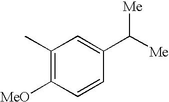 | |
| 78 | 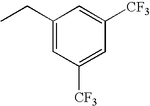 | 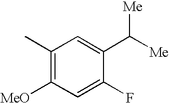 | |
| 79 | 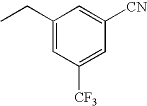 | 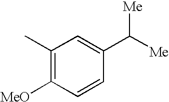 | |
| 80 | 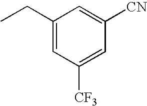 | 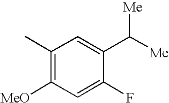 | |

### Examples 81 to 84

The corresponding starting compounds are treated in similar manners to any of the above Examples to give the compounds as listed in Table 26.

TABLE 26

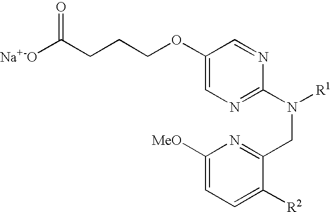

| | | | Physical |
|---|---|---|---|
| Ex. | | | properties, |
| No. | —R¹ | —R² | etc. |
| 81 | 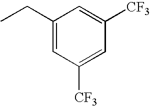 | 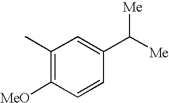 | |
| 82 | 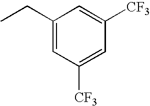 | 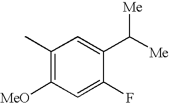 | |
| 83 | 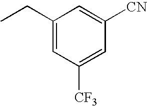 | 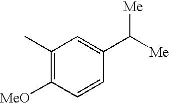 | |
| 84 | 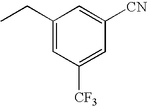 | 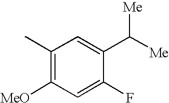 | |

### Reference Example 1

Cyclohexanecarboxaldehyde (38 g), diethylamine hydrochloride (55 g) and acetic acid (29 ml) are dissolved in methylene chloride (500 ml) and thereto is added triacetoxy sodium borohydride (71.8 g) at room temperature and the mixture is stirred at room temperature overnight. To the reaction solution are added a 2N-aqueous sodium hydroxide solution and methylene chloride, and the mixture is separated, and the organic layer is washed with a saturated brine, dried over magnesium sulfate, and the mixture is concentrated under reduced pressure to give cyclohexylmethyl-ethyl-amine (40.1 g) as a crude product. MS (m/z): 142 [M+H]⁺

### Reference Example 2

3,5-Bis-trifluoromethyl-benzylamine (10 g) and 5-bromo-2-chloro-pyrimidine (12 g) is dissolved in 1,4-dioxane (50 ml) and thereto is added N,N-diisopropylethylamine (10.7 ml) and the mixture is heated under reflux overnight. The reaction solution is cooled to room temperature and concentrated under reduced pressure. The resulting residue is purified by silica gel column chromatography (hexane:ethyl acetate=9:1→7:3) to give (3,5-bis-trifluoromethyl-benzyl)-(5-bromo-pyrimidin-2-yl)-amine (10.1 g). MS (m/z): 713 [M+H]⁺

### Reference Example 3

(1) Ethylamine hydrochloride (2 g) is dissolved in methylene chloride (20 ml) and thereto are added pyridine (6 ml) and ethyl 6-(chloroformyl)hexanoate (7.6 g) and the mixture is stirred at room temperature overnight. To the reaction solution are added a saturated aqueous sodium bicarbonate solution and the mixture is separated, and the organic layer is washed successively with a 1N-hydrochloric acid, water and a saturated brine, dried over magnesium sulfate, and concentrated under reduced pressure to give ethyl 6-ethylcarbamoyl hexanoate (9.29 g) as a crude product. MS (m/z): 216 [M+H]⁺

(2) Crude ethyl 6-ethylcarbamoyl hexanoate (9.29 g) is dissolved in tetrahydrofuran (50 ml) and thereto is added sodium borohydride (7.35 g). The reaction solution is heated under reflux and thereto is added dropwise acetic acid (11 ml) and the mixture is heated under reflux for 1 hour and 30 minutes. To the reaction solution is added water under ice-cooling and the mixture is extracted with ethyl acetate. The organic layer is washed with a saturated brine, dried over magnesium sulfate, and concentrated under reduced pressure. The resulting residue is dissolved in ethanol (30 ml), and thereto is added a 4N-hydrochloric acid in ethyl acetate (7.6 ml), and the mixture is stirred at room temperature overnight. To the reaction solution are added a 2N-aqueous sodium hydroxide solution and ethyl acetate and the mixture is separated, and the organic layer is washed with a saturated brine, dried over magnesium sulfate, and concentrated under reduced pressure to give ethyl 7-ethylamino-heptanoate (3.98 g) as a crude product. MS (m/z): 216 [M+H]⁺

### Reference Example 4

(1) 6-Aminohexanoic acid methyl ester hydrochloride (5 g) is dissolved in methylene chloride (20 ml) and thereto are added pyridine (4.5 ml) and acetyl chloride (2 ml), and the mixture is stirred at room temperature for 1 hour and 45 minutes. To the reaction solution are added a 1N-hydrochloric acid and chloroform, and the mixture is separated, and the organic layer is washed with a saturated brine, dried over magnesium sulfate, and concentrated under reduced pressure to give methyl 6-acetylamino-hexanoate (5.19 g) as a crude product. MS (m/z): 188 [M+H]⁺

(2) Crude methyl 6-acetylamino-hexanoate (5.19 g) is dissolved in tetrahydrofuran (50 ml) and thereto is added sodium borohydride (5.03 g). The reaction solution is heated under reflux and thereto is added dropwise acetic acid (7.6 ml) and the mixture is heated under reflux for 1 hour and 30 minutes. To the reaction solution are added water under ice-cooling and the mixture is extracted with ethyl acetate. The organic layer is washed with a saturated brine, dried over magnesium sulfate, and concentrated under reduced pressure. The resulting residue is dissolved in ethanol (30 ml) and thereto is added a 4N-hydrochloric acid in ethyl acetate (7.6 ml) and the mixture is stirred at room temperature overnight. To the reaction solution are added a 2N-aqueous sodium hydroxide solution and ethyl acetate, and the mixture is separated, and the organic layer is washed with a saturated brine, dried over magnesium sulfate, and concentrated under reduced pressure to give ethyl 6-ethylamino-hexanoate (1.74 g) as a crude product. MS (m/z): 188 [M+H]⁺

### Reference Example 5

(1) (3,5-Bis-trifluoromethyl-benzyl)-(5-bromopyrimidin-2-yl)-amine (10 g) and triethylamine (4.18 ml) are dissolved in methylene chloride (100 mL) and thereto is added triphosgene (2.97 g) under ice-cooling. The reaction solution is stirred at the same temperature for 30 minutes and concentrated under reduced pressure. The resulting residue is dissolved in tetrahydrofuran (100 mL) and thereto are added benzyl alcohol (3.88 ml) and triethylamine (10.45 ml) at room temperature, and the mixture is stirred overnight. The reaction solution is diluted with ethyl acetate and washed with a saturated aqueous sodium bicarbonate solution and a 1N-hydrochloric acid. The organic layer is washed with a saturated brine, dried over magnesium sulfate, and concentrated under reduced pressure. The resulting residue is purified by silica gel column chromatography (hexane:ethyl acetate=97:3→9:1) to give benzyl (3,5-bis-trifluoromethyl-benzyl)-(5-bromo-pyrimidin-2-yl)-carbamate (11.58 g). MS (m/z): 534/536 [M+H]⁺

(2) Benzyl (3,5-bis-trifluoromethyl-benzyl)-(5-bromo-pyrimidin-2-yl)-carbamate (11.5 g), [1,1′-bis(diphenylphosphino)ferrocene]-dichloropalladium dichloromethane complex (3.51 g), potassium acetate (6.33 g) and bis(pinacolato)diboron (10.9 g) are dissolved in dimethylsulfoxide (75 ml), and the mixture is heated to 80° C. under nitrogen atmosphere and stirred for 30 minutes. The reaction solution is cooled to room temperature and thereto are added water and ethyl acetate, and the insoluble materials are removed by filtration through Celite™, and the mixture is separated and the organic layer is washed with a saturated brine, dried over magnesium sulfate, and concentrated under reduced pressure. The resulting residue is dissolved in tetrahydrofuran (100 ml) and thereto is added dropwise a 30% aqueous hydrogen peroxide solution (50 ml) under ice-cooling and the mixture is stirred for 1 hour. Thereto is added a saturated aqueous sodium thiosulfate solution under ice-cooling to consume the excess hydrogen peroxide, followed by an addition of water and ethyl acetate, and the mixture is separated. The organic layer is washed with a saturated brine, dried over magnesium sulfate, and concentrated under reduced pressure. The resulting residue is purified by silica gel column chromatography (chloroform:methanol=1:0→9:1) to give benzyl (3,5-bis-trifluoromethyl-benzyl)-(5-hydroxy-pyrimidin-2-yl)-carbamate (9.70 g). MS (m/z): 472 [M+H]⁺

(3) Benzyl (3,5-bis-trifluoromethyl-benzyl)-(5-hydroxy-pyrimidin-2-yl)-carbamate (9.70 g) and ethyl 4-bromobutyrate (3.53 g) are dissolved in N,N-dimethylformamide (50 mL) and thereto is added potassium carbonate (3.41 g) and the mixture is stirred at 50° C. for 1 hour. Thereto are added ethyl acetate and a saturated brine, and the mixture is separated, and the organic layer is washed with a saturated brine, dried over magnesium sulfate, and concentrated under reduced pressure. The resulting residue is purified by silica gel column chromatography (hexane:ethyl acetate=4:1→1:1) to give ethyl 4-{2-[benzyloxycarbonyl-(3,5-bis-trifluoromethyl-benzyl)-amino]-pyrimidin-5-yloxy}-butyrate (8.29 g). MS (m/z): 586 [M+H]⁺

(4) Ethyl 4-{2-[benzyloxycarbonyl-(3,5-bis-trifluoromethyl-benzyl)-amino]-pyrimidin-5-yloxy}-butyrate (3.0 g) is dissolved in tetrahydrofuran (20 ml) and thereto is added 10% palladium-carbon (500 mg) and the mixture is stirred under hydrogen atmosphere at room temperature for 2 hours and 30 minutes. The catalyst is removed by filtration, and the filtrate is concentrated under reduced pressure. The resulting residue is purified by silica gel column chromatography (hexane:ethyl acetate=9:1→1:1) to give ethyl 4-[2-(3,5-bis-trifluoromethyl-benzylamino)-pyrimidin-5-yloxy]-butyrate (2.22 g). MS (m/z): 452 [M+H]⁺

### Reference Example 6

(1) 6-Aminohexanoic acid methyl ester hydrochloride (2.50 g) is dissolved in tetrahydrofuran (50 ml) and thereto are added water (50 ml) and sodium bicarbonate (3.44 g), followed by an addition dropwise of benzyl chloroformate (2.17 ml) under ice-cooling, and the mixture is stirred at the same temperature for 2 hours and a half. To the reaction mixture is added a saturated brine, and the mixture is extracted with ethyl acetate. The organic layer is washed twice with a saturated brine, dried over magnesium sulfate, and concentrated under reduced pressure to give methyl 6-benzyloxycarbonylamino hexanoate (4.16 g). MS (m/z): 280 [M+H]⁺

(2) Methyl 6-benzyloxycarbonylamino-hexanoate (4.15 g) is dissolved in N,N-dimethylformamide (25 mL) and thereto is added sodium hydride (63%) (552 mg) under ice-cooling and the mixture is stirred at the same temperature for 1 hour and thereto is added methyl iodide (1.72 ml), and the mixture is stirred at the same temperature for an additional 2 hours. To the reaction mixture is added a saturated brine, and the mixture is extracted with ethyl acetate, and the organic layer is washed with a saturated brine, dried over magnesium sulfate, and concentrated under reduced pressure. The resulting residue is purified by silica gel column chromatography (hexane:ethyl acetate=4:1) to give methyl 6-(benzyloxycarbonyl-methyl-amino)-hexanoate (2.25 g). MS (m/z): 294 [M+H]⁺

(3) Methyl 6-(benzyloxycarbonyl-methyl-amino)-hexanoate (2.24 g) is dissolved in methanol (35 ml) and thereto is added 10% palladium-carbon (500 mg) and the mixture is stirred at room temperature under hydrogen atmosphere for 2 hours. The reaction mixture is filtered and the filtrate is concentrated under reduced pressure to give methyl 6-methylamino hexanoate (1.11 g). MS (m/z): 160 [M+H]⁺

### Reference Example 7

Propylamine (0.65 g) is dissolved in tetrahydrofuran (5 ml) and thereto is added pyridine (0.89 ml), followed by an addition dropwise of methyl adipoyl chloride (1.96 g) under ice-cooling and the mixture is stirred at room temperature for 1 hour. To the reaction mixture is added a saturated brine, and the mixture is extracted with ethyl acetate. The organic layer is washed with a saturated brine, dried over magnesium sulfate, and concentrated under reduced pressure. The resulting residue is dissolved in tetrahydrofuran (30 ml) and thereto is added sodium borohydride (1.93 g) at room temperature and the mixture is heated to 65° C. and thereto is added dropwise acetic acid (2.92 ml) over 1 hour and the mixture is stirred at the same temperature for 9 hours. To the reaction mixture is added an ice-cooled dilute hydrochloric acid, and the mixture is stirred for 30 minutes and extracted with ethyl acetate, and the organic layer is washed with a mixed solution of a saturated aqueous sodium bicarbonate solution and a saturated brine, dried over magnesium sulfate, and concentrated under reduced pressure. The resulting residue is dissolved in methanol (15 ml) and thereto is added a 4N-hydrochloric acid in dioxane (7.5 mL) and the mixture is stirred at room temperature overnight. To reaction mixture is added a saturated aqueous sodium bicarbonate solution and a saturated brine, and the mixture is extracted six times with ethyl acetate and the collected organic layer is dried over magnesium sulfate, and concentrated under reduced pressure to give methyl 6-propylamino-hexanoate (914 mg). MS (m/z): 188 [M+H]⁺

### Reference Example 8

(1) Tert-butyl piperidine-4-ylmethyl-carbamate (2.00 g) is dissolved in tetrahydrofuran (10 ml), and thereto is added triethylamine (1.69 ml), followed by an addition dropwise of ethyl bromoacetate (1.24 mL) in water bath and the mixture is stirred at room temperature for 2 hours. To the reaction mixture is added a saturated brine, and the mixture is extracted with ethyl acetate. The organic layer is washed twice with a saturated brine, dried over magnesium sulfate, and concentrated under reduced pressure, and to the resulting crystalline residue is added isopropylether and the mixture is filtered to give ethyl [4-(tert-butoxy-carbonylamino-methyl)-piperidin-1-yl]-acetate (1.87 g). MS (m/z): 301 [M+H]⁺

(2) Ethyl [4-(tert-butoxycarbonylamino-methyl)-piperidin-1-yl]-acetate (1.86 g) is dissolved in N,N-dimethylformamide (10 ml), and thereto are added sodium hydride (63%) (1.19 g) and ethyl iodide (6.0 ml), and the mixture is stirred at room temperature for 2 hours. The reaction mixture is made weak basic with a 10% aqueous citric acid solution and a saturated aqueous sodium bicarbonate solution, and extracted with ethyl acetate, and the organic layer is washed with a saturated brine, dried over magnesium sulfate, and concentrated under reduced pressure. The resulting residue is purified by silica gel column chromatography (hexane:ethyl acetate=2:1) to give ethyl {4-[(tert-butoxycarbonyl-ethyl-amino)-methyl]-piperidine-1-yl}-acetate (905 mg). MS (m/z): 329 [M+H]⁺

(3) Ethyl {4-[(tert-butoxycarbonyl-ethyl-amino)-methyl]-piperidine-1-yl}-acetate (235 mg) is dissolved in methylene chloride (1 ml) and thereto is added trifluoroacetic acid (1 ml) and the mixture is stirred at room temperature overnight. The reaction mixture is concentrated under reduced pressure to give (4-ethylaminomethyl-piperidin-1-yl)-acetic acid ethyl ester bistrifluoroacetic acid salt (482 mg). MS (m/z): 229 [M+H]⁺

### Reference Example 9

(1) 2-Tert-butoxy-ethylamine (2 g) is dissolved in methylene chloride (10 ml) and thereto are added pyridine (940 μl) and ethyl 6-(chloroformyl)hexanoate (1.13 g) under ice-cooling and the mixture is stirred at room temperature overnight. To the reaction solution are added a 1N-hydrochloric acid and chloroform, and the mixture is separated, and the organic layer is washed with a saturated brine, dried over magnesium sulfate, and concentrated under reduced pressure to give ethyl 6-(2-tert-ethylcarbamoyl)-hexanoate as a crude product (2.96 g). MS (m/z): 288 [M+H]⁺

(2) Crude ethyl 6-(2-tert-ethylcarbamoyl)-hexanoate (2.96 g) is dissolved in tetrahydrofuran (15 ml) and thereto is added sodium borohydride (1.60 g). The reaction solution is heated under reflux and thereto is added dropwise acetic acid (2.4 mL) and the mixture is heated under reflux for 1 hour. To the reaction solution is added water under ice-cooling and the mixture is extracted with ethyl acetate. The organic layer is washed with a saturated brine, dried over magnesium sulfate, and concentrated under reduced pressure. The resulting residue is dissolved in ethanol (6 ml) and thereto is added a 4N-hydrochloric acid in ethyl acetate (1.5 ml) and the mixture is stirred at room temperature for 3 days. To the reaction solution are added a saturated aqueous sodium bicarbonate solution and ethyl acetate, and the mixture is separated, and the organic layer is washed with a saturated brine, dried over magnesium sulfate, and concentrated under reduced pressure to give ethyl 7-(2-tert-butoxy-ethylamino)-heptanoate as a crude product (2.6 g). MS (m/z): 274 [M+H]⁺

### Reference Example 10

(1) 3-Nitro-5-(trifluoromethyl)benzoic acid (50 g) is dissolved in tetrahydrofuran (300 ml) and thereto is added dropwise a 1.0M-borane tetrahydrofuran complex/tetrahydrofuran (300 ml) at 0° C. under nitrogen atmosphere over 2 hours and the mixture is stirred at 75° C. for 1 hour and a half. The reaction solution is allowed to cool to room temperature and concentrated under reduced pressure, and thereto is added a 1N-hydrochloric acid and the mixture is extracted with ethyl acetate. The organic layer is washed successively with water and a saturated brine, dried over magnesium sulfate, and concentrated under reduced pressure to give crude (3-nitro-5-trifluoromethyl-phenyl)-methanol. The obtained crude product is dissolved in methanol (500 mL) and thereto is added 10% palladium-carbon (5 g) and the mixture is stirred under hydrogen atmosphere at room temperature overnight. The catalyst is removed by filtration, and the filtrate is concentrated under reduced pressure to give crude (3-amino-5-trifluoromethyl-phenyl)-methanol. To copper (II) bromide (53.6 g) is added acetonitrile (500 ml), followed by an addition dropwise of tert-butyl nitrite (35.7 ml) under ice-cooling and the mixture is stirred under nitrogen atmosphere for 5 minutes. To reaction mixture is added dropwise a solution of the above crude (3-amino-5-trifluoromethyl-phenyl)-methanol in acetonitrile (200 ml) under ice-cooling over 1 hour and 15 minutes and the mixture is stirred at room temperature under nitrogen atmosphere overnight. To reaction mixture is added a 1N-hydrochloric acid and the mixture is extracted with ethyl acetate. The organic layer is washed successively with a 1N-hydrochloric acid, water and a saturated brine, dried over magnesium sulfate, and concentrated under reduced pressure. The resulting residue is purified by silica gel column chromatography (hexane:ethyl acetate=7:1→4:1) to give (3-bromo-5-trifluoromethyl-phenyl)-methanol (40.7 g). NMR (CDCl₃): 1.90 (1H, t), 4.76 (2H, d), 7.56 (1H, s), 7.68 (1H, s), 7.72 (1H, s)

(2) (3-Bromo-5-trifluoromethyl-phenyl)-methanol (33.9 g) is dissolved in N,N-dimethylformamide (400 mL) and thereto are added zinc(II) cyanide (16.39 g) and tetrakis(triphenylphosphine)palladium (7.68 g) and the mixture is heated under nitrogen atmosphere at 120° C. for 2 hours. The reaction solution is allowed to cool to room temperature, and filtered through Celite™, and the filtrate is concentrated under reduced pressure. Thereto is added water and the mixture is extracted with ethyl acetate. The organic layer is washed with a saturated brine, dried over sodium sulfate, and concentrated under reduced pressure. The resulting residue is purified by silica gel column chromatography (hexane:ethyl acetate=2:1) to give 3-hydroxymethyl-5-trifluoromethyl-benzonitrile (23.4 g). NMR (CDCl₃): 2.09 (1H, t), 4.85 (2H, d), 7.83 (1H, s), 7.87 (2H, s)

(3) 3-Hydroxymethyl-5-trifluoromethyl-benzonitrile (23.4 g) is dissolved in methylene chloride (230 mL) and thereto is added carbon tetrabromide (42.4 g), followed by an addition of triphenylphosphine (32.0 g) under ice-cooling and the mixture is stirred at the same temperature for 30 minutes. The reaction solution is concentrated under reduced pressure. The resulting residue is purified by silica gel column chromatography (hexane:ethyl acetate=10:1) to give 3-bromomethyl-5-trifluoromethyl-benzonitrile (25.5 g). NMR (CDCl₃): 4.51 (2H, s), 7.86 (1H, s), 7.88 (2H, s)

### Reference Example 11

(1) 2-Bromo-pyridin-3-ol (5 g) is dissolved in water (150 ml) and thereto are added sodium carbonate (6.15 g) and iodine (7.65 g) and the mixture is stirred at room temperature for 2 hours. Thereto are added a 1N-hydrochloric acid and ethyl acetate, and the mixture is separated, and the organic layer is washed with a saturated brine, dried over magnesium sulfate, and concentrated under reduced pressure. The residue is purified by silica gel column chromatography (hexane:ethyl acetate=9:1→3:1) to give 2-bromo-6-iodo-pyridin-3-ol (4.52 g). MS (m/z): 300/302 [M+H]⁺

(2) 2-Bromo-6-iodo-pyridin-3-ol (2.98 g) is dissolved in N,N-dimethylformamide (140 ml) and thereto are added cesium carbonate (16.3 g) and methyl iodide (1.25 mL) and the mixture is stirred at room temperature overnight. To the reaction solution are added water and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=19:14→:1) to give 2-bromo-6-iodo-3-methoxy-pyridine (2.45 g). MS (m/z): 314/316 [M+H]⁺

(3) To 0.5M-isopropenyl magnesium bromide/tetrahydrofuran is added trimethyl borate (3.3 ml) and the mixture is stirred under nitrogen atmosphere at room temperature for 30 minutes. To the reaction solution are added 6N-hydrochloric acid and diethylether, and the mixture is separated. The organic layer is washed with a saturated brine, dried over magnesium sulfate, and concentrated under reduced pressure to give isopropenylboronic acid as a crude product (818 mg). The above crude isopropenylboronic acid (287 mg) and 2-bromo-6-iodo-3-methoxy-pyridine (800 mg) are dissolved in a mixed solvent of 1,2-dimethoxy-ethane (8 ml) and ethanol (3.2 ml) and thereto are added a 1M-aqueous sodium carbonate solution (6.4 ml) and tetrakis(triphenylphosphine)palladium (240 mg) and the mixture is stirred under nitrogen atmosphere at 80° C. for 5 hours. To the reaction solution are added water and ethyl acetate, and the mixture is separated, and the organic layer is washed with a saturated brine, dried over magnesium sulfate, and concentrated under reduced pressure. The residue is purified by silica gel column chromatography (hexane:ethyl acetate=49:1→9:1) and followed by NH-silica gel column chromatography (hexane:ethyl acetate=49:1→9:1) to give 2-bromo-6-isopropenyl-3-methoxy-pyridine (138 mg). MS (m/z): 228/230 [M+H]⁺

### Reference Example 12

2-Bromo-6-iodo-3-methoxy-pyridine (500 mg) is dissolved in dry toluene (5 ml) and thereto is added dropwise 1.6M n-butyllithium in hexanes (1 ml) at −78° C. under nitrogen atmosphere, and the mixture is stirred for 1 hour and thereto is added acetone (0.23 ml), and the mixture is stirred overnight. To the reaction solution are added water and ethyl acetate, and the mixture is separated, and the organic layer is washed with a saturated brine, dried over magnesium sulfate, and concentrated under reduced pressure. The resulting residue is purified by silica gel column chromatography (hexane:ethyl acetate=4:1→3:2) to give 2-(6-bromo-5-methoxy-pyridin-2-yl)-propan-2-ol (197 mg). MS (m/z): 246/248 [M+H]⁺

### Reference Example 13

5-Methoxy-2-methylsulfanyl-pyrimidin-4-ol (250 mg) is dissolved in acetonitrile (7 ml) and thereto are added phosphorus oxychloride (0.7 ml) and N,N-diethyl aniline (460 μl) and the mixture is heated under reflux for 5.5 hours. The reaction solution is evaporated azeotropically with toluene three times, and to the residue are added aqueous citric acid solution and chloroform, and the mixture is separated. The organic layer is washed with a saturated brine, dried over magnesium sulfate, and concentrated under reduced pressure. The resulting residue is purified by silica gel column chromatography (hexane:ethyl acetate=19:1→17:3) to give 4-chloro-5-methoxy-2-methylsulfanyl-pyrimidine (260 mg). MS (m/z): 191/193 [M+H]⁺

### Reference Example 14

2-Chloropyrimidin-5-ol (3.89 g) is dissolved in N,N-dimethylformamide (50 ml) and thereto are added potassium carbonate (4.98 g) and tert-butyl 4-bromo-butyrate (7.36 g) and the mixture is stirred at room temperature overnight. To the reaction solution are added ethyl acetate and water, and the mixture is separated, and the organic layer is washed with a saturated brine, dried over magnesium sulfate, and concentrated under reduced pressure. The resulting residue is purified by silica gel column chromatography (hexane:ethyl acetate=24:1→4:1) to give tert-butyl 4-(2-chloropyrimidin-5-yloxy)bromobutyrate (6.22 g). MS (m/z): 273 [M+H]⁺

### Reference Example 15

2,5-Dibromopyridine (4.74 g) is dissolved in toluene (100 ml) and thereto are added 3,5-bis-trifluoromethyl-benzylamine (5.84 g) and palladium acetate (449.0 mg), 2,2′-bis(diphenylphosphino)-1,1′-binaphthyl (1.25 g) and sodium tert-butoxide (4.23 g) and the mixture is stirred under nitrogen atmosphere at 80° C. for 12 hours. The reaction solution is cooled to room temperature, and thereto is added a saturated aqueous sodium bicarbonate solution and the mixture is extracted with ethyl acetate twice, and the organic layer is washed successively with water and a saturated brine, dried over magnesium sulfate, and concentrated under reduced pressure. The resulting residue is purified by silica gel column chromatography (hexane:ethyl acetate=19:14→:1) to give (3,5-bis-trifluoromethyl-benzyl)-(5-bromopyridin-2-yl)-amine (2.08 g). MS (m/z): 399/401 [M+H]⁺

### Reference Example 16

3-Bromomethyl-5-trifluoromethyl-benzonitrile (which is prepared in Reference Example 12) (15.9 g) is dissolved in 7M-ammonia/methanol (550 ml), and the mixture is stirred at 50-60° C. for 30 minutes. The reaction solution is concentrated under reduced pressure. To the resulting residue are added a saturated aqueous sodium bicarbonate solution and chloroform, and the mixture is separated, and the organic layer is dried over sodium sulfate, and concentrated under reduced pressure. The resulting residue is purified by silica gel column chromatography (chloroform:methanol=1:0→19:1 chloroform:methanol: ammonium hydroxide solution=19:1:0.1) to give 3-aminomethyl-5-trifluoromethyl-benzonitrile (10.4 g). MS (m/z): 201 [M+H]⁺

### Reference Example 17

Tert-butyl 4-(2-chloropyrimidin-5-yloxy)-butyrate (5.0 g) is dissolved in toluene (100 ml) and thereto are added palladium acetate (412 mg) and 2,2′-bis(diphenylphosphino)-1,1′-binaphthyl (1.26 g), and the mixture is stirred under nitrogen atmosphere at 50° C. for 1 hour. The reaction solution is cooled to room temperature, and thereto are added 3,5-bis-trifluoromethyl-benzylamine (5.35 g) and sodium tert-butoxide (3.88 g) and the mixture is stirred at 35° C. for 2 hours. Thereto are added ethyl acetate and water, and the mixture is separated, and the organic layer is washed with a saturated brine, dried over magnesium sulfate, and concentrated under reduced pressure. The residue is purified by silica gel column chromatography (hexane:ethyl acetate=9:1→2:1) and NH-silica gel column chromatography (hexane:ethyl acetate=4:1→2:1) to give tert-butyl 4-[2-(3,5-bis-trifluoromethyl-benzylamino)-pyrimidin-5-yloxy]-butyrate (5.59 g). MS (m/z): 480 [M+H]⁺

### Reference Example 18

The corresponding starting compound is treated in a similar manner to Reference Example 17 to give the compound listed in the following Table 27.

### Reference Example 19

(1) 3-Bromo-4-methoxybenzoic acid (2.00 g) is dissolved in tetrahydrofuran (50 ml), and the mixture is cooled to −78° C., and thereto is added dropwise a 1.1M methyllithium in diethyl ether (7.7 ml). The mixture is stirred at −78° C. for 5 minutes, and thereto is added dropwise a 1.6M tert-butyllithium in n-pentane (13.2 ml), and the mixture is stirred at −78° C. for 15 minutes, and the mixture is allowed to warm to −45° C., and the mixture is stirred for 45 minutes, and then again cooled to −78° C. Thereto is added dropwise triisopropyl borate, and the mixture is stirred at −78° C. for 15 minutes, and the mixture is allowed to warm to room temperature. The mixture is stirred at room temperature for 1.5 hours, and the mixture is concentrated under reduced pressure, and thereto are added water and hexane. The aqueous layer is adjusted pH to 4 by addition of a 6N-hydrochloric acid and a saturated aqueous sodium bicarbonate solution, and the mixture is extracted with ethyl acetate and methanol twice. The organic layer is washed with a saturated brine, dried over magnesium sulfate and concentrated under reduced pressure. To the resulting residue is added diethyl ether, and the resulting solids are collected by filteration to give 2-methoxy-5-carboxyphenylboronic acid (1.37 g) as a crude product.

(2) The crude 2-methoxy-5-carboxyphenylboronic acid (370 mg) is dissolved in N,N-dimethylformamide (10 ml), and thereto are added 2.0M-dimethylamine/tetrahydrofuran solution (1.9 ml), 1-hydroxybenzotriazole dihydrate (725 mg), 1-ethyl-3-(3-dimethylaminopropyl)carbodiimide hydrochloride (579 mg). The mixture is stirred at room temperature for 3 hours, and thereto is added a saturated aqueous sodium bicarbonate solution, and the mixture is extracted with ethyl acetate twice. The organic layer is dried over magnesium sulfate and concentrated under reduced pressure. The resulting residue is purified by silica gel column chromatography (chloroform:methanol=19:1) to give 2-methoxy-5-dimethylcarbamoylphenylboronic acid (210 mg). MS (m/z): 224 [M+H]⁺

### Reference Example 20

The corresponding starting compound is treated in a similar manner to Reference Example 19 to give the compound listed in the following Table 27.

### Reference Example 21

(1) 4-sec-Butyl-phenol (3.0 g) is dissolved in chloroform and thereto is added bromine (1.02 ml) and the mixture is stirred at room temperature for 30 minutes. Thereto are added a saturated aqueous sodium thiosulfate solution, a saturated aqueous sodium bicarbonate solution and ethyl acetate, and the mixture is separated. The organic layer is washed with a saturated brine, dried over magnesium sulfate, and concentrated under reduced pressure to give 2-bromo-4-sec-butyl-phenol (4.57 g). NMR (CDCl₃): 0.81 (3H, t), 1.19 (3H, d), 1.56 (2H, m), 2.51 (1H, m), 5.33 (1H, s), 6.93 (1H, d), 7.02 (1H, d), 7.26 (1H, s).

(2) 2-Bromo-4-sec-butyl-phenol (1.50 g) is dissolved in N,N-dimethylformamide (10 ml) and thereto is added potassium carbonate (1.18 g) and iodomethane (1.12 g) and the mixture is stirred at room temperature overnight. Thereto are added ethyl acetate and water, and the mixture is separated, and the organic layer is washed with a saturated brine, dried over magnesium sulfate and concentrated under reduced pressure. The resulting residue is purified by silica gel column chromatography (hexane:ethyl acetate=1:0→20:1) to give 2-bromo-4-sec-butyl-1-methoxy-benzene (1.58 g). NMR (CDCl₃): 0.80 (3H, t), 1.20 (3H, d), 1.55 (2H, m), 2.51 (1H, m), 3.87 (3H, s), 6.82 (1H, d), 7.07 (1H, d), 7.35 (1H, s).

(3) 2-Bromo-4-sec-butyl-1-methoxy-benzene (1.15 g) is dissolved in tetrahydrofuran (17 ml) and the mixture is cooled to −78° C., and thereto is added dropwise 1.6M n-butyllithium in hexane, and the mixture is stirred at −78° C. for 15 minutes. To the reaction solution was added trimethyl borate (1.47 g), and the reaction mixture is stirred at −78° C. for 30 minutes, and thereto are added a saturated aqueous ammonium chloride solution and ethyl acetate. The mixture is separated, and the organic layer is washed with a saturated brine, dried over magnesium sulfate, and concentrated under reduced pressure to give crude 5-sec-butyl-1-methoxy-benzene boronic acid (950 mg). NMR (CDCl₃): 0.81 (3H, t), 1.22 (3H, d), 1.58 (2H, m), 2.58 (1H, m), 3.90 (3H, s), 6.25 (2H, s), 6.85 (2H, d), 7.25 (1H, d), 7.65 (1H, s).

### Reference Examples 22 to 25

The corresponding starting compounds are treated in a similar manner to Reference Example 21 to give the compounds listed in the following Table 27.

TABLE 27

| Ex. | |
|---|---|
| No. | Structural formura |
| 1 | 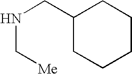 |
| 2 | 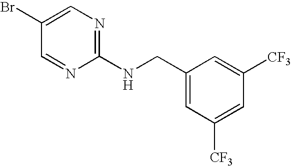 |
| 3 | 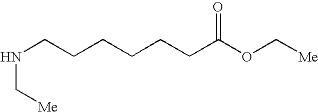 |
| 4 | 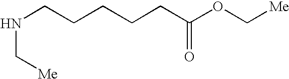 |
| 5 | 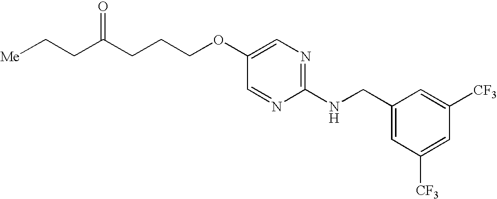 |
| 6 | 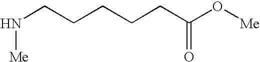 |
| 7 | 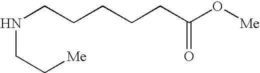 |
| 8 | 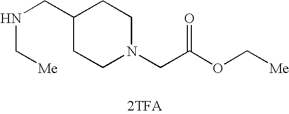 |

### INDUSTRIAL APPLICABILITY

The present compound of formula (1) or a pharmaceutically acceptable derivative thereof has an inhibitory activity against CETP and also shows an activity of increasing HDL cholesterol level and an activity of decreasing LDL cholesterol level. Thus, the compounds of the present invention are useful for prophylaxis and/or treatment of arteriosclerotic diseases, hyperlipemia or dyslipidemia, and the like.

1. A compound of the general formula (1): 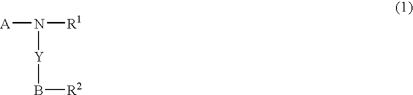
wherein, Y is a methylene group optionally substituted by a substituent(s) selected from an alkyl group and an oxo group, or a single bond;
A is (i) a group selected from an optionally substituted alkynyl group, a halogen atom, an oxo group, a hydroxy group, a cyano group, a nitro group, a carboxyl group, a sulfo group, an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, and a carbonyl group substituted by optionally substituted heterocyclic group;
(ii) a homocyclic group optionally substituted by 1 to 5 substituents selected independently from the groups as defined above in (i); or
(iii) a heterocyclic group optionally substituted by 1 to 5 substituents selected independently from the groups as defined above in (i);
B is a heterocyclic group optionally substituted by 1 to 5 substituents selected independently from the following groups: an optionally substituted alkynyl group, a halogen atom, an oxo group, a hydroxy group, a cyano group, a nitro group, a carboxyl group, a sulfo group, an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, a carbonyl group substituted by optionally substituted heterocyclic group, and an alkylene group; wherein said alkylene group may have 1 to 3 heteroatoms selected independently from oxygen, sulfur and nitrogen atoms and further optionally may have a substituent(s);
R¹ is a hydrogen atom or an optionally substituted alkyl group; wherein the alkyl group further may optionally be substituted by a substituent(s) selected from an optionally substituted homocyclic group and an optionally substituted heterocyclic group;
R² is a group selected from an optionally substituted alkynyl group, a halogen atom, an oxo group, a hydroxy group, a cyano group, a nitro group, a carboxyl group, a sulfo group, an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, and a carbonyl group substituted by optionally substituted heterocyclic group,
or a pharmaceutically acceptable derivative thereof.

2. The compound of claim 1 wherein the homocyclic group is a cycloalkyl group, a phenyl group or a naphthyl group;
the heterocyclic group is a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl or tetrahydroquinolyl group;
a substituent(s) for an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, a carbonyl group substituted by optionally substituted heterocyclic group, an optionally substituted phenyl group, an optionally substituted alkylsulfonyloxy group, an optionally substituted alkynyl group or an optionally substituted alkylene group is/are 1 to 5 groups selected independently from the following groups:
a halogen atom; a cyano group; a hydroxy group; a nitro group; a carboxyl group; an oxo group; a thioxo group; a sulfo group; a cycloalkyl group optionally substituted by hydroxy group, halogen atom, carboxyl group, alkoxycarbonyl group, mono- or di-alkylamino group, phenyl group or morpholinyl group; an alkoxycarbonyl group optionally substituted by hydroxy group, halogen atom, carboxyl group, alkoxycarbonyl group, mono- or di-alkylamino group, phenyl group or morpholinyl group; a carbamoyl group; a mono- or di-alkylcarbamoyl group optionally substituted by hydroxy group, halogen atom, carboxyl group, alkoxycarbonyl group, mono- or di-alkylamino group, phenyl group or morpholinyl group; an alkyl group optionally substituted by hydroxy group, halogen atom, carboxyl group, alkoxycarbonyl group, mono- or di-alkylamino group, phenyl group or morpholinyl group; an alkanoyl group optionally substituted by hydroxy group, halogen atom, carboxyl group, alkoxycarbonyl group, mono- or di-alkylamino group, phenyl group or morpholinyl group; an alkoxy group optionally substituted by hydroxy group, halogen atom, carboxyl group, alkoxycarbonyl group, mono- or di-alkylamino group, phenyl group or morpholinyl group; an alkanoyloxy group optionally substituted by hydroxy group, halogen atom, carboxyl group, alkoxycarbonyl group, mono- or di-alkylamino group, phenyl group or morpholinyl group; an alkylsulfanyl group optionally substituted by hydroxy group, halogen atom, carboxyl group, alkoxycarbonyl group, mono- or di-alkylamino group, phenyl group or morpholinyl group; an alkylsulfonyl group optionally substituted by hydroxy group, halogen atom, carboxyl group, alkoxycarbonyl group, mono- or di-alkylamino group, phenyl group or morpholinyl group; an alkylsulfinyl group optionally substituted by hydroxy group, halogen atom, carboxyl group, alkoxycarbonyl group, mono- or di-alkylamino group, phenyl group or morpholinyl group; a mono- or di-alkylsulfamoyl group optionally substituted by hydroxy group, halogen atom, carboxyl group, alkoxycarbonyl group, mono- or di-alkylamino group, phenyl group or morpholinyl group; an amino group; a mono- or di-alkylamino group optionally substituted by hydroxy group, halogen atom, carboxyl group, alkoxycarbonyl group, mono- or di-alkylamino group, phenyl group or morpholinyl group; a mono- or di-alkylsulfamoylamino group optionally substituted by hydroxy group, halogen atom, carboxyl group, alkoxycarbonyl group, mono- or di-alkylamino group, phenyl group or morpholinyl group; a mono- or di-alkylureido group optionally substituted by hydroxy group, halogen atom, carboxyl group, alkoxycarbonyl group, mono- or di-alkylamino group, phenyl group or morpholinyl group; a homocyclic group selected from a cycloalkyl group, a phenyl group and a naphthyl group; an oxy group substituted by the homocyclic group as defined above; a carbonyl group substituted by the homocyclic group as defined above; a heterocyclic group selected from a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl and tetrahydroquinolyl groups; an oxy group substituted by the heterocyclic group as defined above; a carbonyl group substituted by the heterocyclic group as defined above; and a group of the formulae: 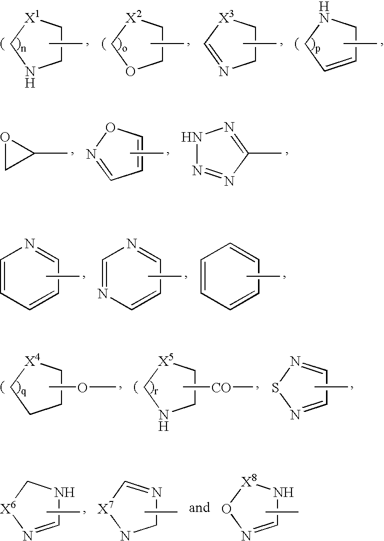
wherein X¹ and X³ are each independently CH₂, NH, O, S, SO or SO₂; X² and X⁵ are each independently CH₂, O, S, SO or SO₂; X⁴ is NH, O, S, SO or SO₂; X⁶ and X⁷ are each independently O or S; X⁸ is S or SO; and n, o, p, q and r are each independently an integer of 1 to 4, and further each of the above groups may optionally be substituted by 1 to 3 substituents selected from the following groups: halogen atom, carboxyl group, hydroxy group, cyano group, oxo group, thioxo group, alkyl group, hydroxyalkyl group, alkoxycarbonylalkyl group, carboxyalkyl group, morpholinylalkyl group, phenylalkyl group, alkanoyl group, hydroxyalkanoyl group, alkoxyalkanoyl group, alkoxy group, phenylalkoxy group, alkoxycarbonyl group, benzyloxycarbonyl group, mono- or di-alkylamino group, mono- or di-alkylcarbamoyl group, mono- or di-alkylsulfamoyl group, alkylsulfonyl group and tetrazolyl group,
or a pharmaceutically acceptable derivative thereof.

3. The compound of claim 2 wherein A is a group of a formula:
-A¹-A²;
wherein A¹ is a phenyl, naphthyl, pyrimidinyl, pyridazinyl, pyridyl, triazolyl, tetrazolyl, oxadiazolyl, dihydropyrimidinyl, pyrazinyl, thiazolyl, oxazolyl, dihydrooxazinyl, imidazolyl, pyrazolyl or dihydropyrazinyl group;
A² is a carboxyl group; a cyano group; a nitro group; an alkyl group optionally substituted by a group selected from a hydroxy group, a cyano group, a carboxyl group, an alkoxycarbonyl group, an alkoxy group, a phenylalkoxy group, a hydroxyalkoxy group, a carboxyalkoxy group, an alkylsulfanyl group, an alkylsulfonyl group, an alkylsulfinyl group, an amino group, a mono- or di-alkylamino group, a mono- or di-alkylsulfamoylamino group, a mono- or di-alkylureido group optionally substituted by morpholinyl group, an oxiranyl group, a dialkyldioxolanyl group, a pyrrolidinyl group optionally substituted by carboxyl group, a piperidyl group optionally substituted by carboxyl group, a piperazinyl group optionally substituted by alkyl group, and a morpholinyl group; an alkenyl group optionally substituted by carboxyl group; an alkoxy group optionally substituted by a group selected from a hydroxy group, a cyano group, a carboxyl group, an alkoxycarbonyl group, an alkoxy group, a phenylalkoxy group, a hydroxyalkoxy group, a carboxyalkoxy group, an alkylsulfanyl group, an alkylsulfonyl group, an alkylsulfinyl group, an amino group, a mono- or di-alkylamino group, a mono- or di-alkylsulfamoylamino group, a mono- or di-alkylureido group optionally substituted by morpholinyl group, an oxiranyl group, a dialkyldioxolanyl group, a pyrrolidinyl group optionally substituted by carboxyl group, a piperidyl group optionally substituted by carboxyl group, a piperazinyl group optionally substituted by alkyl group, and a morpholinyl group; an alkoxycarbonyl group; a hydroxycarbamimidoyl group; an alkylsulfanyl group; an alkylsulfonyl group optionally substituted by carboxyl group; a mono- or di-alkylamino group optionally substituted by hydroxy group, carboxyl group, alkoxy group or mono- or di-alkylamino group; a morpholinyl group optionally substituted by carboxyl group, alkyl group, carboxyalkyl group or alkoxycarbonyl group; an optionally oxidized thiomorpholinyl group; a piperazinyl group optionally substituted by a group selected from an alkyl group, alkanoyl group and hydroxyalkanoyl group; a pyrrolidinyl group optionally substituted by carboxyl group, alkyl group, carboxyalkyl group or alkoxycarbonyl group; a piperidyl group optionally substituted by carboxyl group, alkyl group, carboxyalkyl group or alkoxycarbonyl group; a tetrazolyl group optionally substituted by alkyl group, hydroxyalkyl group, carboxyalkyl group or morpholinylalkyl group; an oxodihydrooxadiazolyl group; a pyrimidinyl group; or a tetrahydropyranyl group;
R¹ is a group of a formula:
—R¹¹—R¹²,
wherein R¹¹ is an alkylene group;
R¹² is a substituent(s) selected from a phenyl, pyridyl, pyrimidinyl, pyridazinyl, furyl, thienyl, triazolyl, tetrazolyl, oxadiazolyl, dihydropyrimidinyl, pyrazinyl, thiazolyl, oxazolyl, imidazolyl, dihydrooxazinyl, dihydropyrazinyl and pyrazolyl group;
wherein said substituent(s) may optionally be substituted by 1 to 4 substituents selected independently from halogen atom, carboxyl group, alkoxycarbonyl group, carbamoyl group, mono- or di-alkylcarbamoyl group, alkyl group, alkoxy group, hydroxy group, nitro group, cyano group, amino group, mono- or di-alkylamino group, alkanoyl group, alkylsulfanyl group, tetrazolyl group and dihydrooxazolyl group; and further each of said alkyl group, alkoxy group, mono- or di-alkylamino group, mono- or di-alkylcarbamoyl group, alkanoyl group and alkylsulfanyl group independently may optionally be substituted by 1 to 5 substituents selected independently from halogen atom, hydroxy group, alkoxy group, amino group, morpholinyl group, piperidyl group, pyrrolidinyl group, piperazinyl group, alkylpiperazinyl group and alkanoylpiperazinyl group;
R² is a halogen atom;
a hydroxy group;
a cyano group;
a nitro group;
a carboxyl group;
a sulfo group;
a cycloalkyl group optionally substituted by carboxyl group or alkoxycarbonyl group;
an alkyl group optionally substituted by a group selected from a halogen atom, a cyano group, a hydroxy group, a carboxyl group, an alkoxycarbonyl group, a tetrazolyl group, a mono- or di-alkylcarbamoyl group, an alkoxy group (said alkoxy group may optionally be substituted by phenyl group, carboxyl group or hydroxy group), an alkanoyl group, an alkanoyloxy group, an alkylsulfanyl group, an alkylsulfonyl group, an alkylsulfinyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, an oxiranyl group, a dioxolanyl group optionally substituted by alkyl group, pyrrolidinyl group optionally substituted by alkoxycarbonyl group or carboxyl group, a pyrrolidinyl group optionally substituted by alkoxycarbonylalkyl group or carboxyalkyl group, a piperidyl group optionally substituted by alkoxycarbonyl group or carboxyl group, a piperidyl group optionally substituted by alkoxycarbonylalkyl or carboxyalkyl group, a piperazinyl group optionally substituted by alkyl group, a hexahydroazepinyl group, a morpholinyl group, and a piperidyloxy group optionally substituted by alkyl group;
an alkenyl group optionally substituted by a group selected from a cyano group, a hydroxy group, a carboxyl group, a benzyloxycarbonyl group and a tetrazolyl group;
an alkenyloxy group optionally substituted by carboxyl group;
an alkoxy group optionally substituted by a group selected from a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group optionally substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;
an alkoxycarbonyl group optionally substituted by phenyl group;
a carbamoyl group;
a mono- or di-alkylcarbamoyl group optionally substituted by a group selected from a carboxyl group, a morpholinyl group and an alkoxy group;
a hydroxycarbamimidoyl group;
an alkylsulfanyl group optionally substituted by a group selected from hydroxy group, carboxyl group and mono- or di-alkylcarbamoyl group;
an alkylsulfinyl group;
an alkylsulfonyl group optionally substituted by a group selected from hydroxy group, carboxyl group, alkoxycarbonyl group and mono- or di-alkylcarbamoyl group;
an amino group;
a mono- or di-alkylamino group optionally substituted by a group selected from a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl or carboxyalkyl group, a pyrrolidinyl group substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl optionally substituted by oxo group, a pyrrolidinylcarbonyl group optionally substituted by carboxyl group, a piperidyloxy group optionally substituted by alkyl group and a morpholinylcarbonyl group;
an alkanoylamino group optionally substituted by a group selected from hydroxy group, alkoxy group, carboxyl group and amino group;
a mono- or di-alkylcarbamoylamino group optionally substituted by alkoxy group;
a morpholinylcarbonylamino group;
a sulfamoyl group;
a mono- or di-alkylsulfamoyl group;
an alkanoyl group optionally substituted by a group selected from hydroxy group, carboxyl group, alkoxycarbonyl group, alkoxy group, mono- or di-alkylamino group and morpholinyl group; or
a cyclic group selected from a cycloalkyl, phenyl, naphthyl, thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl and tetrahydroquinolyl groups;
wherein said cyclic group may optionally be substituted by the following groups: a halogen atom, an alkoxyalkyl group, an alkyl group optionally substituted by 1 to 5 halogen atoms, a mono- or di-alkylaminoalkyl group, a mono- or di-alkylaminoalkoxy group, a carboxyl group, a hydroxy group, a cyano group, an oxo group, an alkyl group, a hydroxyalkyl group, an alkoxycarbonylalkyl group, a carboxyalkyl group, a morpholinylalkyl group, a phenylalkyl group, an alkanoyl group, a hydroxyalkanoyl group, an alkoxyalkanoyl group, an alkoxy group, a phenylalkoxy group, an alkoxycarbonyl group, a benzyloxycarbonyl group, a mono- or di-alkylamino group, a mono- or di-alkylcarbamoyl group, a mono- or di-alkylsulfamoyl group, an alkylsulfonyl group and a tetrazolyl group;
wherein the substituents defined as above may further be substituted by a substituent(s) selected from the following groups:
a halogen atom, an alkoxyalkyl group, an alkyl group optionally substituted by 1 to 5 halogen atoms, a mono- or di-alkylaminoalkyl group, a mono- or di-alkylaminoalkoxy group, a carboxyl group, a hydroxy group, a cyano group, an oxo group, an alkyl group, a hydroxyalkyl group, an alkoxycarbonylalkyl group, a carboxyalkyl group, a morpholinylalkyl group, a phenylalkyl group, an alkanoyl group, a hydroxyalkanoyl group, an alkoxyalkanoyl group, an alkoxy group, a phenylalkoxy group, an alkoxycarbonyl group, a benzyloxycarbonyl group, a mono- or di-alkylamino group, a mono- or di-alkylcarbamoyl group, a mono- or di-alkylsulfamoyl group, an alkylsulfonyl group and a tetrazolyl group,
or a pharmaceutically acceptable derivative thereof.

4. The compound of claim 1 wherein Y is a methylene group optionally substituted by a group(s) selected from an alkyl group and an oxo group, or a single bond;
A is a group of a formula:
-A¹-A²;
wherein A¹ is a homocyclic group, a heterocyclic group or a single bond;
A² is an optionally substituted homocyclic group, an optionally substituted alkylsulfanyl group, an optionally substituted alkylsulfinyl group, an optionally substituted alkylsulfonyl group, a nitro group, a hydroxy group, an optionally substituted alkenyl group, an optionally substituted heterocyclic group, an optionally substituted alkoxy group, a halogen atom, an optionally substituted alkyl group, a cyano group, an amino group optionally substituted by 1 to 2 substituents, or a hydrogen atom;
B is a heterocyclic group optionally substituted by 1 to 5 groups selected independently from an oxo group, a cyano group, a carboxyl group, an optionally substituted alkoxycarbonyl group, an optionally substituted carbamoyl group, a hydroxyl group, an optionally substituted alkylsulfonyl group, an optionally substituted alkyl group, a halogen atom, an optionally substituted alkoxy group, an optionally substituted alkylsulfanyl group, an optionally substituted cycloalkyl group, an optionally substituted cycloalkoxy group, an optionally substituted heterocyclic group, an optionally substituted alkylsulfinyl group and an amino group optionally substituted by 1 to 2 substituents;
R¹ is a hydrogen atom or an optionally substituted lower alkyl group; said lower alkyl group further may optionally be substituted by an optionally substituted homocyclic group or an optionally substituted heterocyclic group;
R² is an optionally substituted alkylsulfanyl group, an optionally substituted alkylsulfinyl group, an optionally substituted alkylsulfonyl group, a cyano group, an optionally substituted alkenyl group, an amino group optionally substituted by 1 to 2 substituents, a halogen atom, an optionally substituted alkoxy group, an optionally substituted carbamoyl group, an oxy group substituted by optionally substituted heterocyclic group, a hydroxy group, an optionally substituted heterocyclic group, an optionally substituted homocyclic group, an oxy group substituted by optionally substituted homocyclic group, an optionally substituted alkyl group, a hydrogen atom, an optionally substituted alkylcarbonyl group, an optionally substituted alkyl group, an optionally substituted alkoxycarbonyl group or a nitro group;
provided that a partial structure (1-1): 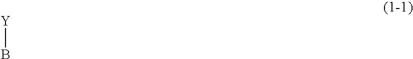
wherein the symbols have the same meanings as defined above, in the compound (1) is not a formula: 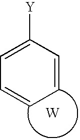
wherein a cyclic W is a heterocyclic group,
or a pharmaceutically acceptable derivative thereof.

5. The compound of claim 4 wherein Y is a methylene group optionally substituted by a group(s) selected independently from an alkyl group and an oxo group, or a single bond;
A¹ is a heterocyclic group or a single bond;
A² is an optionally substituted heterocyclic group, an optionally substituted alkoxy group, a halogen atom, an optionally substituted alkyl group, a cyano group, an amino group optionally substituted by 1 to 2 substituents, or a hydrogen atom;
B is a heterocyclic group optionally substituted by 1 to 5 groups selected independently from an oxo group, a cyano group, a carboxyl group, an optionally substituted alkoxycarbonyl group, an optionally substituted carbamoyl group, a hydroxyl group, an optionally substituted alkylsulfonyl group, an optionally substituted alkyl group, a halogen atom, an optionally substituted alkoxy group, an optionally substituted alkylsulfanyl group, an optionally substituted alkylsulfinyl group, an optionally substituted piperidyl group, and an amino group optionally substituted by 1 to 2 substituents;
R¹ is a hydrogen atom, an optionally substituted benzyl group or an optionally substituted phenylethyl group;
R² is an amino group optionally substituted by 1 to 2 substituents, a halogen atom, an optionally substituted alkoxy group, an optionally substituted carbamoyl group, an oxy group substituted by optionally substituted heterocyclic group, a hydroxy group, an optionally substituted heterocyclic group, an optionally substituted homocyclic group, an oxy group substituted by optionally substituted homocyclic group, a hydroxyalkyl group or a nitro group,
or a pharmaceutically acceptable derivative thereof.

6. The compound of claim 5 wherein Y is a methylene group optionally substituted by a group(s) selected independently from an alkyl group and an oxo group, or a single bond;
A¹ is a heterocyclic group or a single bond;
A² is an optionally substituted heterocyclic group, an optionally substituted alkoxy group, a halogen atom, an optionally substituted alkyl group, a cyano group, an amino group optionally substituted by 1 to 2 substituents, or a hydrogen atom;
B is a heterocyclic group optionally substituted by 1 to 5 groups selected independently from an optionally substituted alkyl group, a halogen atom, a hydroxy group, an optionally substituted cycloalkyl group, an optionally substituted cycloalkoxy group, an optionally substituted alkoxy group, an alkylsulfanyl group, an alkylsulfinyl group, an amino group optionally substituted by 1 to 2 substituents and an alkylsulfonyl group;
R¹ is 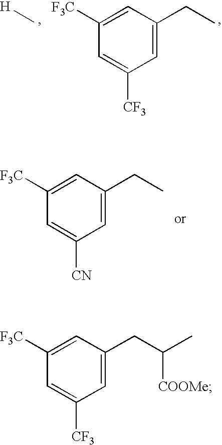
R² is an amino group optionally substituted by 1 to 2 substituents, a halogen atom, an optionally substituted alkoxy group, an optionally substituted carbamoyl group, an oxy group substituted by optionally substituted heterocyclic group, a hydroxy group, an optionally substituted heterocyclic group, an optionally substituted homocyclic group, an oxy group substituted by optionally substituted homocyclic group, an optionally substituted alkyl group, a hydrogen atom, an optionally substituted alkylcarbonyl group, an optionally substituted alkoxycarbonyl group, or a nitro group,
or a pharmaceutically acceptable derivative thereof.

7. The compound of claim 6 wherein the homocyclic group is a cycloalkyl group, a phenyl group or a naphthyl group;
the heterocyclic group is a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl or tetrahydroquinolyl group;
a substituent(s) for an optionally substituted alkyl group, an optionally substituted cycloalkyl group, an optionally substituted alkenyl group, an optionally substituted alkoxy group, an optionally substituted cycloalkoxy group, an optionally substituted alkoxycarbonyl group, an optionally substituted carbamoyl group, an optionally substituted carbamimidoyl group, an optionally substituted alkylsulfanyl group, an optionally substituted alkylsulfinyl group, an optionally substituted alkylsulfonyl group, an optionally substituted amino group, an optionally substituted sulfamoyl group, an optionally substituted alkanoyl group, an optionally substituted homocyclic group, an oxy group substituted by optionally substituted homocyclic group, a carbonyl group substituted by optionally substituted homocyclic group, an optionally substituted heterocyclic group, an oxy group substituted by optionally substituted heterocyclic group, a carbonyl group substituted by optionally substituted heterocyclic group, an optionally substituted phenyl group, an optionally substituted alkylsulfonyloxy group, an optionally substituted alkynyl group or an optionally substituted alkylene group is/are 1 to 5 groups selected independently from the following groups:
a halogen atom; a cyano group; a hydroxy group; a nitro group; a carboxyl group; an oxo group; a thioxo group; a sulfo group; a cycloalkyl group optionally substituted by hydroxy group, halogen atom, carboxyl group, alkoxycarbonyl group, mono- or di-alkylamino group, phenyl group or morpholinyl group; an alkoxycarbonyl group optionally substituted by hydroxy group, halogen atom, carboxyl group, alkoxycarbonyl group, mono- or di-alkylamino group, phenyl group or morpholinyl group; a carbamoyl group; a mono- or di-alkylcarbamoyl group optionally substituted by hydroxy group, halogen atom, carboxyl group, alkoxycarbonyl group, mono- or di-alkylamino group, phenyl group or morpholinyl group; an alkyl group optionally substituted by hydroxy group, halogen atom, carboxyl group, alkoxycarbonyl group, mono- or di-alkylamino group, phenyl group or morpholinyl group; an alkanoyl group optionally substituted by hydroxy group, halogen atom, carboxyl group, alkoxycarbonyl group, mono- or di-alkylamino group, phenyl group or morpholinyl group; an alkoxy group optionally substituted by hydroxy group, halogen atom, carboxyl group, alkoxycarbonyl group, mono- or di-alkylamino group, phenyl group or morpholinyl group; an alkanoyloxy group optionally substituted by hydroxy group, halogen atom, carboxyl group, alkoxycarbonyl group, mono- or di-alkylamino group, phenyl group or morpholinyl group; an alkylsulfanyl group optionally substituted by hydroxy group, halogen atom, carboxyl group, alkoxycarbonyl group, mono- or di-alkylamino group, phenyl group or morpholinyl group; an alkylsulfonyl group optionally substituted by hydroxy group, halogen atom, carboxyl group, alkoxycarbonyl group, mono- or di-alkylamino group, phenyl group or morpholinyl group; an alkylsulfinyl group optionally substituted by hydroxy group, halogen atom, carboxyl group, alkoxycarbonyl group, mono- or di-alkylamino group, phenyl group or morpholinyl group; a mono- or di-alkylsulfamoyl group optionally substituted by hydroxy group, halogen atom, carboxyl group, alkoxycarbonyl group, mono- or di-alkylamino group, phenyl group or morpholinyl group; an amino group; a mono- or di-alkylamino group optionally substituted by hydroxy group, halogen atom, carboxyl group, alkoxycarbonyl group, mono- or di-alkylamino group, phenyl group or morpholinyl group; a mono- or di-alkylsulfamoylamino group optionally substituted by hydroxy group, halogen atom, carboxyl group, alkoxycarbonyl group, mono- or di-alkylamino group, phenyl group or morpholinyl group; a mono- or di-alkylureido group optionally substituted by hydroxy group, halogen atom, carboxyl group, alkoxycarbonyl group, mono- or di-alkylamino group, phenyl group or morpholinyl group; a homocyclic group selected from a cycloalkyl group, a phenyl group and a naphthyl group; an oxy group substituted by the homocyclic group as defined above; a carbonyl group substituted by the homocyclic group as defined above; a heterocyclic group selected from a thienyl, furyl, pyrrolyl, pyrrolinyl, oxazolyl, thiazolyl, pyrazolyl, imidazolyl, imidazolinyl, isoxazolyl, isothiazolyl, oxadiazolyl, furazanyl, thiadiazolyl, triazolyl, triazinyl, triazolidinyl, tetrazolyl, pyridyl, imidazopyridyl, pyrimidinyl, thiomorpholinyl, morpholinyl, triazinyl, pyrrolidinyl, piperidyl, pyranyl, tetrahydropyranyl, thiopyranyl, oxazinyl, thiazinyl, piperazinyl, triazinyl, oxatriazinyl, pyridazinyl, pyrazinyl, benzofuranyl, benzothiazolyl, benzoxazolyl, tetrazolopyridazinyl, triazolopyridazinyl, benzimidazolyl, quinolyl, isoquinolyl, dihydroisoquinolyl, cinnolinyl, phthalazinyl, quinazolinyl, quinoxalinyl, indolizinyl, dihydroindolyl, indolyl, quinolizinyl, naphthyridinyl, purinyl, pteridinyl, dibenzofuranyl, carbazolyl, acridinyl, phenanthridinyl, chromanyl, benzoxazinyl, phenazinyl, phenothiazinyl, phenoxazinyl, hexahydroazepinyl, imidazolidinyl, oxazolidinyl, tetrahydrofuranyl, dioxolanyl, oxiranyl, dihydropyrimidinyl, oxazolinyl, dihydrooxazinyl, dihydropyrazolyl, imidazopyridyl, dihydropyrazinyl, tetrahydroquinolyl, benzothienyl, dihydrooxazolyl, oxathiadiazolyl, dihydrooxazolyl and tetrahydroquinolyl groups; an oxy group substituted by the heterocyclic group as defined above; a carbonyl group substituted by the heterocyclic group as defined above; and a group of the formulae: 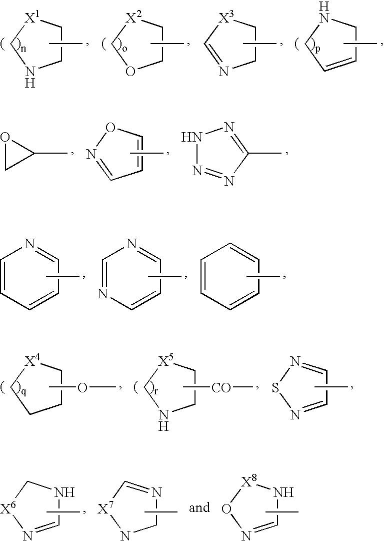
wherein X¹ and X³ are each independently CH₂, NH, O, S, SO or SO₂; X² and X⁵ are each independently CH₂, O, S, SO or SO₂; X⁴ is NH, O, S, SO or SO₂; X⁶ and X⁷ are each independently O or S; X⁸ is S or SO; and n, o, p, q and r are each independently an integer of 1 to 4, and further each of the above groups may optionally be substituted by 1 to 3 substituents selected from the following groups: halogen atom, carboxyl group, hydroxy group, cyano group, oxo group, thioxo group, alkyl group, hydroxyalkyl group, alkoxycarbonylalkyl group, carboxyalkyl group, morpholinylalkyl group, phenylalkyl group, alkanoyl group, hydroxyalkanoyl group, alkoxyalkanoyl group, alkoxy group, phenylalkoxy group, alkoxycarbonyl group, benzyloxycarbonyl group, mono- or di-alkylamino group, mono- or di-alkylcarbamoyl group, mono- or di-alkylsulfamoyl group, alkylsulfonyl group and tetrazolyl group,
or a pharmaceutically acceptable derivative thereof.

8. The compound of claim 7 wherein Y is a methylene group or a single bond;
A¹ is a pyrimidinyl group, a tetrazolyl group or a single bond;
A² is a mono- or di-alkylamino group optionally substituted by carboxyl group, a piperidyl group optionally substituted by carboxyl group, a morpholinyl group optionally substituted by carboxyl group, an alkoxy group optionally substituted by carboxyl group, a halogen atom, an alkyl group optionally substituted by carboxyl group, a hydrogen atom or a cyano group;
B is an isoquinolyl, tetrahydroquinolyl, quinolyl, pyrazolyl, pyridyl, pyrimidinyl, dihydroindolyl, imidazolyl or imidazopyridyl group; wherein said heterocyclic group may optionally be substituted by 1 to 5 substituents selected independently from the following groups: an oxo group; an alkoxy group optionally substituted by 1 to 3 halogen atoms; an alkyl group optionally substituted by 1 to 3 halogen atoms; a halogen atom; a hydroxy group; an alkylsulfanyl group; an alkylsulfinyl group; and an amino group optionally substituted by 1 to 2 substituents selected independently from an alkyl group or an alkyl group substituted by morpholinyl;
R¹ is a hydrogen atom, or a benzyl group substituted by 1 to 3 groups selected independently from an alkoxy group optionally substituted by cyano group and 1 to 3 halogen atoms and an alkyl group optionally substituted by 1 to 3 halogen atoms;
R² is (a) an amino group optionally substituted by 1 to 2 groups independently selected from an alkyl group, an alkoxyalkyl group, a cycloalkylalkyl group, an alkoxycarbonyl group, an alkylcarbonyl group, an alkylcarbamoyl group, a carboxyalkyl group, a cycloalkylalkyl group substituted by carboxyalkyl group, a hydroxyalkyl group, a carboxyalkoxycarbonyl group, a carboxydihydrooxazolyl group, a carboxyalkylcarbonyl group, a phenylalkyl group, an alkoxyalkoxycarbonyl group, an alkoxyalkylcarbonyl group, an alkyl group substituted by piperidyl group, a piperidylalkyl group substituted by carboxyalkyl group, and an alkyl group substituted by phenyl group that is optionally substituted by 1 to 2 alkyl groups (said alkyl group may optionally be substituted by 1 to 3 halogen atoms);
wherein said alkyl group or alkoxy group may further optionally be substituted by 1 to 5 groups selected independently from the following groups:
a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group optionally substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;
(b) a halogen atom;
(c) an alkoxy optionally substituted by a group selected from carboxyl group, cycloalkyl group and alkoxy group;
wherein said cycloalkyl group or alkoxy group may optionally be substituted by 1 to 5 groups selected independently from the following groups:
a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group optionally substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;
(d) a carbamoyl group optionally substituted by 1 to 2 substituents selected independently from alkyl group and carboxyalkyl group,
wherein said alkyl group may optionally be substituted by 1 to 5 groups selected independently from the following groups:
a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may be substituted by carboxyl, alkoxycarbonyl or hydroxy group), an alkoxy group (said alkoxy group may be substituted by carboxyl, formyl or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy or carboxyl group, a morpholinyl group, a pyrrolidinyl optionally substituted by alkoxycarbonyl or carboxyl group, a pyrrolidinyl optionally substituted by alkoxycarbonylalkyl or carboxyalkyl group, a pyrrolidinyl substituted by oxo group, a piperidyl optionally substituted by alkoxycarbonyl or carboxyl group, a piperidyl optionally substituted by alkoxycarbonylalkyl or carboxyalkyl group, a piperazinyl optionally substituted by alkyl group, a hexahydroazepinyl group, a pyrimidinyl group, a pyridyl group, a dioxolany group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;
(e) a hydroxy group;
(f) an oxy group substituted by a heterocyclic group selected from pyrimidinyl group and tetrahydropyranyl group;
(g) a heterocyclic group selected from a morpholinyl, piperidyl, piperazinyl, pyrazinyl, tetrazolyl, thienyl, furyl, dihydroisoquinolyl, pyridyl and pyrrolyl group, which are each optionally substituted by 1 to 2 substituents selected independently from pyrimidinyl group, halogen atom, alkyl group, cyano group, mono- or di-alkylamino group, alkoxy group, phenyl group, carboxyl group, carbamoyl group and carboxyalkyl group;
wherein said alkoxy group or alkyl group may optionally be substituted by 1 to 5 groups selected independently from the following groups:
a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;
(h) a phenyl group optionally substituted by 1 to 3 substituents selected independently from halogen atom, alkyl group and alkoxy group;
wherein said alkoxy group or alkyl group may further optionally be substituted by 1 to 5 groups selected independently from the following groups:
a halogen atom, a cyano group, a hydroxy group, a carboxyl group, an alkoxycarbonyl group, a tetrazolyl group, a carbamoyl group, a mono- or di-alkylcarbamoyl group (said mono- or di-alkylcarbamoyl group may optionally be substituted by carboxyl group, alkoxycarbonyl group or hydroxy group), an alkoxy group (said alkoxy group may optionally be substituted by carboxyl group, formyl group or hydroxy group), an alkanoyloxy group, an alkylsulfanyl group, an alkylsulfonyl group, an alkylsulfinyl group, an aminosulfonyl group, an amino group, a mono- or di-alkylamino group optionally substituted by carboxyl group or alkoxy group, a mono- or di-alkylsulfamoylamino group, a mono- or di-alkylureido group optionally substituted by morpholinyl group, a cycloalkyl group optionally substituted by carboxymethyl group, an oxiranyl group, a phenyl group optionally substituted by alkoxy group or carboxyl group, a morpholinyl group, a pyrrolidinyl group optionally substituted by alkoxycarbonyl group or carboxyl group, a pyrrolidinyl group optionally substituted by alkoxycarbonylalkyl group or carboxyalkyl group, a pyrrolidinyl group substituted by oxo group, a piperidyl group optionally substituted by alkoxycarbonyl group or carboxyl group, a piperidyl group optionally substituted by alkoxycarbonylalkyl group or carboxyalkyl group, a piperazinyl group optionally substituted by alkyl group, a hexahydroazepinyl group, a pyrimidinyl group, a pyridyl group, a dioxolanyl group optionally substituted by alkyl group, an oxadiazolyl group optionally substituted by oxo group, an oxathiadiazolyl group optionally substituted by oxo group, a pyrrolidinylcarbonyl group optionally substituted by carboxyl group, a piperidyloxy group optionally substituted by alkyl group and a morpholinylcarbonyl group;
(i) an oxy group substituted by cycloalkyl group;
(j) a hydroxyalkyl group; or
(k) a nitro group,
or a pharmaceutically acceptable derivative thereof.

9. The compound of claim 8 wherein Y is a methylene group or a single bond;
A¹ is a pyrimidinyl group, a tetrazolyl group or a single bond;
A² is a mono- or di-alkylamino group optionally substituted by carboxyl group, a piperidyl group optionally substituted by carboxyl group, a morpholinyl group optionally substituted by carboxyl group, alkoxy group optionally substituted by carboxyl group, a halogen atom, an alkyl group optionally substituted by carboxyl group, a hydrogen atom or a cyano group;
B is an isoquinolyl, tetrahydroquinolyl, quinolyl, pyrazolyl, pyridyl, pyrimidinyl, dihydroindolyl, imidazolyl or imidazopyridyl group; wherein said heterocyclic group may optionally be substituted by 1 to 5 substituents selected independently from the following groups: an oxo group; an alkoxy group optionally substituted by 1 to 3 halogen atoms; an alkyl group optionally substituted by 1 to 3 halogen atoms; a halogen atom; an alkylsulfanyl group; an alkylsulfinyl group; and an amino group optionally substituted by 1 to 2 substituents selected independently from an alkyl group or an alkyl group substituted by morpholinyl group;
R¹ is 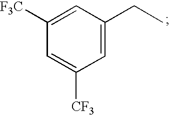
R² is (a) an amino group optionally substituted by 1 to 2 groups selected independently from an alkyl group, an alkoxyalkyl group, a cycloalkylalkyl group, an alkoxycarbonyl group, an alkylcarbonyl group, an alkylcarbamoyl group, a carboxyalkyl group, a cycloalkylalkyl group substituted by carboxyalkyl group, a hydroxyalkyl group, a carboxyalkoxycarbonyl group, a carboxydihydrooxazolyl group, a carboxyalkylcarbonyl group, a phenylalkyl group, an alkoxyalkoxycarbonyl group, an alkoxyalkylcarbonyl group, an alkyl group substituted by piperidyl group, a piperidylalkyl group substituted by carboxyalkyl group and an alkyl group substituted by phenyl group that is optionally substituted by 1 to 2 alkyl groups (said alkyl group may optionally be substituted by 1 to 3 halogen atoms);
(b) a halogen atom;
(c) an alkoxy group optionally substituted by a group selected from a carboxyl group, a cycloalkyl group and an alkoxy group;
(d) a carbamoyl group optionally substituted by 1 to 2 substituents selected independently from alkyl group and carboxyalkyl group;
(e) a hydroxy group;
(f) an oxy group substituted by a heterocyclic group selected from a pyrimidinyl group and a tetrahydropyranyl group;
(g) a heterocyclic group selected from a morpholinyl, piperidyl, pyrazinyl, dihydroisoquinolyl, pyridyl, pyrimidinyl, piperazinyl, tetrazolyl or pyrrolyl group, which are each optionally substituted by 1 to 3 substituents selected independently from alkyl group, halogen atom, phenyl group, alkoxy group, carboxyl group and carboxyalkyl group;
(h) a phenyl group optionally substituted by 1 to 3 substituents selected independently from halogen atom, alkyl group and alkoxy group;
(i) an oxy group substituted by cycloalkyl group;
(j) a hydroxyalkyl group;
(k) a nitro group;
or a pharmaceutically acceptable derivative thereof.

10. The compound of claim 9 wherein Y is a methylene group;
A¹ is a pyrimidinyl group;
A² is an alkoxy group optionally substituted by carboxyl group, a piperidyl group optionally substituted by carboxyl group, an amino group optionally substituted by 1 to 2 groups selected independently from carboxyalkyl group and alkyl group, or an alkyl group substituted by carboxyl group;
B is a pyrimidinyl, pyridyl, quinolyl, dihydroindolyl, pyrazolyl, isoquinolyl, imidazolyl or imidazopyridyl group; wherein said heterocyclic group may optionally be substituted by 1 to 5 substituents selected independently from the following groups: an oxo group; an alkoxy group optionally substituted by 1 to 3 halogen atoms; an alkyl group optionally substituted by 1 to 3 halogen atoms; a halogen atom; an alkylsulfanyl group; an alkylsulfinyl group; and an amino group optionally substituted by 1 to 2 substituents selected independently from an alkyl group or an alkyl group substituted by morpholinyl;
R¹ is 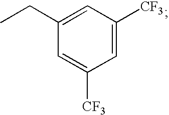
R² is (a) an amino group optionally substituted by 1 to 2 groups selected independently from a carboxyalkyl group, an alkoxyalkyl group, a hydroxyalkyl group, mono- or di-alkylaminoalkyl group, an alkyl group, a cycloalkylalkyl group and an alkoxycarbonyl group;
(b) an alkoxy group;
(c) a phenyl group optionally substituted by 1 to 2 substituents selected independently from an alkyl group and an alkoxy group;
(d) a pyrimidinyl group optionally substituted by 1 to 2 substituents selected from an alkoxy group and an alkyl group;
(e) a pyridyl group optionally substituted by 1 to 2 substituents selected from an alkoxy group and an alkyl group,
or a pharmaceutically acceptable derivative thereof.

11. A compound described in any one of examples No. 1, 4, 5, 14, 21 and 23, or a pharmaceutically acceptable derivative thereof.

12. A compound of the formula (I-A): 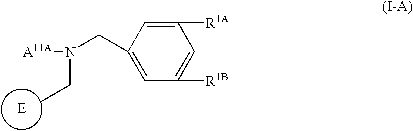
wherein
A^{11A} is an optionally substituted pyrimidin-2-yl group;
R^{1A} and R^{1B} are independently a cyano group or an alkyl group optionally substituted by 1 to 3 halogen atoms;
Ring E is an optionally substituted heterocyclic group which contain 1 to 2 nitrogen atoms,
or a pharmaceutically acceptable derivative thereof.

13. A compound of the formula (I-B): 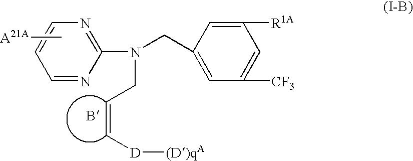
wherein
A^{21A} is an optionally substituted carbamoyl group, an optionally substituted homocyclic group, an optionally substituted alkylsulfanyl group, an optionally substituted alkylsulfinyl group, an optionally substituted alkylsulfonyl group, an optionally substituted alkyl group, a nitro group, a hydroxy group, a cyano group, an optionally substituted alkenyl group, an optionally substituted heterocyclic group, an optionally substituted alkoxy group, a halogen atom, an amino group optionally substituted by 1 to 2 substituents, a carboxyl group or a hydrogen atom; R^{1A} is a cyano group or an alkyl group optionally substituted by 1 to 3 halogen atoms;
B′ is optionally substituted by 1 to 3 groups selected independently from an oxo group, a cyano group, a halogen atom, a hydroxy group, an optionally substituted cycloalkyl group, an optionally substituted cycloalkoxy group, an optionally substituted heterocyclic group, an optionally substituted alkylsulfanyl group, an optionally substituted alkylsulfinyl group, an optionally substituted alkylsulfonyl group, an amino group optionally substituted by 1 to 2 substituents, an optionally substituted alkyl group and an optionally substituted alkoxy group;
D is a phenyl group, a tetrazol-5-yl group, a pyrimidin-2-yl group, a pyrimidin-4-yl group, a pyrimidin-5-yl group, a pyrimidin-6-yl group, a pyridin-2-yl group, a pyridin-3-yl group, a pyridin-4-yl group, a pyridin-5-yl group, a pyridin-6-yl group, or a hydrogen atom;
D′ is a group selected independently from a halogen atom, an alkoxyalkyl group, an alkyl group substituted by 1 to 5 halogen atoms, an alkoxy group substituted by 1 to 5 halogen atoms, an alkenyl group, a carbamoyl group, a cycloalkyl group, a mono- or di-alkylaminoalkyl group, a mono- or di-alkylaminoalkoxy group, a carboxyl group, a hydroxy group, a cyano group, an oxo group, an alkyl group, a hydroxyalkyl group, an alkoxycarbonylalkyl group, a carboxyalkyl group, a morpholinylalkyl group, a phenylalkyl group, an alkanoyl group, a hydroxyalkanoyl group, an alkoxyalkanoyl group, an alkoxy group, a phenylalkoxy group, an alkoxycarbonyl group, a benzyloxycarbonyl group, a mono- or di-alkylamino group, a mono- or di-alkylcarbamoyl group, a mono- or di-alkylsulfamoyl group, an alkylsulfonyl group, a tetrazolyl group, a benzyloxyalkyl group, a cycloalkylalkyl group, a benzyloxy group, an alkoxyalkoxy group, a carboxyalkoxy group, a carboxyalkenyl group, an alkylcarbonylamino group, a carboxyalkoxyalkyl group, a morpholinyl group or a pyridylalkoxy group;
q^{A} is an integer of 0 to 3; 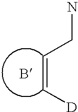
is a group selected from the following groups: 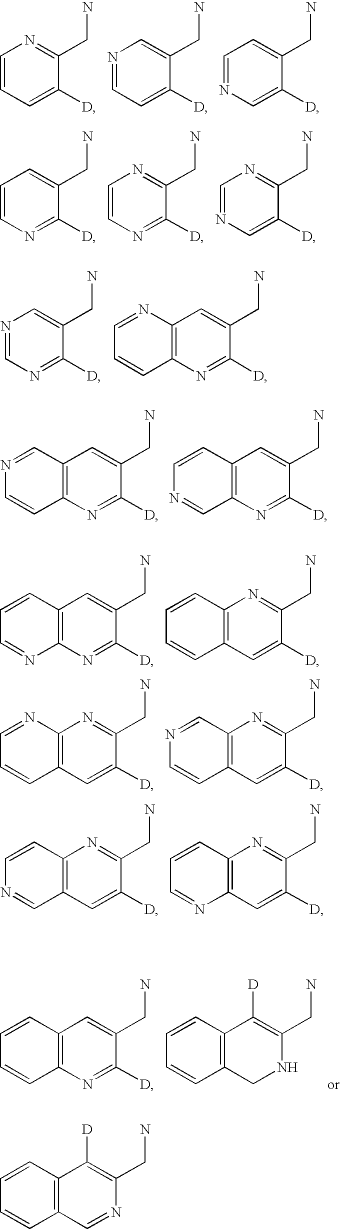
, or a pharmaceutically acceptable derivative thereof.

14. The compound of claim 13 wherein
A^{21A} is selected from the following group:
(a) a heterocyclic group selected from a piperidyl group and a morpholinyl group, respectively optionally substituted by a substituent(s) selected from a carboxyl group, an alkoxycarbonyl group, a carboxyalkyl group or an alkyl group;
(b) an alkoxy group optionally substituted by a group selected from a carboxyl group, an alkoxycarbonyl group, a halogen atom, an alkylsulfinyl group, a mono- or di-alkylamino group, a cyano group, a tetrazolyl group, an alkylsulfonyl group, an alkylsulfanyl group, a hydroxy group or an alkoxy group;
(c) a halogen atom;
(d) an amino group optionally substituted by 1 to 2 substituents independently selected from a carboxyalkyl group, an alkoxycarbonylalkyl group, an alkylsulfonylalkyl group, an alkylsulfinylalkyl group, a hydroxyalkyl group, an alkyl group, an alkoxyalkyl group or an aminoalkyl group optionally substituted by 1 to 2 alkyl groups;
(e) a hydrogen atom;
(f) an alkyl group optionally substituted by a group selected from a carboxyl group, an alkoxycarbonyl group, a halogen atom, an alkylsulfinyl group, a mono- or di-alkylamino group, a cyano group, a tetrazolyl group, an alkylsulfonyl group, an alkylsulfanyl group, a hydroxy group or an alkoxy group;
(g) a carboxyl group;
(h) a carbamoyl group optionally substituted by a carboxyalkyl group;
(i) an alkenyl group substituted by a group selected from a carboxyl group, an alkoxycarbonyl group, an alkylsulfinyl group, a cyano group, a tetrazolyl group, an alkylsulfonyl group, an alkylsulfanyl group, a hydroxy group or an alkoxy group;
(j) a morpholinyl group;
(k) a piperidinyl group optionally substituted by a carboxyl group or a carboxyalkyl group;
B′ is optionally substituted by 1 to 3 groups selected independently from an oxo group, a halogen atom, an alkyl group optionally substituted by 1 to 3 halogen atoms, an alkoxy group optionally substituted by 1 to 3 halogen atoms, a cyano group, an alkylsulfanyl group optionally substituted by 1 to 3 halogen atoms, an alkylsulfinyl group optionally substituted by 1 to 3 halogen atoms, an amino group, an alkoxyalkyl group, a benzyloxy group, an alkoxyalkoxy group, a mono or di-alkylaminoalkoxy group, a mono or di-alkylaminoalkyl group, a cycloalkyl group, a cycloalkoxy group and an alkylsulfonyl group optionally substituted by 1 to 3 halogen atoms,
or a pharmaceutically acceptable derivative thereof.

15. The compound of claim 14 wherein
A^{21A} is a morpholinyl group, a carboxyalkoxy group, an alkylsulfonylalkoxy group, a carboxyalkenyl group, a carboxyalkyl group, an alkoxycarbonylalkoxy group, an alkoxycarbonylalkyl group or a carboxypiperidinyl group;
B′ is optionally substituted by 1 to 3 groups selected independently from an alkyl group optionally substituted by 1 to 3 halogen atoms, a halogen atom, an alkoxy group optionally substituted by 1 to 3 halogen atoms and an oxo group;
D′ is a group selected independently from a benzyloxyalkyl group, a halogen atom, a cycloalkylalkyl group, an alkyl group optionally substituted by 1 to 3 halogen atoms or an alkoxy group optionally substituted by 1 to 3 halogen atoms; 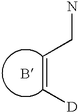
is a group selected from the following groups: 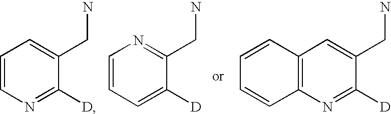
, or a pharmaceutically acceptable derivative thereof.

16. The compound of claim 15, wherein
A^{21A} is a carboxyalkoxy group or a carboxypiperidinyl group;
D is a phenyl group;
D′ is a group selected independently from a halogen atom, an alkoxy group or an alkyl group,
or a pharmaceutically acceptable derivative thereof.

17. The compound of claim 15, wherein
A^{21A} is a carboxyalkoxy group;
B′ is optionally substituted by 1 to 3 groups selected independently from an alkyl group optionally substituted by 1 to 3 halogen atoms and an alkoxy group optionally substituted by 1 to 3 halogen atoms;
D is a phenyl group;
D′ is a group selected independently from a halogen atom, an alkoxy group or an alkyl group; 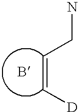
is 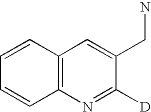
, or a pharmaceutically acceptable derivative thereof.

18. A compound described in any one of examples No. 52, 54, 55, 57, 58, 60, 63, 65, or a pharmaceutically acceptable derivative thereof.

19. A compound described in any one of examples No. 77 to 84, or a pharmaceutically acceptable derivative thereof.

20. A pharmaceutical composition, which comprises as an active ingredient a compound according to claim 1, or a pharmaceutically acceptable derivative thereof.

21. A method for prophylaxis or treatment of arteriosclerosis such as atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular diseases, angina, ischemia, cardiac ischemia, stroke, myocardial infarction, reperfusion injury, restenosis after angioplasty, hypertension, cerebral infarction, cerebral stroke, diabetes, vascular complication of diabetes, thrombotic diseases, obesity, endotoxemia, metabolic syndrome, cerebrovascular disease, coronary artery disease, ventricular dysfunction, cardiac arrhythmia, pulmonary vascular disease, reno-vascular disease, renal disease, splanchnic vascular disease, vascular hemostatic disease, fatty liver disease, steatohepatitis, inflammatory disease, autoimmune disorders and other systemic disease indications, immune function modulation, pulmonary disease, anti-oxidant disease, sexual dysfunction, cognitive dysfunction, schistosomiasis, cancer, regression of xanthoma or Alzheimer's disease, which comprises administering an effective amount of a compound according to claim 1, or a pharmaceutically acceptable derivative thereof, to a patient in need thereof.

22. Use of a compound according to claim 1, or a pharmaceutically acceptable derivative thereof, for the manufacture of a medicament in treatment of patients suffering from arteriosclerosis such as atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular diseases, angina, ischemia, cardiac ischemia, stroke, myocardial infarction, reperfusion injury, restenosis after angioplasty, hypertension, cerebral infarction, cerebral stroke, diabetes, vascular complication of diabetes, thrombotic diseases, obesity, endotoxemia, metabolic syndrome, cerebrovascular disease, coronary artery disease, ventricular dysfunction, cardiac arrhythmia, pulmonary vascular disease, reno-vascular disease, renal disease, splanchnic vascular disease, vascular hemostatic disease, fatty liver disease, steatohepatitis, inflammatory disease, autoimmune disorders and other systemic disease indications, immune function modulation, pulmonary disease, anti-oxidant disease, sexual dysfunction, cognitive dysfunction, schistosomiasis, cancer, regression of xanthoma or Alzheimer's disease.
